(19) United States
(12) Patent Application Publication
(10) Pub. No.: US 2018/0125961 A1
(43) Pub. Date: May 10, 2018

(54) NEISSERIA MENINGITIDIS TRYPSIN-LIKE SERINE PROTEASE POLYPEPTIDES AND COMPOSITIONS THEREOF

(71) Applicant: SANOFI PASTEUR, Lyon (FR)

(72) Inventors: Nadège ARNAUD-BARBE, Saint Sorlin (FR); Geneviève RENAULD-MONGENIE, Chaponost (FR); Bachra ROKBI, Lyon (FR)

(21) Appl. No.: 15/534,443

(22) PCT Filed: Dec. 8, 2015
(86) PCT No.: PCT/EP2015/079035

§ 371 Date: Jun. 8, 2017

(30) Foreign Application Priority Data
Dec. 9, 2014 (EP) 14306977.1

(51) Int. Cl. A61K 39/095; C12N 9/52

(52) U.S. Cl. CPC A61K 39/095; C07K 2319/40; C12N 9/52; C12Y 304/21072

(57) ABSTRACT

The present invention relates to novel polypeptides derived from Neisseria meningitidis proteins, in particular auto-transporters of the trypsin-like serine protease subclass, such as IgA1P, App and AusI, and their use in immunogenic compositions i.a., vaccine compositions for the prevention and/or treatment of meningococcal infections. In particular, it provides fragments of IgA1P, App and AusI and polypeptides comprising or consisting of these fragments and fusions thereof, which may be used in immunogenic compositions, for example vaccine compositions.

### FIELD OF THE INVENTION

The present invention relates to novel polypeptides derived from Neisseria meningitidis proteins, in particular auto-transporters of the trypsin-like serine protease subclass, such as IgA1P, App and AusI, and their use in immunogenic compositions, and in particular in vaccine compositions for the prevention and/or treatment of meningococcal infections. In particular, it provides fragments of IgA1P, App and AusI and polypeptides comprising or consisting of these fragments and fusions thereof, which may be used in immunogenic compositions, in particular in vaccine compositions.

### BACKGROUND

Neisseria meningitidis is one of the most important causes of bacterial meningitis and septicemia worldwide in both endemic and epidemic forms. The bacteria are classified into serogroups based on the structure of their capsular polysaccharides. Thirteen different serogroups have been identified but only five (A, B, C, W135 and Y) are responsible for the majority of infections, although epidemic meningitis due to meningococcal serogroup X is emerging in the Meningitis Belt of Africa. Two effective quadrivalent polysaccharide-protein conjugate vaccines have been developed and licensed against serogroups A, C, W135 and Y. In contrast to the other capsular polysaccharides, the group B polysaccharide is not an appropriate vaccinal antigen because of structural similarities with polysialic acid chains present in human cells. These properties of the serogroup B polysaccharide have impeded the development of a polysaccharide-based vaccine against group B Neisseria meningitidis (MenB) and led to the development of alternative vaccines.

During in vitro culture conditions and in vivo infection N. meningitidis releases outer membrane blebs which contain lipooligosaccharide (LOS) and outer membrane proteins (OMPs). These blebs are known as outer membrane vesicles (OMVs). Meningococcal OMV vaccines have been developed and shown to be successful in controlling outbreaks of MenB disease when using OMVs produced from the outbreak strain. Several approaches have been carried out to increase the breadth of coverage of OMV vaccines. Despite these developments and the suggestion that a vaccine including six PorA and five FetA variants would potentially provide protection against the majority of global circulating pathogenic strains, the search for a vaccine candidate that is highly conserved and expressed by all disease causing meningococci has continued.

Recently, a vaccine against N. meningitidis has been licensed and commercialized under the trade name Bexsero™. This vaccine contains three recombinant N. meningitidis serogroup B proteins, namely NHBA (Neisseria Heparin Binding Antigen) fusion protein, NadA protein and fHbp fusion protein, together with outer membranes vesicles (OMVs) from N. meningitidis serogroup B.

Reference to N. meningitidis herein may be understood to refer to any serogroup, or may be understood to refer specifically to the B serogroup.

Meninge outer membrane proteins (OMPs) are now considered as the most promising vaccine candidates for broadly protecting against all serogroups. A particular class of OMPs is constituted by auto-transporters.

Auto-transporters are virulence factors produced by Gram-negative bacteria. Auto-transporters are modular proteins initially expressed as a precursor consisting of an N-terminal signal sequence and a C-terminal translocator domain separated by a N-terminal passenger domain that is secreted into the extra cellular medium. The signal sequence directs the auto-transporter to the secretion machinery for transport across the internal membrane. The translocator domain mediates the transport of the passenger domain across the outer membrane. The term auto-transporter was coined because of the apparent absence of dedicated secretion machinery.

On the basis of their N- and C-terminus domains, auto-transporters can be divided into several categories. The classical auto-transporters, as typified by the IgA1 protease (IgA1P), contain a catalytic site in the N-terminal half of the passenger domain which often, although not always, is involved in the autocatalytic release of the passenger domain from the cell surface. The catalytic site is constituted by an amino acid triad comprising a serine residue. Accordingly, these auto-transporters are classified as serine proteases.

The very first N. meningitidis auto-transporter that was described was indeed the IgA1 protease (IgA1P). The amino acid sequence precursor of this protein was first described in Lomholt et al., Mol. Microb. (1995) 15 (3): 495. Since then, the N. meningitidis IgA1 protease has been extensively studied and characterized (Vitovski & Sayers, Infect. Immun. (2007) 75 (6): 2875; Ulsen & Tommassen, FEMS Microbiol. Rev. (2006) 30 (2): 292). The IgA1 protease was proposed for vaccine use in the early nineties (WO 90/11367).

After becoming publicly available, the genomes of N. meningitidis strains MC58 (serogroup B) (Tettelin et al., Science (March 2000) 287: 1809), Z2491 (serogroup A) and FAM18 (serogroup C) have been systematically searched for the presence of genes encoding auto-transporters. BLAST searches using known auto-transporters as search leads resulted in the identification of eight genes putatively encoding proteins with auto-transporter characteristics, four of which encode serine proteases:
iga encoding IgA1 protease (IgA1P): NMB 0700, NMA 0905 and NMC 0651 respectively in the MC58 (serogroup B), Z2491 (serogroup A) and FAM18 (serogroup C) strain genomes;
app encoding App (adhesion and penetration protein): NMB 1985, NMA 0457 and NMC 1969 respectively in the MC58, Z2491 and FAM18 strain genomes;
ausI (mspA) encoding AusI (MspA): NMB 1998 in the MC58 genome; and
nalP (aspA) encoding NalP (AspA): NMB 1969, NMA 0478 and NMC 1943 respectively in the MC58, Z2491 and FAM18 strain genomes.

While NalP is known to be a subtilisin-like serine protease, IgA1P, App and AusI are classified as being chymotrypsin-like serine proteases (herein after called trypsin-like serine proteases). IgA1P, App and AusI are different proteins displaying similar tridimensional structure, as they are all auto-transporters, and some sequence homology at least in the passenger domain, including the catalytic triad.

The translocator domain of trypsin-like serine proteases is essential for the transport of the passenger domain across the membrane. It is constituted of several hydrophobic beta-sheets integrated into the outer-membrane that form a channel through which the passenger domain is secreted and is accordingly designated under the term “beta-domain”. Typically, the IgA1P beta-domain contains twelve beta-sheets (Gripstra et al., Res. in Microbiol. (2013) 164: 562).

As described in Pohlner et al., Nature (1987) 325: 458, the ˜160 kDa passenger domain of IgA1P essentially consists of two sub-domains: (i) the N-terminal sub-domain containing the protease activity (protease sub-domain) and (ii) a ˜40 kDa alpha-peptide domain which are connected together via a small gamma-peptide. The two sub-domains can be released separately or as a single polypeptide.

For ease of description, the protease sub-domain together with the gamma peptide is referred hereinafter as a single entity under the term “protease domain”. The protease sub-domain extends from the N-terminal end of the mature IgA1P polypeptide, to the PAP|SP auto-cleavage site.

As a matter of example, the amino acid sequence of the IgA1P precursor of MC58 (NMB0700) is shown in SEQ ID NO: 1. Further details are to be found in Table 1A below.

App and AusI were studied more recently [van Ulsen et al., FEMS Immunol Med. Microb. (2001) 32: 53; Serruto et al., Mol. Microb. (2003) 48 (2): 323; van Ulsen et al., Microbes & Infection (2006) 8: 2088; Turner et al., Infect. Immun. (2006) 74 (5): 2957; Ulsen & Tommassen (supra); Henderson et al., Microbiol. Mol. Biol. Rev. (2004) 68 (4): 692]. App and AusI are both trypsin-like serine proteases, with FINTL as putative auto-cleavage site. Although the boundaries of their domains and sub-domains are less characterized than those of IgA1P, there is no doubt that they share the same domain organization as is apparent from Tables 1B and 10. Genome analysis shows that App is quite conserved in N. meningitidis, with sequence identities compared with MC58 App being from 88 to 98%.

TABLE 1A
Domain structure of MC58 IgA1P (NMB0700)

| | | | Passenger domain | | | |
|---|---|---|---|---|---|---|
| Serine | | | Protease domain | | Alpha- | |
| protease | Catalytic | Signal | Protease | Gamma- | peptide | |
| motif | triad | sequence | sub-domain | peptide | domain | Beta-domain |
| 265-270 | 101H | 1-27 | 28-975 | 976-1007 | 1008-1505 | 1506-1815 |
| GDSGSP | 150D | | PAP | SP | | |
| | 267S | | | | | |

TABLE 1B
Domain structure of MC58 App (NMB1985)

| | | | Passenger domain | | | |
|---|---|---|---|---|---|---|
| Serine | | | Protease domain | | Alpha- | Beta-domain |
| protease | Catalytic | Signal | Protease | | peptide | (prediction |
| motif | triad | sequence | sub-domain | | domain | Uniprot) |
| 265-270 | 115H | 1-41 | 42-956 | 957-1056 | 1057-1178 | 1205-1457 |
| GDSGSP | 158D | | 956 F | NTL | 1057-1204* | |
| | 267S | | | | | |

*based on pertactin

TABLE 1C
Domain structure of MC58 Ausl (NMB1998)

| | | | Passenger domain | | | |
|---|---|---|---|---|---|---|
| Serine | | | Protease domain | | Alpha- | Beta-domain |
| protease | Catalytic | Signal | Protease | | peptide | (prediction |
| motif | triad | sequence | sub-domain | | domain | Uniprot) |
| 239-244 | 100H | 1-26 | 27-870 | 871-968 | 969-1177 | 1178-1431 |
| GDSGSP | 135D | | 870 F | NTL | 969-1131* | |
| | 241S | | | | | |

*based on pertactin

The boundaries of the IgA1P, App and AusI domains may vary slightly, as it is not always possible to precisely define a domain to the exact amino acid. For example, the domains may be defined slightly different depending on the methods/techniques used by different scientists to identify them and on the strain origin of the sequences. Thus, the domains indicated in Table 1A to 10 may be defined according to the locations given herein and/or in the Figures, or according to said locations +/−1, 2, 3, 4, 5, 6, 7, 8, 9, 10 or more amino acids, i.e. 1, 2, 3, 4, 5, 6, 7, 8, 9, 10 or more amino acids N-terminal or C-terminal of said locations. A ‘domain’ of a trypsin-like serine protease auto-transporter protein as referred to herein may be said domain as defined Table 1A, 1B or 10, or may be said domain +/−1, 2, 3, 4, 5, 6, 7, 8, 9, 10 or more amino acids at the N-terminus and/or the C-terminus of said locations.

### DESCRIPTION OF THE INVENTION

Trypsin-like serine proteases are proteins with low solubility due to the presence of the beta-domain and as a consequence of this, recombinant expression and purification of full-length trypsin-like serine proteases may be difficult to achieve.

Surprisingly, it has now been found that fragments of these proteins lacking all or at least a portion of the beta-domain may be conveniently expressed and/or purified (improved expression profiles, improved solubility); and that these fragments may be useful immunogens. In particular, those fragments may have improved vaccine potential when the passenger domain cannot be auto-cleaved.

Fusion fragments comprising the protease domain or sub-domain from one of the trypsin-like serine protease auto-transporters and at least the alpha-peptide domain from another of the trypsin-like serine protease auto-transporters were also found to be useful immunogens.

Thus, the invention provides a polypeptide, preferably an isolated polypeptide, selected from polypeptides comprising or consisting of:

(I):

(A) a fragment of a full-length mature trypsin-like serine protease auto-transporter of N. meningitidis, said fragment consisting of:
(i) a protease domain of a trypsin-like serine protease auto-transporter of N. meningitidis; or
(ii) a protease domain and all or part of an α-peptide domain of a trypsin-like serine protease auto-transporter of N. meningitidis; or
(iii) a protease domain, an α-peptide domain and a part of a β-domain, in particular a part of the β-domain comprising at least one and no more than eleven β-sheets of a trypsin-like serine protease auto-transporter of N. meningitidis;
   or

(B) a mutant of said fragment (A) which lacks or has reduced trypsin-like serine protease activity and/or does not contain any cleavage site able/susceptible to be cleaved by a trypsin-like serine protease;

wherein said polypeptide under (A) or (B) does not comprise the said full-length mature trypsin-like serine protease auto-transporter of N. meningitidis;
or

(II):

a first fragment fused to a second fragment:

(1) said first fragment consisting of:
a protease domain or a protease sub-domain of a first trypsin-like serine protease auto-transporter of N. meningitidis, or
a mutant of a protease domain or a protease sub-domain of a first trypsin-like serine protease auto-transporter of N. meningitidis which lacks or has reduced trypsin-like serine protease activity and/or does not contain any cleavage site able/susceptible to be cleaved by a trypsin-like serine protease,

(2) said second fragment consisting of:
an α-peptide domain, and optionally a part of a β-domain, of a second trypsin-like serine protease auto-transporter of N. meningitidis;

wherein the first and second trypsin-like serine protease auto-transporters are different; and

wherein the C-terminus of the first fragment is fused to the N-terminus of the second fragment,

wherein said polypeptide does not comprise the said full-length mature trypsin-like serine protease auto-transporter of N. meningitidis.

According to one embodiment, a trypsin-like serine protease auto-transporter of N. meningitidis suitable for the invention may be IgA1P, App or AusI.

Polypeptides described under (I) (A) and (B) herein above are collectively referred to herein after as “Fragment Polypeptides”. Polypeptides described under (II) herein above are collectively referred to herein after as “Fusion Polypeptide”.

By “full-length mature trypsin-like serine protease auto-transporter” is meant the trypsin-like serine protease auto-transporter lacking the signal peptide.

Accordingly, by “full-length mature trypsin-like serine protease auto-transporter” is meant the full-length mature trypsin-like serine protease auto-transporter comprising (having) (i) a naturally-occurring full-length mature amino acid sequence or (ii) a naturally-occurring full-length mature amino acid sequence lacking at most the first 50, 40, 30, 20, 10 or 5 N-terminus amino acids and/or at most the last 5 C-terminus amino acids or (iii) a naturally-occurring full-length mature amino acid sequence fused to the 1, 2 or 3 amino acids of the C-terminus of the signal sequence.

As used herein, the term “fragment” of a reference sequence or sequences refers to a chain of contiguous nucleotides or amino acids that is shorter than the reference sequence or sequences.

An ‘isolated’ peptide, protein or nucleic acid may be isolated substantially away from one or more elements with which it is associated in nature, such as other naturally occurring peptide, protein or nucleic acid sequences.

Although the polypeptides of the present invention are more particularly exemplified herein by reference to the amino acid sequence of MC58 IgA1P, App and AusI proteins (that naturally-occur in N. meningitidis strain MC58), polypeptides of any naturally-occurring/allelic variant of N. meningitidis strain MC58 are also encompassed within the scope invention as well as any variant that may result from genetic engineering.

By extension, the term variant is therefore applied to amino acid sequences, proteins or fragments thereof other than MC58 sequences, proteins or fragments thereof. Variations in amino acid sequence may be introduced by substitution, deletion or insertion of one or more codons into the nucleic acid sequence encoding the protein that results in a change in the amino acid sequence of the protein without substantially affecting the tri-dimensional structure and/or the biological and/or immunogenic properties. Typically, the variation may result for an amino acid substitution that may be conservative or non-conservative, preferably conservative. A conservative substitution is an amino acid substitution in which an amino acid is substituted for another amino acid with similar structural and/or chemical properties.

In what follows, variants are described by reference to the amino acid sequence of reference (SEQ ID NOs: 1-6). Such a description by reference is based on the prerequisite of optimal sequence alignment in order to determine i.a., the amino acid in the variant sequence that corresponds to the amino acid defined as being in a specific position in the amino acid of reference.

In what follows, variants and/or mutants are also described by percent identity with a sequence of reference. Percent identity between two amino acid sequences or two nucleotide sequences is determined with standard alignment algorithms as those described below.

Depending on the need, sequence alignment can alternatively be achieved and percent identity can also be determined by standard local alignment algorithms such as the Smith-Waterman algorithm (Smith et al., J. Mol. Biol. (1981) 147: 195) (available on the EBI web site) or Basic Local Alignment Tools (BLASTs, including BLASTP for amino acid sequence alignment and BLASTN for nucleotide sequence alignment; described in Altschul et al., (1990) J. Mol. Biol., 215: 403) available on the National Center for the Biotechnology Information (NCBI) web site at http://www.ncbi.nlm.nih.gov/BLAST and may be used using the default parameters. As a matter of example, default parameters for BLASTs, in July 2014 are:
For BLASTP: Expect value E: 10; Word size: 3; Matrix: BLOSUM62; Cost gap: Existence 11, Extension 1; and
For BLASTN: algorithm by default: Megablast; Expect value E: 10; Word size: 28; Match scores: 1; Mismatch score: −2; Gap costs: linear (determined by the match/mismatch score.

In the context of the invention, variant and mutant amino acid sequences include amino acid sequences that have at least about 80% sequence identity with an amino acid sequence defined herein. Preferably, a variant amino acid sequence will have at least about 80%, 81%, 82%, 83%, 84%, 85%, 86%, 87%, 88%, 89%, 90%, 91%, 92%, 93%, 94%, 95%, 96%, 97%, 98%, 99% sequence identity to a polypeptide sequence as defined herein. Amino acid sequence identity is defined as the percentage of amino acid residues in the variant sequence that are identical with the amino acid residues in the reference sequence, after aligning the sequences and if necessary, introducing gaps, to achieve the maximum percent sequence identity, and not considering any conservative substitution as part of the sequence identity. Standard alignment algorithms cited above are useful in this regard.

Variant and mutant nucleic acid sequences may include nucleic acid sequences that have at least about 80% sequence identity with a nucleic acid sequence disclosed herein. Preferably, a variant or mutant nucleic acid sequence will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to a full-length nucleic acid sequence or a fragment of a nucleic acid sequence as described herein. Nucleic acid sequence identity is defined as the percentage of nucleic acids in the variant sequence that are identical with the nucleic acids in the reference sequence, after aligning the sequences and introducing gaps, if necessary, to achieve the maximum percent sequence identity, and not considering any conservative substitutions as part of the sequence identity. Standard alignment algorithms cited above are useful in this regard.

According to a preferred embodiment, an isolated polypeptide according to the invention may be a polypeptide in which the serine protease activity is inactivated by amino acid substitution in the catalytic triad or in the serine protease motif.

According to another embodiment, the present invention relates to a nucleic acid encoding a polypeptide according to the invention.

According to another embodiment, the present invention relates to a vector comprising a nucleic acid according to the invention, optionally an expression vector.

According to another embodiment, the present invention relates to a host cell comprising a nucleic acid according to the invention or a vector according to the invention.

According to another embodiment, the present invention relates to a polypeptide according to the invention for use as a vaccine, in particular for use for the prevention or the treatment of N. meningitidis B infection.

According to another embodiment, the present invention relates to a vaccine composition comprising a polypeptide according to the invention.

According to another embodiment, the present invention relates to a method of production of a polypeptide according to the invention, the method comprising expression of said polypeptide from a vector according to the invention.

Fragment Polypeptides

Advantageously, when the fragment polypeptide comprises or consists of a fragment essentially consisting of the protease domain, the α-peptide domain and part of the beta-domain e.g., comprising at least one and no more than eleven beta-sheets of the trypsin-like serine protease auto-transporter of N. meningitidis, part of the beta-domain may comprise at least one and no more than eight, six, four or preferably, two beta-sheets. Part of the beta-domain may comprise from N-ter to C-ter, at least the first beta-sheet; (ii) first and second beta-sheets; (iii) first, second and third beta-sheets; (iv) first, second, third and fourth beta-sheets; (v) first, second, third, fourth and fifth beta-sheets; (vi) first, second, third, fourth, fifth and sixth beta-sheets; (viii) first, second, third, fourth, fifth, sixth and seventh beta-sheets; or (viii) first, second, third, fourth, fifth, sixth, seventh, and eighth beta-sheets; option (ii) being preferred.

According to one embodiment, an isolated polypeptide in accordance with the invention may consist of the protease domain of the trypsin-like serine protease auto-transporter of N. meningitidis which is IgA1P, App or AusI, and preferably is IgA1P.

According to one embodiment, an isolated polypeptide may consist of a protease domain and all or part of an α-peptide domain, and preferably of a passenger domain (protease domain and α-peptide domain), of the trypsin-like serine protease auto-transporter of N. meningitidis which is IgA1P, App or AusI.

According to one embodiment, an isolated polypeptide in accordance with the invention may consist of the protease domain, the α-peptide domain (together the passenger domain) and a part of a β-domain, preferably the two first β-sheets of the β-domain, of the trypsin-like serine protease auto-transporter IgA1P.

According to one embodiment, an isolated polypeptide in accordance with the invention may consist of the protease domain, the α-peptide domain (together the passenger domain) and a part of a β-domain, preferably the two first β-sheets of the β-domain, of the trypsin-like serine protease auto-transporter App.

According to one embodiment, an isolated polypeptide in accordance with the invention may consist of the protease domain, the α-peptide domain (together the passenger domain) and a part of a β-domain, preferably the two first β-sheets of the β-domain, of the trypsin-like serine protease auto-transporter AusI.

According to one embodiment, an isolated polypeptide in accordance with the invention may have an amino acid sequence having at least 90% identity with the amino acid sequence of the IgA1P of N. meningitidis MC58 shown in SEQ ID NO: 1 starting from position 27, 28, 29, 30, 31 or 32 and ending at a position selected from position 1008 to position 1505.

According to one embodiment, an isolated polypeptide in accordance with the invention may have an amino acid sequence having at least 90% identity with the amino acid sequence of the IgA1P of N. meningitidis MC58 shown in SEQ ID NO: 1 starting from position 27, 28, 29, 30, 31 or 32 and ending at position 1002, 1003, 1004, 1005, 1006, 1007 or 1008.

According to another embodiment, an isolated polypeptide in accordance with the invention may have an amino acid sequence having at least 90% identity with the amino acid sequence of the IgA1P of N. meningitidis MC58 shown in SEQ ID NO 1 starting from position 27, 28, 29, 30, 31 or 32 and ending at a position selected from position 1500, 1501, 1502, 1503, 1504, 1505, 1506, 1507, 1508, 1509 and 1510.

According to another embodiment, an isolated polypeptide in accordance with the invention may have an amino acid sequence having at least 90% identity with the amino acid sequence of the IgA1P of N. meningitidis MC58 shown in SEQ ID NO: 1 starting from position 27, 28, 29, 30, 31 or 32 and ending at position 1580, 1581, 1582, 1583, 1584, 1585, 1586, 1587 or 1588.

As a matter of example, an IgA1P fragment may essentially consist of the protease domain of MC58 IgA1P comprising (having) the amino acid sequence shown in SEQ ID NO: 1:

(i) starting with the amino acid in position 27 or 28 and ending with amino acid in position 1005; or

(ii) starting with the amino acid in position 27, 28, 29, 30, 31 or 32 and ending with the amino acid in position 1002, 1003, 1004, 1005, 1006, 1007, or 1008; or

(iii) starting with an amino acid in any one of positions 27 or 28 to 78, preferably 27 or 28 to 58, more preferably 27 or 28 to 38, and ending with an amino acid in any one of positions 990 to 1015, preferably 1000 to 1010, e.g., in position 1005.

Another example of an IgA1P fragment may essentially consist of the protease domain of a variant of MC58 IgA1P, said fragment being described by reference to the MC58 amino acid sequence reported in SEQ ID NO: 1 as comprising (having) an amino acid sequence:

(i) starting with the amino acid corresponding to the amino acid in position 27 or 28 and ending with the amino acid corresponding to the amino acid in position 1005; or

(ii) starting with the amino acid corresponding to the amino acid in position 27, 28, 29, 30, 31 or 32 and ending with the amino acid corresponding to the amino acid in position 1002, 1003, 1004, 1005, 1006, 1007, or 1008; or

(iii) starting with the amino acid corresponding to the amino acid in any one of the positions 28 to 78, preferably 28 to 58, more preferably 28 to 38, and ending with the amino acid corresponding to the amino acid in any one of positions 990 to 1015, preferably 1000 to 1010, e.g., in position 1005, in SEQ ID NO: 1.

Still as a matter of example, an IgA1P fragment may also be an MC58 IgA1P fragment essentially consisting of the IgA1P protease domain and all or part of the α-peptide domain and e.g., comprising (having) the amino acid sequence shown in SEQ ID NO: 1:

(i) starting with the amino acid in position 27 or 28 and ending with the amino acid in any one of positions 1005 to 1510, preferably 1500 to 1510, more preferably in position 1505; or

(ii) starting with the amino acid in any one of positions 27 or 28 to 78, preferably 27 or 28 to 58, more preferably 27 or 28 to 38, and ending with the amino acid in any one of positions 1005 to 1510, preferably 1300 or 1500 to 1510, e.g. in position 1505.

Another example of an IgA1P fragment may be a fragment of a variant of MC58 IgA1P, said fragment essentially consisting of the IgA1P protease domain and all or part of the α-peptide domain and being described by reference to the MC58 amino acid sequence reported in SEQ ID NO: 1, which comprises (has) an amino acid sequence:

(i) starting with the amino acid corresponding to the amino acid in position 27 or 28 and ending with the amino acid corresponding to the amino acid in any one of positions 1005 to 1510, preferably 1500 to 1510, more preferably in position 1505; or

(ii) starting with the amino acid corresponding to the amino acid in any one of positions 27 or 28 to 78, preferably 27 or 28 to 58, more preferably 27 or 28 to 38, and ending with the amino acid corresponding to the amino acid in any one of positions 1005 to 1510, preferably 1300 or 1500 to 1510, e.g., in position 1505, in SEQ ID NO: 1.

Still as a matter of example, an IgA1P fragment may also be the MC58 IgA1P fragment essentially consisting of the IgA1P protease domain, the α-peptide domain and part of the beta-domain e.g. comprising at least one and no more than eleven beta sheets and e.g., comprising (having) the amino acid sequence shown in SEQ ID NO: 1:

(i) starting with the amino acid in position 27 or 28 and ending with the amino acid in position 1584; or

(ii) starting with the amino acid in any one of positions 27 or 28 to 78, preferably 27 or 28 to 58, more preferably 27 or 28 to 38, and ending with the amino acid in any one of positions 1505 to 1600, preferably 1550 to 1590, e.g. in position 1584.

Another example of an IgA1P fragment may be a fragment of a variant of MC58 IgA1P, said fragment essentially consisting of the IgA1P protease domain, the α-peptide domain and part of the beta-domain e.g. comprising at least one and no more than eleven beta-sheets and being described by reference to the MC58 amino acid sequence reported in SEQ ID NO: 1, which comprises (has) an amino acid sequence:

(i) starting with the amino acid corresponding to the amino acid in position 27 or 28 and ending with the amino acid corresponding to the amino acid in position 1584; or

(ii) starting with the amino acid corresponding to the amino acid in any one of positions 27 or 28 to 78, preferably 27 or 28 to 58, more preferably 27 or 28 to 38, and ending with the amino acid corresponding to the amino acid in any one of positions 1505 to 1600, preferably 1550 to 1590, e.g., in position 1584, in SEQ ID NO: 1.

According to a preferred embodiment, an isolated polypeptide in accordance with the invention may have an amino acid sequence having at least 90% identity with, and preferably may consist in, the amino acid sequence of the IgA1P of N. meningitidis MC58 shown in SEQ ID NO: 1 starting from position 26, 27, 28, 29 or 30, an preferably 28, and ending at position 1582, 1583, 1584, 1585 or 1586, and preferably 1584.

According to another preferred embodiment, an isolated polypeptide in accordance with the invention may have an amino acid sequence having at least 90% identity with, and preferably may consist in, the amino acid sequence of the IgA1P of N. meningitidis MC58 shown in SEQ ID NO: 1 starting from position 26, 27, 28, 29 or 30, an preferably 28, and ending at position 1003, 1004, 1005, 1006 or 1007, and preferably 1005.

The polypeptide in accordance with the invention, and in particular those preferred embodiments may further comprise a mutation in the catalytic site as described below to reduce or suppress the catalytic activity, preferably at the Serine in position 267. Preferably, the Serine may be change for a Valine.

According to one embodiment, an isolated polypeptide in accordance with the invention may have an amino acid sequence having at least 90% identity with the amino acid sequence of the App of N. meningitidis MC58 shown in SEQ ID NO: 3 starting from position 40, 41, 42, 43, 44, 45 or 46 and ending at a position selected from positions 1057 to position 1204.

According to one embodiment, an isolated polypeptide in accordance with the invention may have an amino acid sequence having at least 90% identity with the amino acid sequence of the App of N. meningitidis MC58 shown in SEQ ID NO: 3 starting from position 40, 41, 42, 43, 44, 45 or 46 and ending at position 1052, 1053, 1054, 1055, 1056, 1057, 1058, 1059 or 1060.

According to one embodiment, an isolated polypeptide in accordance with the invention may have an amino acid sequence having at least 90% identity with the amino acid sequence of the App of N. meningitidis MC58 shown in SEQ ID NO: 3 starting from position 40, 41, 42, 43, 44, 45 or 46 and ending at a position between 1170 and 1204 inclusive

According to one embodiment, an isolated polypeptide in accordance with the invention may have an amino acid sequence having at least 90% identity with the amino acid sequence of the App of N. meningitidis MC58 shown in SEQ ID NO: 3 starting from position 40, 41, 42, 43, 44, 45 or 46 and ending at position 1220, 1220, 1221, 1223, 1224, 1225, 1226 or 1227.

As a matter of example, an App fragment may essentially consist of the protease domain of:

(i) MC58 App comprising (having) the amino acid sequence shown in SEQ ID NO: 3 starting with the amino acid in position 40, 41, 42, 43, 44, 45 or 46 and ending with amino acid in position 1052, 1053, 1055, 1056, 1057, 1058, 1059 or 1060; or

(ii) a variant of MC58 App, said fragment being described by reference to the MC58 amino acid sequence reported in SEQ ID NO: 3 as comprising (having) an amino acid sequence starting with an amino acid corresponding to the amino acid in position 40, 41, 42, 43, 44, 45 or 46 and ending with the amino acid corresponding to the amino acid in position 1052, 1053, 1055, 1056, 1057, 1058, 1059 or 1060, in SEQ ID NO: 3.

Still as a matter of example, an App fragment may also be the MC58 App fragment essentially consisting of the App protease domain and all or part of the α-peptide domain and e.g., comprising (having) the amino acid sequence shown in SEQ ID NO: 3

(i) starting with the amino acid in position 42 or 43 and ending with the amino acid in position 1175 or 1187; or

(ii) starting with the amino acid in any one of positions 42 or 43 to 92, preferably 42 or 43 to 72, more preferably 42 or 43 to 52, and ending with the amino acid in any one of positions 1060 or 1160 to 1210, preferably 1170 to 1200, e.g., in position 1175 or 1187.

Another example of an App fragment may be a fragment of a variant of MC58 App, said fragment essentially consisting of the App protease domain and all or part of the α-peptide domain and being described by reference to the MC58 amino acid sequence reported in SEQ ID NO: 3, which comprises (has) an amino acid sequence (i) starting with the amino acid corresponding to the amino acid in position 42 or 43 and ending with the amino acid corresponding to the amino acid in position 1175 or 1187; or

(ii) starting with the amino acid corresponding to the amino acid in any one of the positions 42 or 43 to 92, preferably 42 or 43 to 72, more preferably 42 or 43 to 52, and ending with the amino acid corresponding to the amino acid in any one of positions 1060 or 1160 to 1210, preferably 1170 to 1200, e.g., in position 1175 or 1187, in SEQ ID NO: 3.

Still as a matter of example, an App fragment may also be the MC58 App fragment essentially consisting of the App protease domain, the α-peptide domain and part of the beta and e.g., comprising (having) the amino acid sequence shown in SEQ ID NO: 3

(i) starting with the amino acid in position 42 or 43 and ending with the amino acid in position 1224; or

(ii) starting with the amino acid in any one of positions 42 or 43 to 92, preferably 42 or 43 to 72, more preferably 42 or 43 to 52, and ending with the amino acid in any one of positions 1175 to 1240, preferably 1210 to 1230, e.g., in position 1224.

Another example of an App may be a fragment of a variant of MC58 App, said fragment essentially consisting of the App protease domain, the α-peptide domain and part of the beta domain and being described by reference to the MC58 amino acid sequence reported in SEQ ID NO: 3, which comprises (has) an amino acid sequence

(i) starting with the amino acid corresponding to the amino acid in position 42 or 43 and ending with the amino acid corresponding to the amino acid in position 1224; or

(ii) starting with the amino acid corresponding to the amino acid in any one of the positions 42 or 43 to 92, preferably 42 or 43 to 72, more preferably 42 or 43 to 52, and ending with the amino acid corresponding to the amino acid in any one of positions 1175 to 1240, preferably 1210 to 1230, e.g., in position 1224, in SEQ ID NO: 3.

According to a preferred embodiment, an isolated polypeptide in accordance with the invention may have an amino acid sequence having at least 90% identity with, and preferably may consist in, the amino acid sequence of the App of N. meningitidis MC58 shown in SEQ ID NO: 3 starting from position 41, 42, 43, 44 or 45, and preferably 43, and ending at position 1122, 1123, 1224, 1225 or 1126, and preferably 1224.

The polypeptide in accordance with the invention, and in particular those preferred embodiments may further comprise a mutation in the catalytic site as described below to reduce or suppress the catalytic activity, preferably at the Serine in position 267. Preferably, the Serine may be change for a Valine.

According to one embodiment, an isolated polypeptide in accordance with the invention may have an amino acid sequence having at least 90% identity with the amino acid sequence of the AusI of N. meningitidis MC58 shown in SEQ ID NO: 5 starting from position 26, 27, 28, 29, 30 or 31 and ending at a position selected from position 969 to position 1177.

According to one embodiment, an isolated polypeptide in accordance with the invention may have an amino acid sequence having at least 90% identity with the amino acid sequence of the AusI of N. meningitidis MC58 shown in SEQ ID NO: 5 starting from position 26, 27, 28, 29, 30 or 31 and ending at position 966, 967, 968, 969, 970, 971 or 972.

According to one embodiment, an isolated polypeptide in accordance with the invention may have an amino acid sequence having at least 90% identity with the amino acid sequence of the AusI of N. meningitidis MC58 shown in SEQ ID NO: 5 starting from position 26, 27, 28, 29, 30 or 31 and ending at a position between 1131 and 1177 inclusive.

According to one embodiment, an isolated polypeptide in accordance with the invention may have an amino acid sequence having at least 90% identity with the amino acid sequence of the AusI of N. meningitidis MC58 shown in SEQ ID NO: 3 starting from position 26, 27, 28, 29, 30 or 31 and ending at position 1194, 1195, 1196, 1197, 1198, 1199, 1200, 1201 or 1202.

As a matter of example, an AusI fragment may essentially consist of the protease domain of:

(i) MC58 AusI comprising (having) the amino acid sequence shown in SEQ ID NO: 5 starting with the amino acid in position 26, 27, 28, 29, 30 or 31 and ending with amino acid in position 965, 966, 967, 968, 969, 970, 971 or 972; or

(ii) a variant of MC58 AusI, said fragment being described by reference to the MC58 amino acid sequence reported in SEQ ID NO: 5 as comprising (having) an amino acid sequence starting with an amino acid corresponding to the amino acid in position 26, 27, 28, 29, 30 or 31 and ending with the amino acid corresponding to the amino acid in position 965, 966, 967, 968, 969, 970, 971 or 972.

Still as a matter of example, an AusI fragment may be the MC58 AusI fragment essentially consisting of the AusI protease domain and all or part of the α-peptide domain and e.g., comprising (having) the amino acid sequence shown in SEQ ID NO: 5

(i) starting with the amino acid in position 26 or 27 and ending with the amino acid in any one of the positions 1130 to 1180, e.g., in position 1161; or

(ii) starting with the amino acid in any one of positions 26 or 27 to 76, preferably 26 or 27 to 56, more preferably 26 or 27 to 36, and ending with the amino acid in any one of positions 972 or 1130 to 1180, e.g., in position 1161

Another example of an AusI fragment may be a fragment of a variant of MC58 AusI, said fragment essentially consisting of the AusI protease domain and all or part of the α-peptide domain and being described by reference to the MC58 amino acid sequence reported in SEQ ID NO: 5, which comprises (has) an amino acid sequence

(i) starting with the amino acid corresponding to the amino acid in position 26 or 27 and ending with the amino acid corresponding to the amino acid in any one of the positions 1130 to 1180, e.g., in position 1161; or

(ii) starting with the amino acid corresponding to the amino acid in any one of the positions 26 or 27 to 76, preferably 26 or 27 to 56, more preferably 26 or 27 to 36, and ending with the amino acid corresponding to the amino acid in any one of positions 972 or 1130 to 1180, e.g., in position 1161, in SEQ ID N05.

Still as a matter of example, an AusI fragment may be the MC58 AusI fragment essentially consisting of the AusI protease domain, the α-peptide domain and part of the beta-domain and e.g., comprising (having) the amino acid sequence shown in SEQ ID NO: 5:

(i) starting with the amino acid in position 26 or 27 and ending with the amino acid in any one of the positions 1198; or

(ii) starting with the amino acid in any one of positions 26 or 27 to 76, preferably 26 or 27 to 56, more preferably 26 or 27 to 36, and ending with the amino acid in any one of positions 1130 or 1180 to 1210, preferably 1190 to 1200, e.g., in position 1198.

Another example of an AusI fragment may be a fragment of a variant of MC58 AusI, said fragment essentially consisting of the AusI protease domain, the α-peptide domain and part of the beta-domain and being described by reference to the MC58 amino acid sequence reported in SEQ ID NO: 5, which comprises (has) an amino acid sequence:

(i) starting with the amino acid corresponding to the amino acid in position 26 or 27 and ending with the amino acid corresponding to the amino acid in any one of the positions 1130 to 1180; or

(ii) starting with the amino acid corresponding to the amino acid in any one of the positions 26 or 27 to 76, preferably 26 or 27 to 56, more preferably 26 or 27 to 36, and ending with the amino acid corresponding to the amino acid in any one positions 1130 or 1180 to 1210, preferably 1190 to 1200, e.g., in position 1198, in SEQ ID NO: 5.

According to a preferred embodiment, an isolated polypeptide in accordance with the invention may have an amino acid sequence having at least 90% identity with, and preferably may consist in, the amino acid sequence of the AusI of N. meningitidis MC58 shown in SEQ ID NO: 5 starting from position 24, 25, 26, 27 or 28, and preferably 26, and ending at position 1196, 1197, 1198, 1199 or 1200, and preferably 1198.

The polypeptide in accordance with the invention, and in particular those preferred embodiments may further comprise a mutation in the catalytic site as described below to reduce or suppress the catalytic activity, preferably at the Serine in position 241. Preferably, the Serine may be change for a Valine.

As already mentioned above, in some embodiments, the fragment polypeptide of the invention i.a., such as described above, may be mutated so that it lacks or has reduced trypsin-like serine protease activity and/or does not contain any cleavage site susceptible/able to be cleaved by a trypsin-like serine protease. As a result of the mutation, the auto-transporter remains in a precursor state, the N-terminal protease sub-domain not being cleaved from the rest of the molecule. For example, protease activity may be reduced by 10, 20, 30, 40, 50, 60, 70, 80, 90, 95, 96, 97, 98, 99 or 100% compared to the wild type sequence. Protease activity may be evaluated by assaying the ability to cleave auto-transporter proteins, for example by Western Blot as described in for example Roussel-Jazédé et al., Infect Immun. (2010) 78 (7): 3083; van Ulsen P. et al., Mol Microbiol. (November 2003) (3): 1017 and Serruto et al., PNAS February 2010 107 (8): 3770. Protease activity may also be assayed by, for example, the method described in Vitovski et al., (1999) FASEB J. 13: 331 for IgA1P. Preferably, the fragment polypeptide of the invention i.a., such as described above, lacks trypsin-like serine protease activity. As already mentioned above, the catalytic triad of the serine protease autotransporters responsible for the protease activity includes a Serine residue. In order to reduce or abolish the serine protease activity, any of the amino acids present in the catalytic triad (located in the protease sub-domain) may be mutated, advantageously by amino acid substitution. In a particular embodiment, one way to achieve that goal may be to substitute the Serine residue in the catalytic triad by any other amino acid, advantageously by Glycine, Threonine, Alanine, Leucine, Isoleucine or Valine, this latter amino acid being preferred.

Examples of useful mutated IgA1P App or AusI fragments include in particular any one of the MC58 IgA1P, App or AusI fragments or variants thereof as described above, each being mutated so that it lacks or has reduced trypsin-like serine protease activity and/or does not contain any cleavage site susceptible/able to be cleaved by a trypsin-like serine protease.

As already mentioned above, the catalytic triad of IgA1P from N. meningitidis strain MC58 of SEQ ID NO: 1 is generally considered to be 101H 150D 267S. The catalytic triad of App from N. meningitidis strain MC58 of SEQ ID NO: 3 is generally considered to be 115H 158D 267S. The catalytic triad of AusI from N. meningitidis strain MC58 of SEQ ID NO: 5 is generally considered to be 100H 135D 241S. The catalytic residues of proteins from other N. meningitidis strains may be determined by reference to the corresponding MC58 amino acid sequences as described herein, for example by reference to SEQ ID NO: 1 in the case of IgA1P.

The catalytic triad of MC58 IgA1P is composed of His101, Asp150 and Ser267 in SEQ ID NO: 1. Accordingly, the catalytic triad of MC58 IgA1P variants is composed of amino acids corresponding to His101, Asp150 and Ser267 in the amino acid sequence of SEQ ID NO: 1; and accordingly, the mutation occurs at the position corresponding to His101, Asp150 and Ser267 of the amino acid sequence of SEQ ID NO: 1. While any of the amino acids being or corresponding to His101, Asp150 and Ser267 of the amino acid sequence of SEQ ID NO: 1 may be mutated e.g. by substitution, it is preferred to substitute the serine residue in the catalytic triad by any other amino acid, advantageously by e.g. Glycine, Threonine, Alanine, Leucine, Isoleucine and Valine, this latter amino acid being preferred.

A useful mutation as described above may be introduced in any of the IgA1P fragment i.a., as described above. In particular, the mutation that may be introduced in MC58 IgA1P polypeptide sequence is S267V. In a similar manner, the mutation that may be introduced in MC58 variant polypeptide sequences is the substitution of the Serine residue in the catalytic triad with Valine.

As a matter of non-limiting illustration, particular examples include:
The S267V mutated MC58 IgA1P fragment which comprises (has) the amino acid sequence shown in SEQ ID NO: 1, starting with the amino acid in position 27 or 28, and ending with the amino acid in position 1005 or 1584; and in which Serine 267 is substituted by Valine; and
A mutated variant of the S267V MC58 IgA1P fragment which may be described as follows by reference to the MC58 amino acid sequence reported in SEQ ID NO: 1: this mutated variant comprises (has) an amino acid sequence starting with an amino acid corresponding to the amino acid in position 27 or 28, and ending with an amino acid corresponding to the amino acid in position 1005 or 1584 and in which the amino acid corresponding to Serine 267 is substituted by Valine.

In some embodiments, a fragment polypeptide comprises or consists of an IgA1P fragment which essentially consists of:

(i) the IgA1P protease domain in which the Ser residue of the catalytic triad is optionally mutated by substitution with e.g., Valine, the α-peptide domain and at least one and no more than eleven beta sheets, preferably the first and second beta sheets; or

(ii) the IgA1P protease domain in which the Ser residue of the catalytic triad is optionally mutated by substitution with e.g., Valine.

The catalytic triad of MC58 App is composed of His115, Asp158 and Ser267 in SEQ ID NO: 3. Accordingly, the catalytic triad of MC58 App variants is composed of amino acids corresponding to His115, Asp158 and Ser267 in the amino acid sequence of SEQ ID NO: 3; and accordingly, the mutation occurs at the position corresponding to His115, Asp158 and Ser267 of the amino acid sequence of SEQ ID NO: 3. While any of the amino acids being or corresponding to His115, Asp158 and Ser267 of the amino acid sequence of SEQ ID NO: 3 may be mutated e.g. by substitution, it is preferred to substitute the serine residue in the catalytic triad by any other amino acid, advantageously by e.g. Glycine, Threonine, Alanine, Leucine, Isoleucine and Valine, this latter amino acid being preferred.

A useful mutation as described above may be introduced in any of the App fragments thereof as described above. In particular, the mutation that may be introduced in MC58 App polypeptide sequence is S267V. In a similar manner, the mutation that may be introduced in MC58 variant App polypeptide sequences is the subsitution of the Serine residue in the catalytic triad with Valine.

As a matter of non-limiting illustration, particular examples include:
The S267V mutated MC58 App fragment which comprises (has) the amino acid sequence shown in SEQ ID NO: 3, starting with the amino acid in position 42 or 43, and ending with the amino acid in position 1224; and in which Serine 267 is substituted by Valine; and
A mutated variant of the S267V MC58 App fragment which may be described as follows by reference to the MC58 amino acid sequence reported in SEQ ID NO: 3: this mutated variant comprises (has) an amino acid sequence starting with an amino acid corresponding to the amino acid in position 42 or 43, and ending with an amino acid corresponding to the amino acid in position 1224 and in which the amino acid corresponding to Serine 267 is substituted by Valine.

The catalytic triad of MC58 AusI is composed of His100, Asp135 and Ser241 in SEQ ID NO: 5. Accordingly, the catalytic triad of MC58 AusI variants is composed of amino acids corresponding to His100, Asp135 and Ser241 in the amino acid sequence of SEQ ID NO: 5; and accordingly, the mutation occurs at the position corresponding to His100, Asp135 and Ser241 of the amino acid sequence of SEQ ID NO: 5. While any of the amino acids being or corresponding to His100, Asp135 and Ser241 of the amino acid sequence of SEQ ID NO: 5 may be mutated e.g. by substitution, it is preferred to substitute the serine residue in the catalytic triad by any other amino acid, advantageously by e.g. Glycine, Threonine, Alanine, Leucine, Isoleucine and Valine, this latter amino acid being preferred.

A useful mutation as described above may be introduced in any of the AusI fragments thereof as described above. In particular, the mutation that may be introduced in MC58 AusI polypeptide sequence is S241V. In a similar manner, the mutation that may be introduced in MC58 variant AusI polypeptide sequences is the substitution of the Serine residue in the catalytic triad with Valine.

As a matter of non-limiting illustration, particular examples include:
The S241V mutated MC58 AusI fragment which comprises (has) the amino acid sequence shown in SEQ ID NO: 5, starting with the amino acid in position 26 or 27 and ending with the amino acid in position 1198 and in which Serine 241 is substituted by Valine; and
A mutated variant of the S241V MC58 AusI fragment which may be described as follows by reference to the MC58 amino acid sequence reported in SEQ ID NO: 5: this mutated variant comprises (has) an amino acid sequence starting with an amino acid corresponding to the amino acid in position 26 or 27, and ending with an amino acid corresponding to the amino acid in position 1198 and in which the amino acid corresponding to the Serine 267 is substituted by Valine.

In other words, the IgA1P fragment polypeptide may comprise or consist of: An IgA1P fragment (i.e., a MC58 IgA1P fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 1, starting with the amino acid in position 27, 28, 29, 30, 31, or 32 and ending with the amino acid in position 1002, 1003, 1004, 1005, 1006, 1007 or 1008;

An IgA1P fragment (i.e., a MC58 IgA1P fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 1, starting with the amino acid in position 27, 28, 29, 30, 31, or 32 and ending with the amino acid at a position selected from positions 1008 to 1505 inclusive;

An IgA1P fragment (i.e., a MC58 IgA1P fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 1, starting with the amino acid in position 27, 28, 29, 30, 31, or 32 and ending with the amino acid in position 1500, 1501, 1502, 1503, 1504, 1505, 1506, 1507, 1508, 1509 or 1510;

An IgA1P fragment (i.e., a MC58 IgA1P fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 1, starting with the amino acid in position 27, 28, 29, 30, 31, or 32 and ending with the amino acid in any one of positions 1506 to 1700, preferably 1506 to 1600, more preferably 1550 to 1600; or

An IgA1P fragment (i.e., a MC58 IgA1P fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 1, starting with the amino acid in position 27, 28, 29, 30, 31, or 32 and ending with amino acid in position 1580, 1581, 1582, 1583, 1584, 1585, 1586, 1587, or 1588.

In other words, the App fragment polypeptide may comprise or consist of:

An App fragment (i.e., a MC58 App fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 40, 41, 42, 43, 44, 45 or 46 and ending with the amino acid in position 1052, 1053, 1054, 1055, 1056, 1057, 1058, 105 or 1060;

An App fragment (i.e., a MC58 App fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 3, starting with the amino acid in position 40, 41, 42, 43, 44, 45 or 46 and ending with the amino acid at a position selected from positions 1057 to 1204 inclusive;

An App fragment (i.e., a MC58 App fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 3, starting with the amino acid in position 40, 41, 42, 43, 44, 45 or 46 and ending with the amino acid at a position between 1170 and 1204 inclusive;

An App fragment (i.e., a MC58 App fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 3, starting with the amino acid in position 40, 41, 42, 43, 44, 45 or 46 and ending with the amino acid in any one of positions 1205 to 1400, preferably 1205 to 1300, more preferably 1220 to 1260; or

An App fragment (i.e., a MC58 App fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 3, starting with the amino acid in position 40, 41, 42, 43, 44, 45 or 46 and ending with amino acid in position 1220, 1221, 1222, 1223, 1224, 1225, 1226, or 1227.

In other words, the AusI fragment polypeptide may comprise or consist of: An AusI fragment (i.e., a MC58 AusI fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 26, 27, 28, 29, 30 or 31 and ending with amino acid in position 965, 966, 967, 968, 969, 970, 971 or 972;

An AusI fragment (i.e., a MC58 AusI fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 26, 27, 28, 29, 30 or 31 and ending with amino acid at a position selected from positions 969 to 1177;

An AusI fragment (i.e., a MC58 AusI fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 26, 27, 28, 29, 30 or 31 and ending with amino acid at a position between 1131 and 1177 inclusive;

An AusI fragment (i.e., a MC58 AusI fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 26, 27, 28, 29, 30 or 31 and ending with amino acid in any one of positions 1178 to 1300, preferably 1178 to 1250, more preferably 1180 to 1220; OR

An AusI fragment (i.e., a MC58 AusI fragment or a variant thereof) being optionally mutated as described above, which comprises (has) an amino acid sequence having at least 80%, 85%, 90%, 95%, 96%, 97%, 98%, 99% or 100% identity with the MC58 amino acid sequence reported in SEQ ID NO: 5, starting with the amino acid in position 26, 27, 28, 29, 30 or 31 and ending with amino acid in position 1220, 1195, 1196, 1197, 1198, 1199, 1200, or 1201.

Fusion Polypeptides

Advantageously, in the fusion polypeptide of the invention, the first fragment may essentially consist of (be) the protease domain or protease sub-domain, and preferably is a protease sub-domain, of the first trypsin-like serine protease auto-transporter. In an advantageous and independent manner, the second fragment may essentially consist of (be) the α-peptide domain and optionally part of the β-domain of the second trypsin-like serine protease auto-transporter.

In some embodiments, in the fusion polypeptide, (i) the first fragment essentially consists of (is) the protease domain or protease sub-domain of the first trypsin-like serine protease auto-transporter and (ii) the second fragment essentially consists of the α-peptide domain and part of the β-domain of the second trypsin-like serine protease auto-transporter.

According to one embodiment, a polypeptide of the invention may comprise or consist of a first fragment fused to a second fragment wherein said first fragment consists of a protease sub-domain of said first trypsin-like serine protease auto-transporter and said second fragment consist of an α-peptide domain, optionally with a part of a β-domain, of said second trypsin-like serine protease auto-transporter.

According to a preferred embodiment, a polypeptide of the invention may comprise or consist of a first fragment fused to a second fragment wherein said first fragment consists of a protease sub-domain of said first trypsin-like serine protease auto-transporter which is IgA1P, and said second fragment consist of an α-peptide domain, optionally with a part of a β-domain, of said second trypsin-like serine protease auto-transporter which is App or AusI.

In a particular embodiment, part of the β-domain of the second fragment useful in the fusion polypeptide comprises at least one and no more than eleven β-sheets; preferably from two to eight β-sheets, more preferably from two to four β-sheets, most preferably two β-sheets. In practice, the C-terminus of the α-peptide domain is fused to the N-terminus of the β-domain which comprises from N-ter to C-ter, at least the first beta-sheet; (ii) first and second beta-sheets; (iii) first, second and third beta-sheets; (iv) first, second, third and fourth beta-sheets; (v) first, second, third, fourth and fifth beta-sheets; (vi) first, second, third, fourth, fifth and sixth beta-sheets; (viii) first, second, third, fourth, fifth, sixth and seventh beta-sheets; or (viii) first, second, third, fourth, fifth, sixth, seventh, and eighth beta-sheets; option (ii) being preferred.

For use in the fusion polypeptide, the first fragment may be mutated in the catalytic triad as described above with respect to the full-length mature trypsin-like serine protease auto-transporter of N. meningitidis, such as IgA1P, App or AusI, or the described fragments thereof. Indeed, the catalytic triad is located in the protease sub-domain. It may also be not mutated, especially when this first fragment essentially consists of the protease sub-domain that is the protease domain, lacking the C-terminus amino acids containing the auto-cleavage site.

The first and second fragments may independently be a trypsin-like serine protease fragment of an MC58 strain or a variant thereof.

According to one embodiment, an isolated peptide in accordance with the invention comprising or consisting of a first fragment fused to a second fragment may comprise or consist of a first fragment having at least 90% identity with an amino acid sequence of the IgA1P of N. meningitidis MC58 shown in SEQ ID NO: 1 starting from position 27, 28, 29, 30, 31 or 32 and ending at position 1002, 1003, 1004, 1005, 1006, 1007 or 1008;

and may comprise or consist of a second fragment having at least 90% identity with an amino acid sequence of

(i) App of N. meningitidis MC58 shown in SEQ ID NO: 3 starting from position 1057, 1058, 1059, 1060, 1061 or 1062 and ending at a position between 1170 and 1204 inclusive; or

(ii) AusI of N. meningitidis MC58 shown in SEQ ID NO: 3 starting from position 969, 970, 971, 972, 973, 974, 975, 976, 977, 978, 979 or 980 and ending at a position between 1131-1177 inclusive.

According to another embodiment, an isolated peptide in accordance with the invention comprising or consisting of a first fragment fused to a second fragment may comprise or consist of a first fragment having at least 90% identity with an amino acid sequence of the IgA1P of N. meningitidis MC58 shown in SEQ ID NO: 1 starting from position 27, 28, 29, 30, 31 or 32 and ending at position 960, 961, 962, 963, 964, 965, 966, 967, 968, 969, 9701, 971, 972, 973, 974, 975, 976, 977, 978, 979 or 980;

and may comprise or consist of a second fragment having at least 90% identity with an amino acid sequence of

(i) App of N. meningitidis MC58 shown in SEQ ID NO: 3 starting from position 1057, 1058, 1059, 1060, 1061 or 1062 and ending at a position between 1170 and 1204 inclusive; or

(ii) AusI of N. meningitidis MC58 shown in SEQ ID NO: 5 starting from position 969, 970, 971, 972, 973, 974, 975, 976, 977, 978, 979 or 980 and ending at a position between 1131 and 1177 inclusive.

According to another embodiment, an isolated peptide in accordance with the invention comprising or consisting of a first fragment fused to a second fragment may comprise or consist of a first fragment having at least 90% identity with an amino acid sequence of the IgA1P of N. meningitidis MC58 shown in SEQ ID NO: 1 starting from position 27, 28, 29, 30, 31 or 32 and ending at position 960, 961, 962, 963, 964, 965, 966, 967, 968, 969, 9701, 971, 972, 973, 974, 975, 976, 977, 978, 979 or 980;

and may comprise or consist of a second fragment having at least 90% identity with an amino acid sequence of

(i) App of N. meningitidis MC58 shown in SEQ ID NO 3: starting from position 1057, 1058, 1059, 1060, 1061 or 1062 and ending at a position between 1170 and 1204 inclusive, and preferably at a position 1183, 1184, 1185, 1186, 1187, 1188, 1189, 1190 or 1191; or

(ii) App of N. meningitidis MC58 shown in SEQ ID NO 3: starting from position 1057, 1058, 1059, 1060, 1061 or 1062 and ending at a 1220, 1221, 1222, 1223, 1224, 1125, 1226, 1227, or 1228; or

(iii) AusI of N. meningitidis MC58 shown in SEQ ID NO 5: starting from position 969, 970, 971, 972, 973, 974, 975, 976, 977, 978, 979 or 980 and ending at a position between 1131 and 1177 inclusive, and preferably at a position 1157, 1158, 1159, 1160, 1161, 1162, 1163, 1164, or 1165; or

(iv) AusI of N. meningitidis MC58 shown in SEQ ID NO 5: starting from position 969, 970, 971, 972, 973, 974, 975, 976, 977, 978, 979 or 980 and ending at a position between 1194, 1195, 1196, 1197, 1198, 1199, 1200, 1201 or 1202.

In a particular embodiment, the above constructs may further comprise a mutation in the catalytic site as previously described to reduce or suppress the catalytic activity. The mutation may in particular intervene at the Serine in position 267, which may, for instance, be replaced with a Valine.

As a matter of non-limiting illustration, the first fragment may be e.g. the MC58 IgA1P protease sub-domain and may be fused to the alpha-peptide domain of e.g. App of a variant of the MC58 strain.

In some embodiments, the first fragment in the fusion polypeptide essentially consists of the protease domain or the protease sub-domain of IgA1P, mutated or not as described above, and is fused to the second fragment which essentially consists of the α-peptide domain and optionally, part of the β-domain of App or AusI.

A particular example of these embodiments is an IgA1P-App fusion polypeptide which essentially consists of the IgA1P protease sub-domain in which the Ser residue of the catalytic triad is optionally mutated by substitution with e.g., Valine, fused to the App α-peptide domain.

As a matter of example, the fusion polypeptide comprises or consists of the protease sub-domain or protease domain of MC58 IgA1P, optionally bearing a mutation in the catalytic triad, fused to the alpha-peptide domain of MC58 App or AusI. Accordingly, such an MC58 fusion polypeptide may comprise or consist of:
(i) a protease sub-domain of MC58 IgA1P comprising (having) the amino acid sequence shown in SEQ ID NO: 1, optionally bearing the S267V mutation,
   starting with the amino acid in position 27 or 28 and ending with the amino acid in position 966; or
   starting with the amino acid in position 27, 28, 29, 30, 31 or 32 and ending with the amino acid in position 963, 964, 965, 966, 967, 968, or 969; or
   starting with the amino acid in any one of positions 27 or 28 to 78, preferably 27 or 28 to 58, more preferably 27 or 28 to 38, and ending with the amino acid in any one of positions 960 to 975, preferably 965 to 970, e.g., in position 966; or
(ii) a protease domain of MC58 IgA1P comprising (having) the amino acid sequence shown in SEQ ID NO: 1, optionally bearing the S267V mutation,
   starting with the amino acid in position 27 or 28 and ending with the amino acid in position 1005; or
   starting with the amino acid in position 27, 28, 29, 30, 31 or 32 and ending with the amino acid in position 1002, 1003, 1004, 1005, 1006, 1007 or 1008; or
   starting with the amino acid in any one of positions 27 or 28 to 78, preferably 27 or 28 to 58, more preferably 27 or 28 to 38, and ending with the amino acid in any one of positions 990 to 1015, preferably 1000 to 1010, e.g. in position 1005;
      fused to:
(i) the alpha-peptide domain of MC58 App comprising (having) the amino acid sequence shown in SEQ ID NO: 3 starting with the amino acid in any one of positions 1050 to 1070, preferably 1055 to 1065, e.g. in position 1055, 1056, 1057, 1058, 1059, 1060 or 1061 and ending with the amino acid in any one of positions 1160 to 1210, preferably 1170 to 1210, more preferably in position 1175 or 1187; or
(ii) the alpha-peptide domain of MC58 AusI comprising (having) the amino acid sequence shown in SEQ ID NO: 5 starting with the amino acid in any one of positions 965 to 980, preferably 969 to 975, e.g., in position 974 and ending with the amino acid in any one of positions 1130 to 1180, preferably 1155 to 1165, e.g., in position 1161.

Another example of a fusion polypeptide may be a fusion polypeptide which may be described by reference to the MC58 amino acid sequences reported in SEQ ID NO: 1, 3 and/or 5 as comprising or consisting of:
(i) the protease sub-domain of a variant of MC58 IgA1P comprising (having) the amino acid sequence described by reference to the amino acid sequence of SEQ ID NO: 1, optionally bearing the S267V mutation,
   starting with the amino acid corresponding to the amino acid in position 27 or 28 and ending with the amino acid corresponding to the amino acid in position 966; or
   starting with the amino acid corresponding the amino acid in position 27, 28, 29, 30, 31 or 32 and ending with the amino acid corresponding the amino acid in position 963, 964, 965, 966, 967, 968, or 969; or
   starting with the amino acid corresponding the amino acid in any one of positions 27 or 28 to 78, preferably 27 or 28 to 58, more preferably 27 or 28 to 38, and ending with the amino acid corresponding the amino acid in any one of positions 960 to 975, preferably 965 to 970, e.g., in position 966; OR
(ii) the protease domain of a variant of MC58 IgA1P comprising (having) an amino acid sequence described by reference to the amino acid sequence of SEQ ID NO: 1, optionally bearing the S267V mutation,
   starting with the amino acid corresponding to the amino acid in position 27 or 28 and ending with the amino acid corresponding to the amino acid in position 1005; or
   starting with the amino acid corresponding to the amino acid in position 27, 28, 29, 30, 31 or 32 and ending with the amino acid corresponding to the amino acid in position 1002, 1003, 1004, 1005, 1006, 1007 or 1008; or
   starting with the amino acid corresponding to the amino acid in any one of positions 27 or 28 to 78, preferably 27 or 28 to 58, more preferably 27 or 28 to 38, and ending with the amino acid corresponding to the amino acid in any one of positions 990 to 1015, preferably 1000 to 1010, e.g. in position 1005;
      fused to:
(i) the alpha-peptide domain of a variant of MC58 App comprising (having) an amino acid sequence described by reference to the amino acid sequence shown in SEQ ID NO: 3 starting with the amino acid corresponding to the amino acid in any one of positions 1050 to 1070, preferably 1055 to 1065, e.g. in position 1055, 1056, 1057, 1058, 1059, 1060 or 1061 and ending with the amino acid corresponding to the amino acid in any one of positions 1160 to 1210, preferably 1170 to 1210, more preferably in position 1175 or 1187; or
(ii) the alpha-peptide domain of a variant of MC58 AusI comprising (having) an amino acid sequence described by reference to the amino acid sequence shown in SEQ ID NO: 5 starting with the amino acid corresponding to the amino acid in any one of positions 965 to 980, preferably 969 to 975, e.g., in position 974 and ending with the amino acid corresponding to the amino acid in any one of positions 1130 to 1180, preferably 1155 to 1165, e.g., in position 1161.

Specific non-limiting examples include i.a.:

an MC58 fusion polypeptide comprising or consisting of a first fragment comprising (having) the amino acid sequence shown in SEQ ID NO: 1 starting with the amino acid in position 27 or 28 and ending with amino acid in position 966, optionally bearing the S267V mutation; fused to a second fragment comprising (having) the amino acid sequence shown in:
(i) SEQ ID NO: 3 starting with the amino acid in position 1061 and ending with the amino acid in position 1187; or
(ii) SEQ ID NO: 5 starting with the amino acid in position 974 and ending with the amino acid in position 1161.

A fusion polypeptide comprising or consisting of a first fragment comprising (having) an amino acid sequence described by reference to the amino acid sequence shown in SEQ ID NO: 1, optionally bearing a mutation corresponding to the S267V mutation, starting with the amino acid corresponding to the amino acid in position 27 or 28 and ending with the amino acid corresponding to the amino acid in position 966; fused to a second fragment comprising (having) an amino acid sequence described by reference to the amino acid sequence shown in:
(i) SEQ ID NO: 3 starting with the amino acid corresponding to the amino acid in position 1061 and ending with the amino acid corresponding to the amino acid in position 1187; or
(ii) SEQ ID NO: 5 starting with the amino acid corresponding to the amino acid in position 974 and ending with the amino acid corresponding to the amino acid in any one of positions ending with the amino acid in position 1161.

In some other embodiments, the first fragment in the fusion polypeptide essentially consists of the protease domain or the protease sub-domain of App, mutated or not as described above, and is fused to the second fragment which essentially consists of the α-peptide domain and optionally of part of the β-domain of IgA1P or AusI.

Still in some other embodiments, the first fragment in the fusion polypeptide essentially consists of the protease domain or the protease sub-domain of AusI, mutated or not as described above, and is fused to the second fragment which essentially consists of the α-peptide domain and optionally of part of the β-domain of App or IgA1P.

In some embodiments, the fusion polypeptide has first and second amino acid sequences, the C-terminus of the first sequence being fused to the N-terminus of the second sequence,

wherein the first sequence has at least 90% identity with the amino acid sequence of the IgA1P of N. meningitidis MC58 shown in SEQ ID NO: 1 starting from position 27, 28, 29, 30, 31 or 32 and ending at position 1002, 1003, 1004, 1005, 1006, 1007 or 1008; and

wherein the second sequence has at least 90% identity with the amino acid sequence of:

(i) App of N. meningitidis MC58 shown in SEQ ID NO: 3 starting from position 1057, 1058, 1059, 1060, 1061 or 1062 and ending at a position between 1170 and 1204 inclusive, preferably position 1187; or

(ii) AusI of N. meningitidis MC58 shown in SEQ ID NO: 5 starting from position 969, 970, 971, 972, 973, 974, 975, 976, 977, 978, 979 or 980 and ending at a position between 1131-1177 inclusive, preferably position 1161; or

(iii) App of N. meningitidis MC58 shown in SEQ ID NO: 3 starting from position 1057, 1058, 1059, 1060, 1061 or 1062 and ending at a position 1220, 1221, 1222, 1223, 1224, 1225 and 1226, preferably position 1224; or

(iv) AusI of N. meningitidis MC58 shown in SEQ ID NO: 5 starting from position 969, 970, 971, 972, 973, 974, 975, 976, 977, 978, 979 or 980 and ending at a position 1195, 1196, 1197, 1198, 1199, 1200, 1201, preferably position 1198.

According to another embodiment, an isolated peptide in accordance with the invention comprising or consisting of a first fragment fused to a second fragment may comprise or consist of a first fragment having at least 90% identity with an amino acid sequence of the App of N. meningitidis MC58 shown in SEQ ID NO: 3 starting from position 40, 41, 42, 43, 44, 45 or 46 and ending at position 1052, 1053, 1054, 1055, 1056, 1057, 1058, 1059 or 1060;

and may comprise or consist of a second fragment having at least 90% identity with an amino acid sequence of

(i) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510; or

(ii) AusI of N. meningitidis MC58 shown in SEQ ID NO: 5 starting from position 969, 970, 971, 972, 973, 974, 975, 976, 977, 978, 979 or 980 and ending at a position between 1131-1177 inclusive.

In some embodiments, the fusion polypeptide has first and second amino acid sequences, the C-terminus of the first sequence being fused to the N-terminus of the second sequence,

wherein the first sequence has at least 90% identity with the amino acid sequence of the App of N. meningitidis MC58 shown in SEQ ID NO: 3 starting from position 40, 41, 42, 43, 44, 45 or 46 and ending at position 1052, 1053, 1054, 1055, 1056, 1057, 1058, 1059 or 1060; and

wherein the second sequence has at least 90% identity with the amino acid sequence of:

(i) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510; or

(ii) AusI of N. meningitidis MC58 shown in SEQ ID NO: 5 starting from position 969, 970, 971, 972, 973, 974, 975, 976, 977, 978, 979 or 980 and ending at a position between 1131-1177 inclusive, preferably position 1161; or

(iii) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1580, 1581, 1582, 1583, 1584, 1585, 1586, 1587, and 1588, preferably 1584; or

(iv) AusI of N. meningitidis MC58 shown in SEQ ID NO: 5 starting from position 969, 970, 971, 972, 973, 974, 975, 976, 977, 978, 979 or 980 and ending at a position 1195, 1196, 1197, 1198, 1199, 1200, 1201, preferably position 1198.

According to another embodiment, an isolated peptide in accordance with the invention comprising or consisting of a first fragment fused to a second fragment may comprise or consist of a first fragment having at least 90% identity with an amino acid sequence of the AusI of N. meningitidis MC58 shown in SEQ ID NO: 5 starting from position 26, 27, 28, 29, 30 or 31 and ending at position 966, 967, 968, 969, 970, 971 or 972;

and may comprise or consist of a second fragment having at least 90% identity with an amino acid sequence of

(i) App of N. meningitidis MC58 shown in SEQ ID NO: 3 starting from position 1057, 1058, 1059, 1060, 1061 or 1062 and ending at a position between 1170 and 1204 inclusive; or

(ii) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510.

In some embodiments, the fusion polypeptide has first and second amino acid sequences, the C-terminus of the first sequence being fused to the N-terminus of the second sequence,

wherein the first sequence has at least 90% identity with the amino acid sequence of the AusI of N. meningitidis MC58 shown in SEQ ID NO: 5 starting from position 26, 27, 28, 29, 30 or 31 and ending at position 966, 967, 968, 969, 970, 971 or 972; and wherein the second sequence has at least 90% identity with the amino acid sequence of:

(i) App of N. meningitidis MC58 shown in SEQ ID NO: 3 starting from position 1057, 1058, 1059, 1060, 1061 or 1062 and ending at a position between 1170 and 1204 inclusive, preferably position 1187; or

(ii) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510; or

(iii) App of N. meningitidis MC58 shown in SEQ ID NO: 3 starting from position 1057, 1058, 1059, 1060, 1061 or 1062 and ending at a position 1220, 1221, 1222, 1223, 1224, 1225 and 1226, preferably position 1224; or

(iv) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1580, 1581, 1582, 1583, 1584, 1585, 1586, 1587, and 1588, preferably 1584.

In some embodiments, the fusion polypeptide has first and second amino acid sequences, the C-terminus of the first sequence being fused to the N-terminus of the second sequence,

wherein the first sequence has at least 90% identity with the amino acid sequence of the IgA1P of N. meningitidis MC58 shown in SEQ ID NO: 1 starting from position 27, 28, 29, 30, 31 or 32 and ending at position 960, 961, 962, 963, 964, 965, 966, 967, 968, 969, 970, 971, 972, 973, 974, 975, 976, 977, 978, 979 or 980; and wherein the second sequence has at least 90% identity with the amino acid sequence of:

(i) App of N. meningitidis MC58 shown in SEQ ID NO: 3 starting from position 1057, 1058, 1059, 1060, 1061 or 1062 and ending at a position between 1170 and 1204 inclusive, preferably position 1187; or

(ii) AusI of N. meningitidis MC58 shown in SEQ ID NO: 5 starting from position 969, 970, 971, 972, 973, 974, 975, 976, 977, 978, 979 or 980 and ending at a position between 1131 and 1177 inclusive, preferably position 1161; or

(iii) App of N. meningitidis MC58 shown in SEQ ID NO: 3 starting from position 1057, 1058, 1059, 1060, 1061 or 1062 and ending at a position 1220, 1221, 1222, 1223, 1224, 1225 and 1226, preferably position 1224; or

(iv) AusI of N. meningitidis MC58 shown in SEQ ID NO: 5 starting from position 969, 970, 971, 972, 973, 974, 975, 976, 977, 978, 979 or 980 and ending at a position 1195, 1196, 1197, 1198, 1199, 1200, 1201, preferably position 1198.

In some embodiments, the fusion polypeptide has first and second amino acid sequences, the C-terminus of the first sequence being fused to the N-terminus of the second sequence,

wherein the first sequence has at least 90% identity with the amino acid sequence of the App of N. meningitidis MC58 shown in SEQ ID NO: 3 starting from position 40, 41, 42, 43, 44, 45 or 46 and ending at position 950, 951, 952, 953, 954, 956, 957, 958, 959 or 960; and

wherein the second sequence has at least 90% identity with the amino acid sequence of:

(i) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510; or

(ii) AusI of N. meningitidis MC58 shown in SEQ ID NO: 5 starting from position 969, 970, 971, 972, 973, 974, 975, 976, 977, 978, 979 or 980 and ending at a position between 1131 and 1177 inclusive, preferably position 1161;

(iii) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1580, 1581, 1582, 1583, 1584, 1585, 1586, 1587, and 1588, preferably 1584; or

(iv) AusI of N. meningitidis MC58 shown in SEQ ID NO: 5 starting from position 969, 970, 971, 972, 973, 974, 975, 976, 977, 978, 979 or 980 and ending at a position 1195, 1196, 1197, 1198, 1199, 1200, 1201, preferably position 1198.

In some embodiments, the fusion polypeptide has first and second amino acid sequences, the C-terminus of the first sequence being fused to the N-terminus of the second sequence,

wherein the first sequence has at least 90% identity with the amino acid sequence of the AusI of N. meningitidis MC58 shown in SEQ ID NO: 5 starting from position 26, 27, 28, 29, 30 or 31 and ending at position 860, 861, 862, 863, 864, 865, 866, 867, 868, 869, 870, 871, 872 or 873; and

wherein the second sequence has at least 90% identity with the amino acid sequence of:

(i) App of N. meningitidis MC58 shown in SEQ ID NO: 3 starting from position 1057, 1058, 1059, 1060, 1061 or 1062 and ending at position 1170-1204, preferably position 1187; or

(ii) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1500, 1501, 1502, 1503, 1504, 1505, 1506, 1507, 1508, 1509 and 1510; or

(iii) App of N. meningitidis MC58 shown in SEQ ID NO: 3 starting from position 1057, 1058, 1059, 1060, 1061 or 1062 and ending at a position 1220, 1221, 1222, 1223, 1224, 1225 and 1226, preferably 1224; or

(iv) IgA1P of N. meningitidis MC58 shown in SEQ ID NO: 1 starting from position 1000, 1001, 1002, 1003, 1004, 1005, 1006, 1007, 1008, 1009, 1110, 1111, 1112, 1113 or 1114 and ending at a position selected from position 1580, 1581, 1582, 1583, 1584, 1585, 1586, 1587, and 1588, preferably 1584.

‘At least 90% identity’ naturally encompasses at least 91, 92, 93, 94, 95, 96, 97, 98, 99 and/or 100% identity.

The “fusion polypeptides” in accordance with the invention, in particular as described above may further comprise a mutation in the catalytic site as previously described to reduce or suppress the catalytic activity. The mutation may in particular intervene at the Serine position, which may, for instance, be replaced with a Valine.

All reference to ‘comprising’ herein should also be understood as including ‘consisting of’ and ‘essentially consisting of’. All reference to ‘essentially consisting of’ herein should also be understood as including ‘consisting of’. As used herein, ‘consisting of’, in the context of a polypeptide, indicates that said polypeptide does not contain additional amino acid sequence other than the recited sequence. As used herein, ‘essentially consisting of’, in the context of a polypeptide, indicates that the polypeptide may contain additional amino acid sequence other than the recited sequence, for example 1, 2, 3, 4, 5, 6, 7, 8, 9 or 10 additional amino acids at the N and/or C-terminus.

Unless otherwise indicated, all the polypeptides/fragments/constructs/amino acid sequences are described throughout the specification from the N-terminus end to the C-terminus end. As a matter of example, a fragment described as consisting of the protease domain, the α-peptide domain and part of the beta-domain of the trypsin-like serine protease auto-transporter of N. meningitidis shall be understood as a fragment consisting of, from N-ter to C-ter, the protease domain, the α-peptide domain and part of the beta-domain, the C-ter of the protease domain being fused to the N-ter of the α-peptide domain, the C-ter of which being fused to the N-ter of ‘part of the beta-domain’. Fusion is conveniently achieved by covalent peptidic bound (amide linkage CO—NH).

The polypeptides of the invention may be synthetized by any method well-known from the skilled person. Such methods include biological production methods by recombinant technology and means. In particular, nucleotide sequences encoding the N. meningitidis IgA1P, App and AusI (genes iga, app and ausI) and corresponding amino acid sequences thereof may be retrieved from a number of bioinformatics websites such as the site of the European Bioinformatics Institute or the National Center for Biotechnology Information (US). As a matter of example, sequences of strain MC58 (in particular, the NMB0700, NMB1985, NMB 1998 sequences) may be retrieved from the Entrez Gene database of the NCBI (National Center for the Biotechnology Information) at http://www.ncbi.nlm.nih.gov under the accession number NC_003112.

Any desired encoding sequences may be conceived and designed by bioinformatics according to methods and software known in the art, such as the software pack Vector NTI of Invitrogen; chemically synthetized de novo; and finally cloned into expression vectors available in the art. Methods of purification that can be used are also well-known from the skilled person.

Accordingly, the invention also provides nucleic acids encoding the polypeptides of the invention. Also provided are vectors comprising said nucleic acids, e.g., DNA, for example expression vectors, and host cells comprising said nucleic acids and/or vectors.

Also provided is a method of production of a polypeptide as described herein, the method comprising expressing said polypeptide from a vector as described herein. In particular a method of producing a polypeptide of the invention, comprises culturing a host cell e.g., a bacterial strain transformed with a vector (i) comprising a nucleotide sequence e.g., a DNA sequence encoding said polypeptide and (ii) able to express said polypeptide.

Variant and mutant nucleic acid sequences e.g., DNA sequences, include sequences capable of specifically hybridizing to the nucleotide sequences of strain MC58 encoding a polypeptide described herein under moderate or high stringency conditions.

Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C.

### Immunogenic Composition and Administration

The invention also provides a composition comprising a polypeptide of the invention.

In a preferred embodiment, said composition is an immunogenic composition comprising i.a., an immunologically effective amount of a polypeptide of the invention. In a preferred embodiment, said composition is a pharmaceutical e.g., vaccine composition, comprising i.a., a pharmaceutically, prophylactically and/or therapeutically effective amount of a polypeptide of the invention, i.a., together with a pharmaceutically acceptable excipient, e.g. a diluent or carrier.

### Adjuvants

In a particular embodiment of the invention, the composition according to the invention comprises one or several adjuvant(s).

The term “adjuvant” as used herein denotes a product which, added to the content of an immunogenic composition, in particular to a vaccine, increases the intensity of the immune reaction induced in the mammalian host to which said composition is administered. An adjuvant may in particular increase the quantity/quality of specific antibodies e.g. bactericidal antibodies, which said host is capable of producing after administration of said composition and thus increases the efficiency of the immune response.

The adjuvant (s) that can be used in the context of the invention include adjuvants promoting a Th1 and/or Th2 immune response. Accordingly, for use in the composition of the invention, an adjuvant may be a Th1, Th2 or Th1/Th2 adjuvant. The meaning given to “Th1, Th2 or Th1/Th2 adjuvant” shall be the meaning commonly acknowledged by the scientific community. A Th1 adjuvant promotes an immune response characterized by the predominant production of IFN-γ and/or IL-2 cytokines. A Th2 adjuvant promotes an immune response characterized by the predominant production of e.g., IL-4, IL-5, IL-6 and/or IL-10 cytokines. A Th1/Th2 adjuvant favours a balanced cytokine production (balanced immune response).

Examples of adjuvants promoting a Th1-type immune response include but are not limited to agonists of Toll-like receptors (TLRs), in particular to agonists of TLR4, which may be formulated or not. Typical formulation of a TLR agonist such as a TLR4 agonist, include oil-in-water emulsions. LPS derivatives like 3-De-O-acylated Monophosphoryl Lipid A (3D-MPL) described in WO 94/00153 or a 3D-MPL derivative named RC-529 described in U.S. Pat. No. 6,113,918 are well known TLR4 agonists; Other TLR4 agonists which share structural similarity with monophosphoryl lipid A, referred to as aminoalkyl glucosaminide phosphates (AGPs), are described in U.S. Pat. No. 6,113,918, U.S. Pat. No. 6,303,347, and WO 98/50399. Other synthetic TLR4 agonists are described in US 2003/0153532. Among these synthetic agonists, reference is made of a chemical compound named as E6020 and referenced in the Chemical Abstract Services (CAS) registry as CAS Number 287180-63-6 as particularly suitable Th1-adjuvant in the context of the invention. The chemical formula of the disodic salt is C83H63N4O19P2, 2Na and the developed chemical formula is as follows:

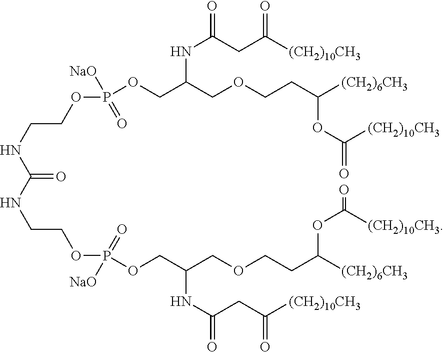

The R configuration (R,R,R,R) of the four asymetric carbons is preferred. The synthesis process is described in WO2007/005583. E6020 is preferably formulated in an oil-in-water emulsion and more particularly formulated in an oil-in-water emulsion (such as the one described in WO 07/006939), according to the process as described in the patent application WO 2007/080308.

Examples of adjuvants promoting a Th2-type immune response include but are not limited to aluminium salts and especially aluminium oxy hydroxide (also called for sake of brevity aluminium hydroxide) or aluminum hydroxy phosphate (also called for sake of brevity aluminum phosphate). When an aluminium salt is used, the protein antigens may advantageously be adsorbed onto the aluminium salt.

### Excipients

In a composition of the invention, the active ingredients may be formulated together with a pharmaceutically-acceptable excipient such as a pharmaceutically acceptable diluent or carrier. In a particular embodiment, the composition of the invention may comprise a buffer and/or an isotonic agent such as sodium chloride or sugars e.g. sucrose; and/or a stabilizing agent such as histidine.

An immunogenic composition according to the invention is useful for inducing an immune response in a mammal, in particular humans, against N. meningitidis of any serogroup, in particular against serogroup B. This immune response includes in particular, a bactericidal immune response wherein bactericidal antibodies are induced against N. meningitidis. By “bactericidal antibody” is meant antibodies able to kill the bacteria in the presence of complement (which is a component of the humoral immune system of mammals). The antibodies produced as part of the immune response upon administration of the immunogenic composition may be identified as “bactericidal antibodies” in a serum bactericidal assay using an appropriate source of complement, according to methods known in the art.

### Coverage of Protection

N. meningitidis species is genetically and antigenically highly diverse. Multilocus sequence typing (MLST) was first developed in the late 1990s for the meningococcus. It is a highly reliable and reproducible characterization method, which assesses variation at multiple genetic loci using nucleotide sequencing. More than 6751 sequence types (STs) have been assigned for N. meningitidis strains. While meningococcal diversity is extensive, it is highly structured. Studies of variation at housekeeping loci, initially by multilocus enzyme electrophoresis and more recently by MLST, had identified 37 groups of closely related meningococci at the time of writing, accounting for 61% of the meningococcal isolates represented in the PubMLST database. These groups, known as clonal complexes, have become the predominant unit of analysis in meningococcal population biology and epidemiology. A minority of clonal complexes, the so-called hyper-invasive lineages, are responsible for a disproportionate number of cases of disease worldwide and can be over-represented in collections of isolates from diseased patients by as much as two orders of magnitude, relative to their prevalence in asymptomatic carriage (see Table 2 herein after).

TABLE 2
Characteristics of the most important clonal complexes of Neisseria meningitidis (data compiled from the PubMLST database Jun. 2, 2009).

| | | | | | | | Disease/ | |
|---|---|---|---|---|---|---|---|---|
| | MLEE | No. | No. | Dominant serogroups | Dominant | Dominant | carriage | |
| ST-complex | designation | isolates | STs | (%) | PorA | FetA | ratio | Main origin |
| ST-1 complex | Subgroup I/II | 204 | 49 | A (97) | 5-2, 10 | F3-5 | 5.5 | Russia, China |
| ST-5 complex | Subgroup III | 627 | 33 | A (99) | 20, 9 | F3-1 | 19.5 | Africa |
| ST-8 complex | Cluster A4 | 283 | 107 | B (51), C (35) | 5-1, 2-2 | F3-6 | 24.5 | Europe |
| ST-11 complex | ET-37 complex | 1142 | 239 | C (57), W135 (24), B (12) | 5, 2 | F3-6 | 6.6 | Worldwide |
| ST-18 complex | Cluster J1 | 208 | 175 | B (85) | 22, 14 | F3-6 | 5.5 | Czech Republic, Poland |
| ST-22 complex | | 363 | 243 | W135 (52), NG (25) | 18-1, 3 | F4-1 | 0.6 | UK |
| ST-23 complex | Cluster A3 | 385 | 154 | Y (62), NG (18) | 5-1, 2-2 | F4-1 | 0.8 | Worldwide |
| ST-32 complex | ET-5 complex | 1028 | 350 | B (85) | 19, 15 | F5-1 | 3.5 | Worldwide |
| ST-35 complex | | 329 | 214 | B (59), NG (25) | 22-1, 14 | F4-1 | 0.5 | Worldwide |
| ST-41/44 complex | Lineage 3 | 1796 | 1274 | B (70) | 7-2, 4 | F1-5 | 1.2 | Worldwide |
| ST-53 complex | | 272 | 93 | NG (76) | 7-2, 30 | F1-7 | <0.1 | UK |
| ST-60 complex | | 225 | 148 | B (30), 29E (22), NG (19) | 5, 2 | F1-7 | 0.7 | Europe |
| ST-103 complex | | 127 | 84 | B (26), NG (22), C (16) | 18-1, 3 | F3-9 | 1.2 | Worldwide (-Africa) |
| ST-162 complex | | 140 | 63 | B (74), NG (13) | 22, 14 | F5-9 | 0.8 | Worldwide |
| ST-167 complex | | 201 | 144 | Y (47), NG (36) | 5-1, 10-4 | F3-4 | 0.5 | Worldwide |
| ST-198 complex | | 166 | 76 | NG (76) | 18, 25-15 | F5-5 | <0.1 | Worldwide |
| ST-213 complex | | 187 | 165 | B (74), NG (16) | 22, 14 | F5-5 | 0.6 | UK |
| ST-254 complex | | 148 | 107 | NG (35), B (24), 29E (12) | 5-1, 16 | F1-7, F3-6 | 0.5 | Worldwide |
| ST-269 complex | | 415 | 312 | B (73) | 22, 9 | F5-1 | 2.8 | Worldwide |
| ST-334 complex | | 106 | 64 | C (58), B (33) | 5-1, 2-2 | F1-5 | 5.7 | UK |

As shown in the above table, strains of e.g., serogroup B, belong to several clonal complexes. In particular, serogroup B strains are highly represented among significant invasive clonal complexes, including major clonal complexes spread world-wide i.e., ST-8, ST-18, ST-32, ST-41/44, ST-162 and ST-269 clonal complexes, as well as clonal complex ST-11, remarkable for its very low rate of carriage relative to high incidence of disease.

It is therefore highly desirable to evaluate the protection coverage provided by a polypeptide of the invention. To determine whether they are likely to give broad coverage across strains of serogroup B, representative strains of that serogroup among major clonal complexes (6 ST complexes or groups) were selected and effectiveness in term of protection coverage of these various protein antigens and combinations thereof was tested against these strains.

This protection coverage may be evaluated by serum bactericidal activity (SBA) assay which reflects the ability of a given antigen to elicit bactericidal antibodies. The SBA assay measures functional activity of antibody through complement-mediated antibody lysis of the bacteria. Serum bactericidal activity has been accepted as a valid surrogate for predicting the clinical efficacy of serogroup B meningococcal vaccines.

Indirect evidence of SBA assay providing surrogate of protection came from studies by Goldschneider and colleagues in 1969 where an inverse correlation between the incidence of disease and the prevalence of serum bactericidal activity in human serum SBA against MenA, MenB and MenC were reported. In their prospective study, the SBA titer in serum was measured using human complement (e.g., endogenous complement or exogenous serum from a healthy adult who lacked intrinsic bactericidal activity). They demonstrated in incoming recruits to a US Army base that the presence of serum bactericidal activity strongly indicated resistance to meningococcal disease. This led to the establishment of SBA assay as the immunological surrogate of protection against meningococcal disease.

Indeed, in clinical trials of MenB vaccines, the measurement of the increase of the SBA titer after vaccination compared to the SBA basal titer (before any administration of the meningococcal vaccine) is an established clinical end point. Seroconversion is considered to be met when an SBA titer is superior or equal to 4. This approach was validated in 2005 at a World Health Organization sponsored meningococcal serology standardisation workshop and is based upon evidence from a number of efficacy studies of OMV vaccines.

Animal SBA assays achieved in upstream research are as well commonly acknowledged as a surrogate of protection for meningitidis vaccines. In the context of the present invention, initial SBA assays were first carried out against the homologous strain (‘homologous’ assays). Then, antigens, in particular those that gave positive results in the homologous SBA assay were taken forward into ‘heterologous’ SBA assays, in which the antigens-specific corresponding sera were tested against different strains, to give an indication of the effectiveness of strain coverage which may be obtained. The inventors of the present invention have determined that when the SBA titer (fold-increase compared to a negative control group) is superior or equal to 16 in homologous SBA assay, or superior or equal to 8 in heterologous SBA, protection is considered to be met.

An optimal vaccine shall give a broad coverage against a panel of representative strains of relevant invasive clonal complexes.

Accordingly, an immunogenic composition according to the invention is particularly useful for inducing an immune response i.a., a bactericidal immune response, against N. meningitidis strains of (i) the clonal complexes of the hyper-invasive lineage (invasive clonal complexes); (ii) the clonal complexes wherein strains of serogroup B are prevalent (highly represented), those complexes being or not prevalent worldwide, advantageously prevalent worldwide; and/or (iii) clonal complexes ST8, ST11, ST18, ST32, ST41/44, ST162, and/or ST269. The immunogenic composition is more particularly useful against N. meningitidis strains of serogroup B belonging to clonal complexes, such as the ST11, ST18, ST32 and/or ST41/44 complex(es). The immunogenic composition may be characterized by strain coverage of at least 50%. In other words, it may induce a bactericidal immune response against at least 50%, 60%, 70%, 75%, 80%, 85%, 90%, 95% or 100% of N. meningitidis strains in one of the clonal complexes specified above, in particular the ST8, ST11, ST18, ST32, ST41/44, ST162 and/or ST269 complex(es).

In one embodiment, an immunogenic composition comprising a polypeptide comprising or consisting of a AusI fragment, is particularly useful against N. meningitidis strains of clonal complex ST269 e.g., of serogroup B.

Strain coverage may be determined as described in the experimental part of the specification, involving in particular (i) the selection of a collection of strains representative of the most important clonal complexes e.g. including ST8, ST11, ST18, ST32, ST41/44 and/or ST269 complex(es) and (i) the achievement of an SBA assay against each of the strains of the collection, such as described in the experimental part. Briefly, the whole test consist in administering the composition to a mammal, one or several times at appropriate interval; collecting the sera that may optionally be pooled (within a group of mammals submitted to identical administration); culturing the strains of the collection; and testing the individual sera or pooled serum and/or dilutions thereof against each strain in an SBA assay, such as the one described in the experimental part. The percentage of coverage is determined on the basis of the number of strains responding positively—that is, against which the bactericidal titer of e.g., the pooled serum, meets (e.g., equals or is superior to) the threshold value considered as indicative of a positive surrogate of protection—over the total number of strains tested. Alternatively, the bactericidal titer of individual sera within a group of mammals as defined above, may be determined and the geometric mean titer (GMT) established. In that case, the strain is considered to respond positively when the GMT meets (e.g., equals or is superior to) the threshold value considered as indicative of a positive surrogate of protection.

An immunogenic composition according to the invention may be used as a pharmaceutical composition, in a prophylactic or therapeutic manner. Typically, it may be used as a vaccine composition for protecting against N. meningitidis infections e.g., for treating or preventing N. meningitidis infections. N. meningitidis induces a large range of infections from asymptomatic carriage to invasive diseases e.g., meningitidis and/or septicemia. Typically, the immunogenic or pharmaceutical composition of the invention comprises a therapeutically or prophylactically effective amount of a polypeptide of the invention. A therapeutically and/or prophylactically effective amount of a polypeptide of the invention may vary according to factors such as the disease state, age, sex, and weight of the individual, and the ability of the polypeptide of the invention, to elicit a desired therapeutic and/or prophylactic result.

The composition according to the invention may be administered as a dose wherein the amount of a polypeptide of the invention depends on various conditions including e.g., the weight, the age and the immune status of the recipient. As a matter of guidance, it is indicated that a dose of the composition of the invention may comprise a therapeutically and/or prophylactically effective amount of a polypeptide of the invention, which may be from 10 μg to 1 mg, e.g. about 50 μg.

‘Prevention’ refers to prophylactic treatment, wherein a composition of the invention is administered to an individual with no symptoms of meningitis and/or septicemia and/or no detectable N. meningitidis infection. Said prophylactic treatment is preferably administered with the aim of preventing or reducing future N. meningitidis infection.

Within the meaning of the invention, the terms “composition for preventing or for prevention” intend to means, with reference to an N. meningitidis infection, a reduction of risk of occurrence of said infection and/or symptoms associated with said infection.

‘Treatment’ includes both therapeutic treatment and prophylactic or preventative treatment, wherein the object is to prevent or slow down the infection or symptoms of disease. Those in need of treatment include those already with the disorder as well as those prone to have the disorder or those in whom the disorder is to be prevented. The terms ‘therapy’, ‘therapeutic’, ‘treatment’ or ‘treating’ include reducing, alleviating or inhibiting or eliminating the symptoms or progress of a disease, as well as treatment intended to reduce, alleviate, inhibit or eliminate said symptoms or progress.

A further object of the invention is to provide a method of inducing an immune response, in particular a bactericidal immune response, against N. meningitidis, in particular against N. meningitidis of serogroup B, which comprises administering to an individual in need an immunogenic composition according to the invention. Still within the scope of the invention, it is provided a method of treating or preventing a N. meningitidis infection, in particular an infection of N. meningitidis of serogroup B, which comprises administering to a patient in need a composition according to the invention.

In order to achieve the desirable effect, the composition of the invention may be administered as a primary dose, in a primary immunisation schedule, one or several times, e.g., two or three times, at appropriate intervals defined in terms of week or advantageously, month. In a particular embodiment, the interval between the primary doses may be not less than one or two months, depending on the conditions of the subject receiving the doses. If needed, the primary doses may possibly be followed by a booster dose of the composition of the invention, which may be administered e.g., from at least 6 months, preferably at least one year to two-five years, after the last primary dose.

The composition according to the invention may be administered by any conventional routes in use in the vaccine field e.g. by parenteral route such as the sub-cutaneous or intramuscular route. In a particular embodiment, the composition is suitable for injection and formulated accordingly. It may be in a liquid form or in a solid form that, before administration, may be extemporaneously suspended in a pharmaceutically-acceptable diluent.

Also provided is a polypeptide of the invention in the manufacture of a medicament for the preventive or therapeutic treatment of a N. meningitidis infection, e.g. an infection of N. meningitidis of serogroup B, such as meningitis.

Also provided is a polypeptide or composition of the invention for use in a method of inducing an immune response to N. meningitidis, in particular N. meningitidis of serogroup B. Also provided is a polypeptide or composition of the invention for use in a method of preventing or treating a N. meningitidis infection, e.g. an infection of N. meningitidis of serogroup B, such as meningitis. In some embodiments, said method comprises administering said polypeptide or composition to a subject, in particular a subject in need thereof. According to one embodiment, a method of the invention may comprise the step of observing a preventing and/or a treating effect with regard to a N. meningitidis infection.

Also provided is a method of inducing an immune response to N. meningitidis, in particular N. meningitidis of serogroup B, which comprises administering a polypeptide or composition of the invention to an individual in need thereof. Also provided is a method of preventing or treating of meningitis, in particular N. meningitidis infection, e.g. an infection of N. meningitidis of serogroup B, such as meningitis, which comprises administering a polypeptide or composition of the invention to an individual in need thereof.

The invention will be further illustrated by the following figures, sequences and experimental part:

### BRIEF DESCRIPTION OF THE FIGURES

FIG. 1 shows the amino acid (SEQ ID NO: 1) and nucleic acid (SEQ ID NO: 2) sequences of IgA1 protease from N. meningitidis B strain MC58.

FIG. 2 shows the amino acid sequence of App from N. meningitidis B strain MC58 (SEQ ID NO: 3).

FIG. 3 shows the coding nucleic acid sequence of App from N. meningitidis B strain MC58 (SEQ ID NO: 4).

FIG. 4 shows the amino acid sequence of AusI from N. meningitidis B strain MC58 (SEQ ID NO: 5).

FIG. 5 shows the coding nucleic acid sequence of AusI from N. meningitidis B strain MC58 (SEQ ID NO: 6).

FIG. 6 is a graph showing cross-SBA and FACS results generated with construct SP503 in experiments A & B. SBA results are expressed in term of fold-increase. SE=surface exposure of IgA1P as assessed by FACS.

FIG. 7 is a graph showing cross-SBA results generated with constructs SP503, SP528 and SP530. SBA results are expressed in term of fold-increase.

FIG. 8 is a graph showing cross-SBA generated with construct SP531.

FIG. 9 is a graph showing cross-SBA generated with construct SP532.

FIG. 10 is a graph showing cross-SBA generated with construct SP533.

FIG. 11 is a graph showing cross-SBA and FACS results generated with constructs SP503, SP548 and SP550. SBA results are expressed in term of fold-increase. SE=surface exposure of IgA1P as assessed by FACS.

FIG. 12 is a graph showing cross-SBA results generated with constructs SP534, SP535, SP536 and SP537. SBA results are expressed in term of fold-increase.

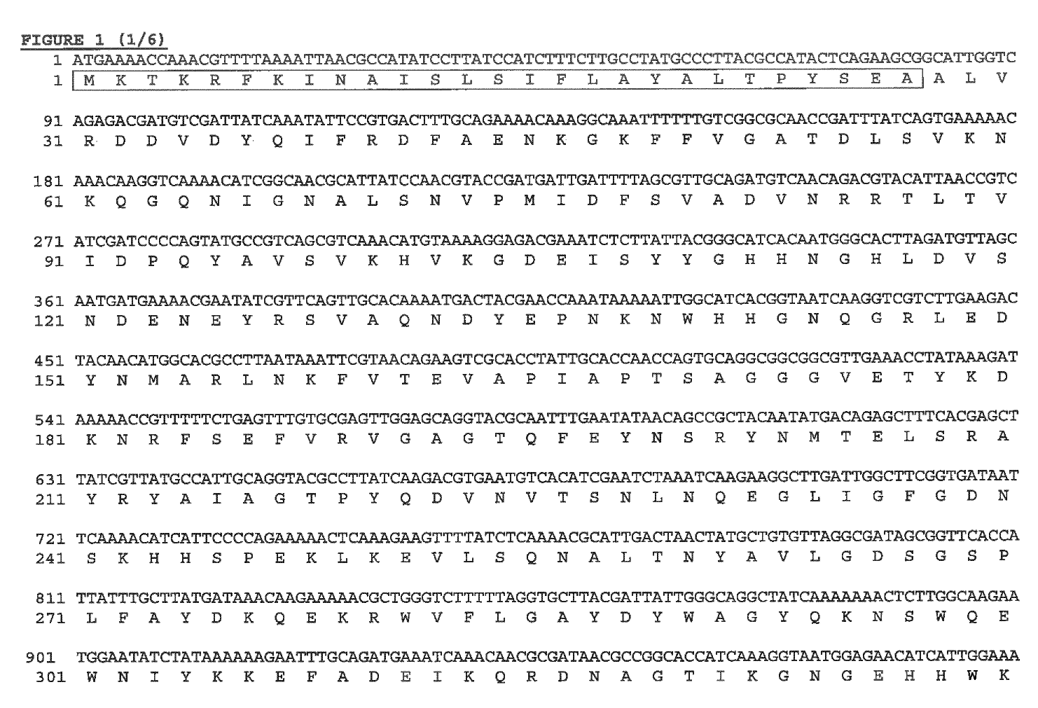

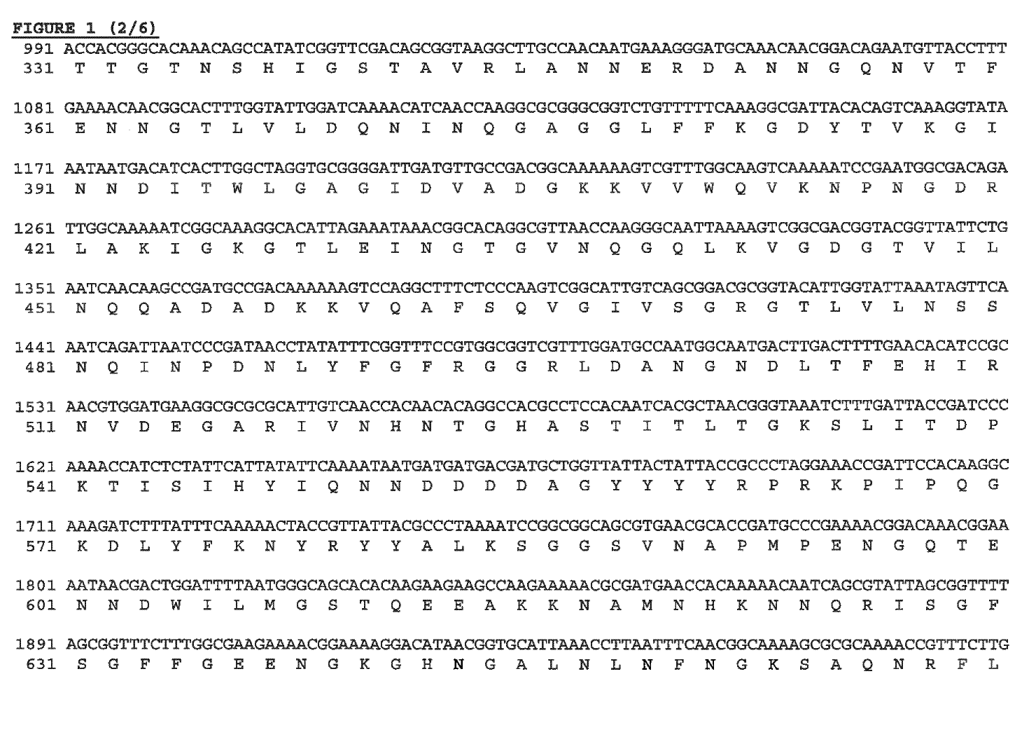

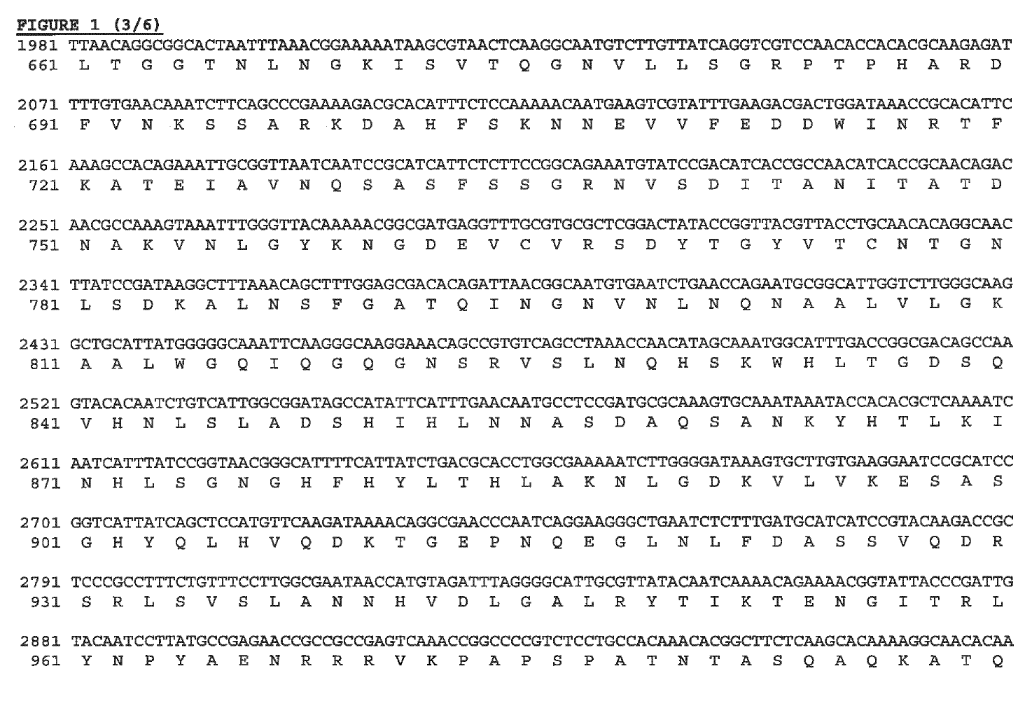

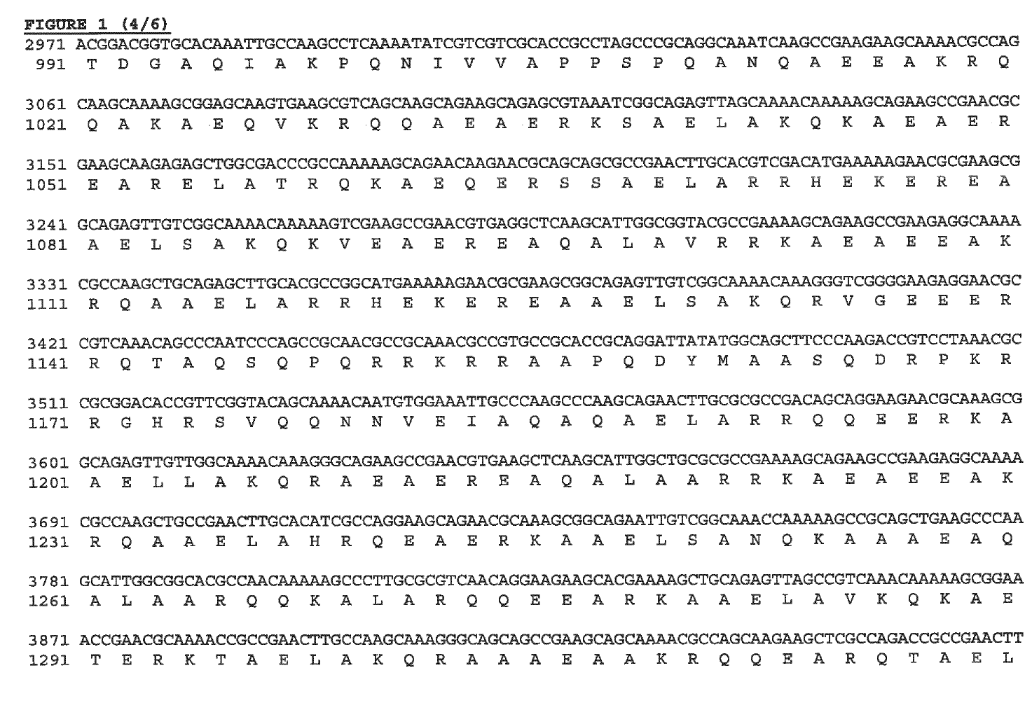

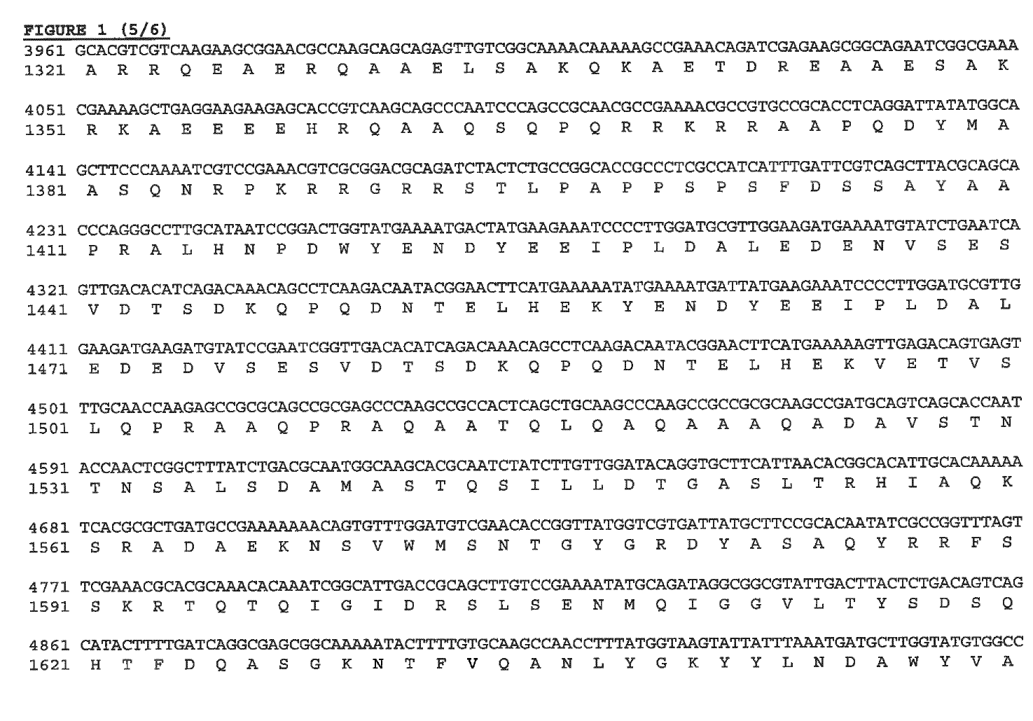

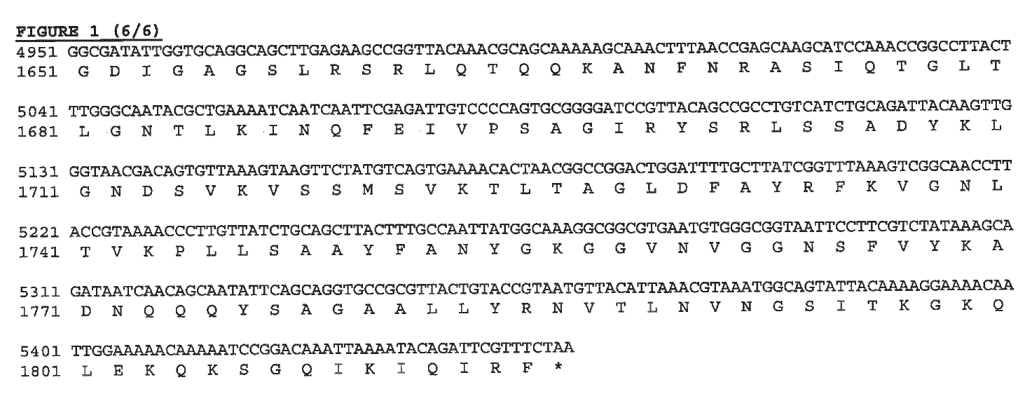

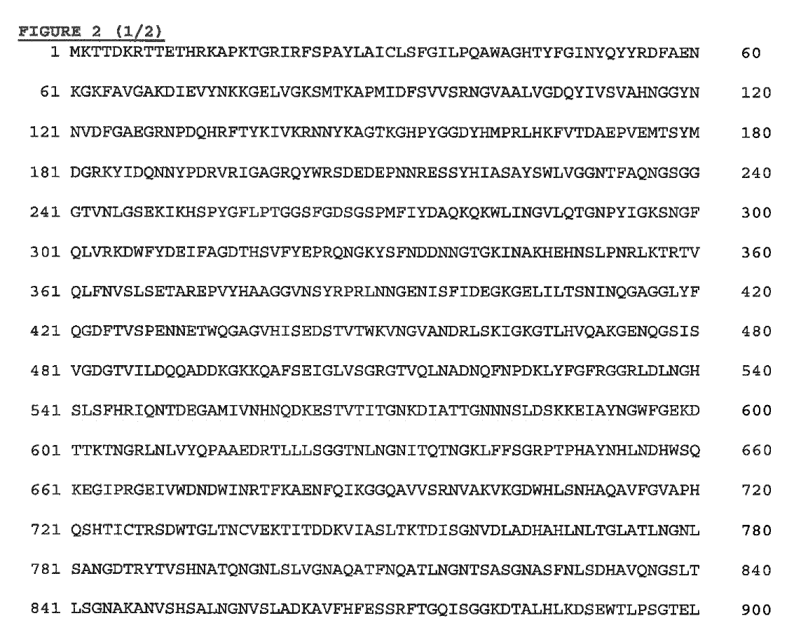

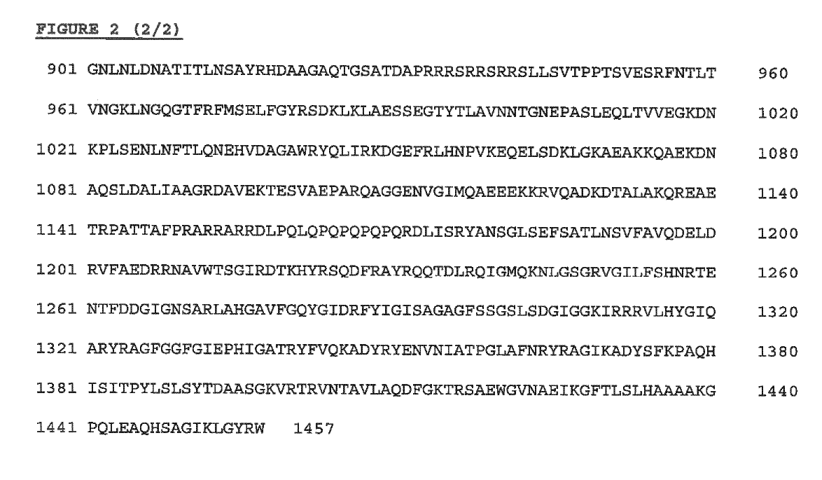

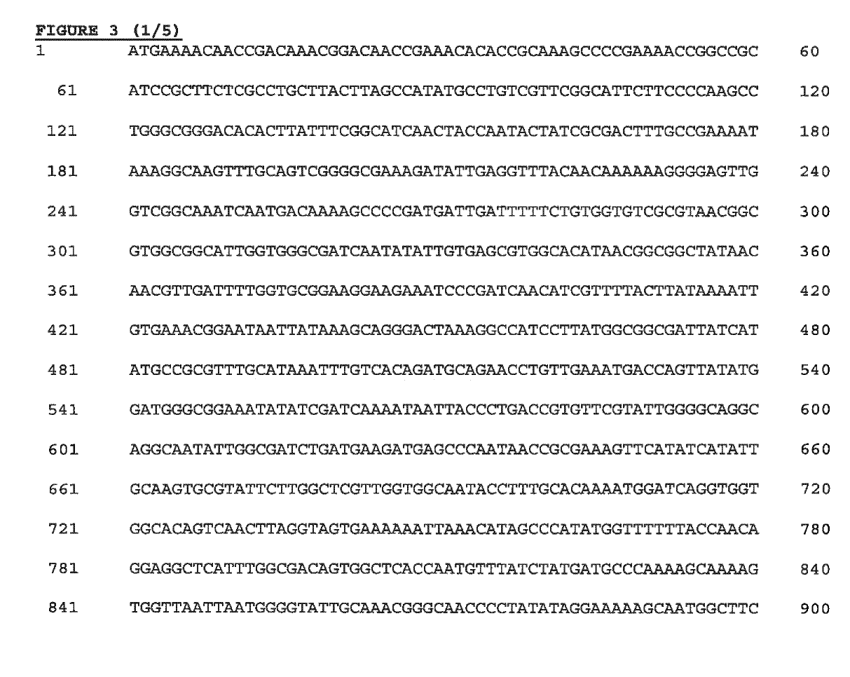

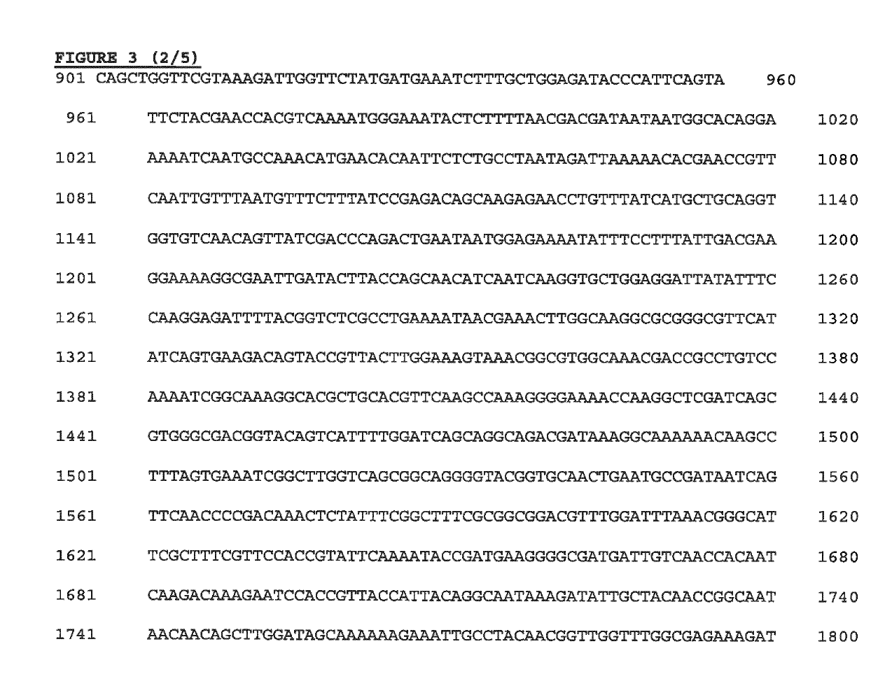

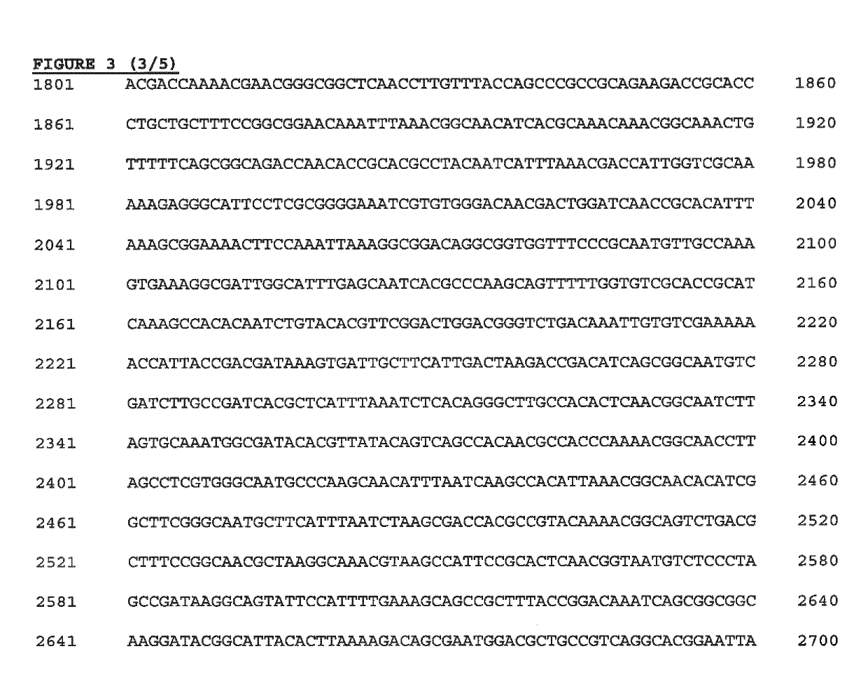

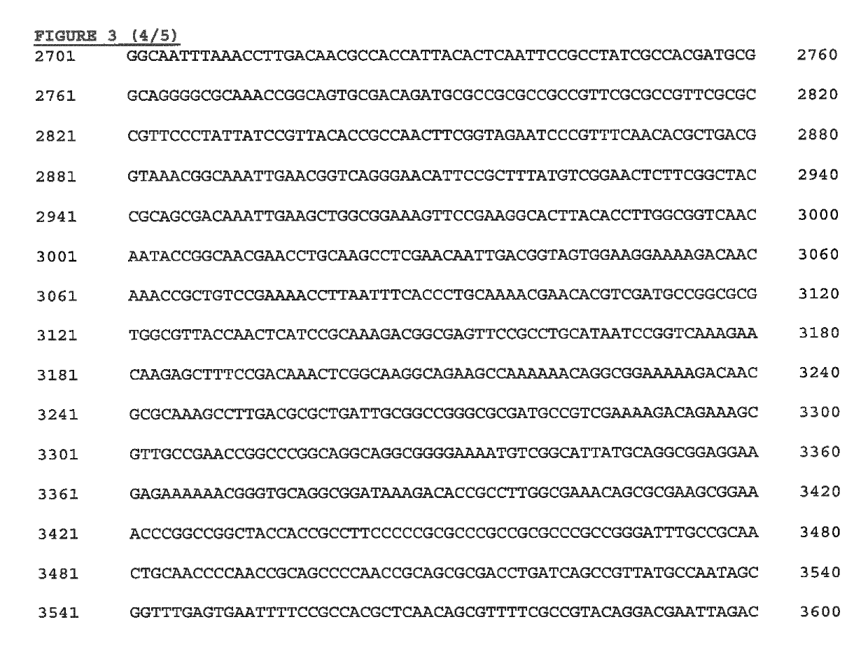

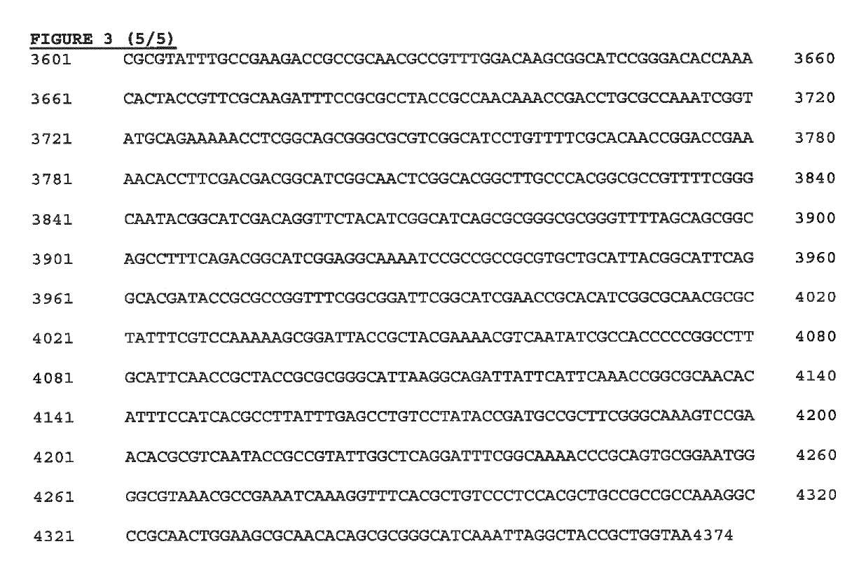

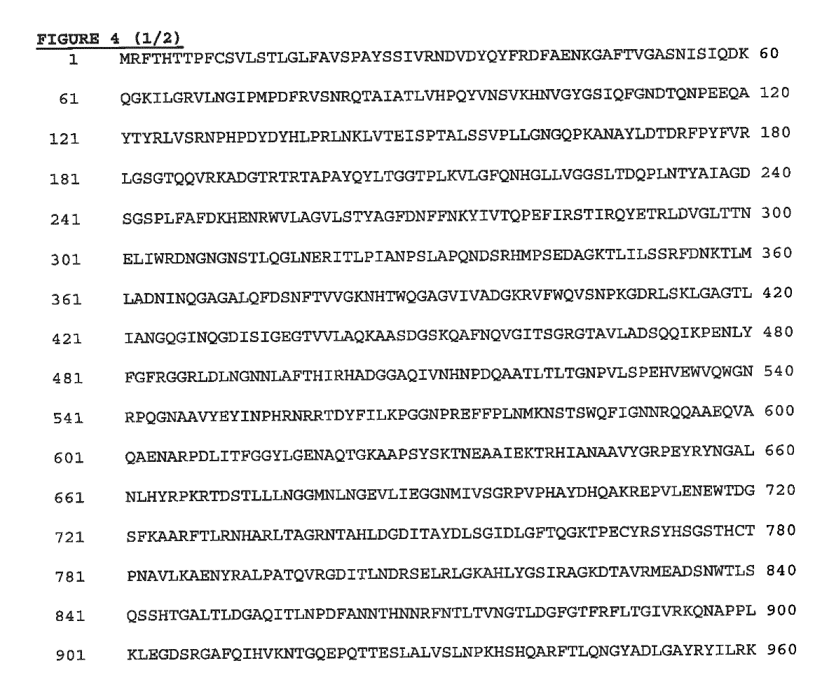

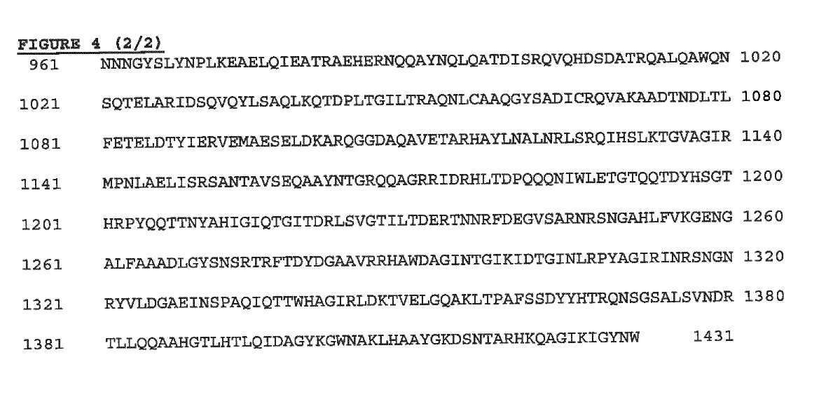

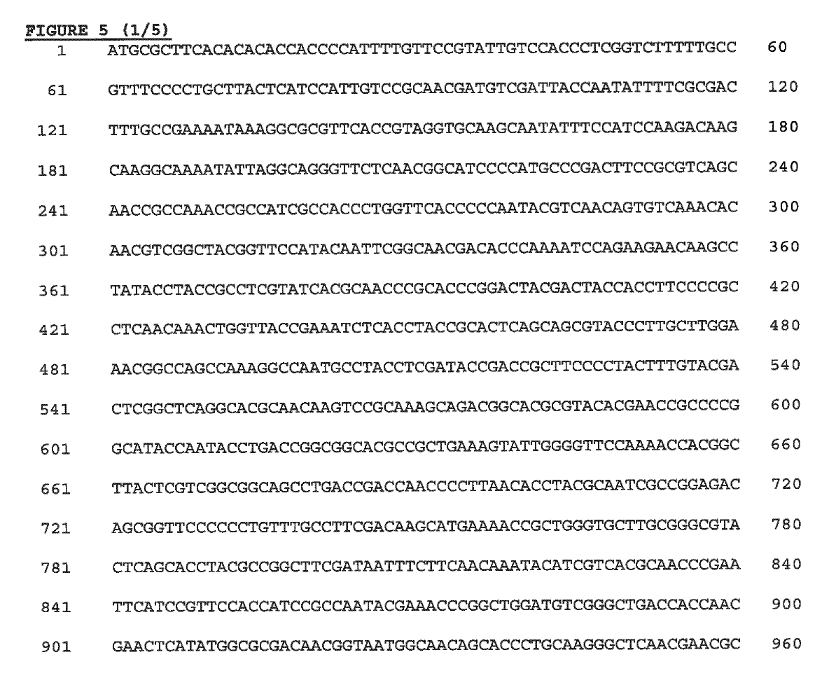

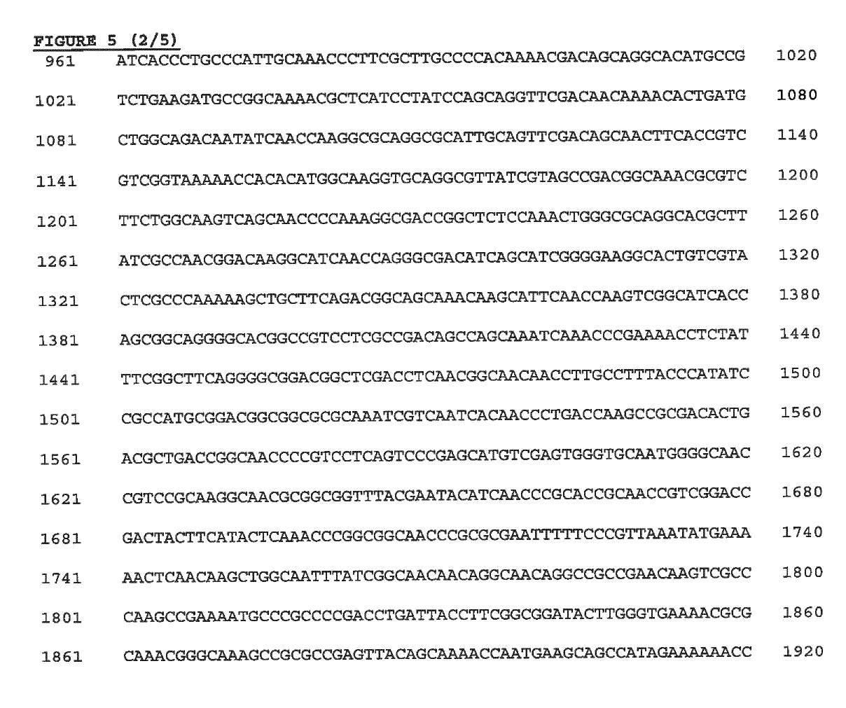

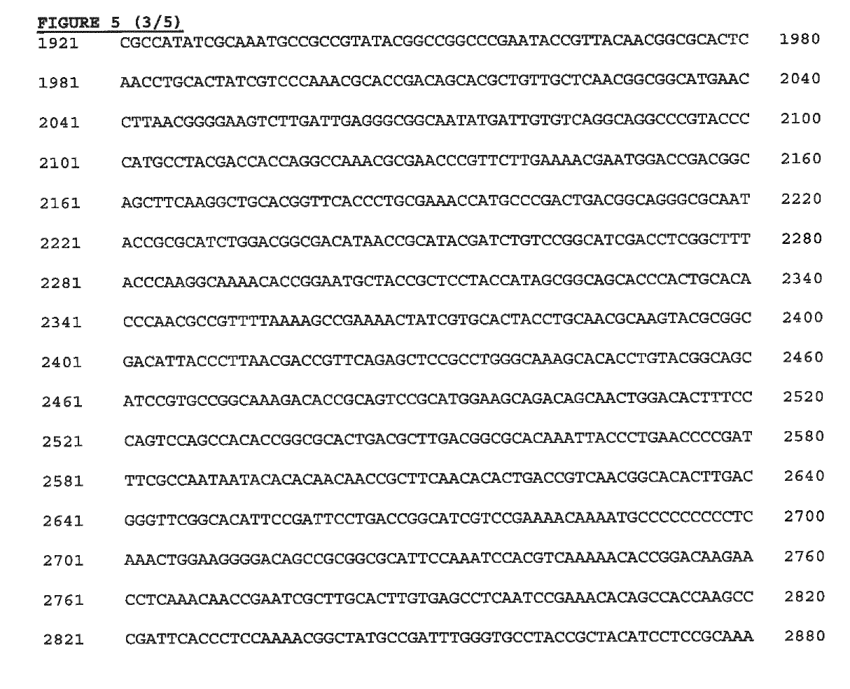

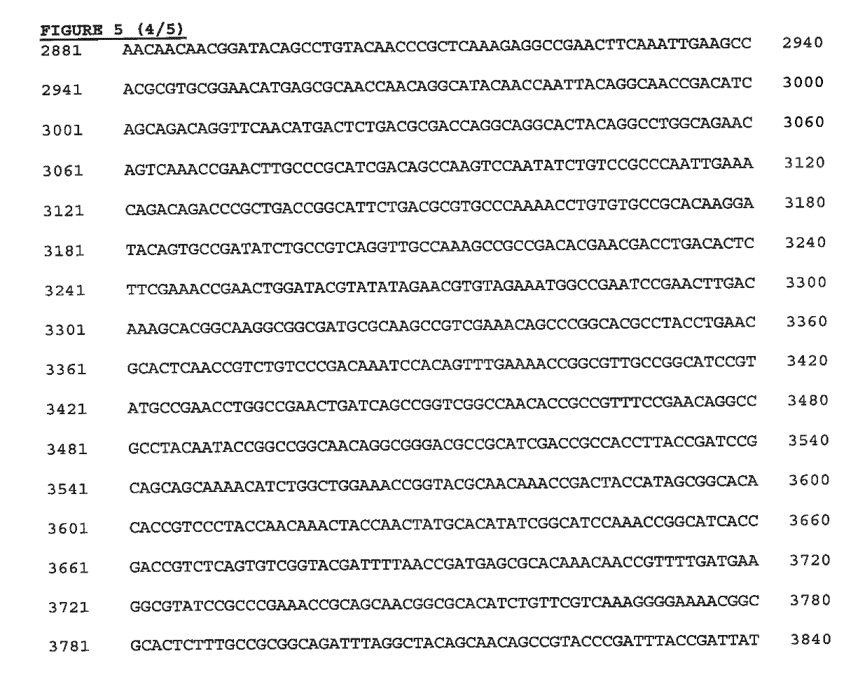

### BRIEF DESCRIPTION OF THE SEQUENCE LISTING

### SEQ ID NO:

1 Amino acid sequence of IgA1 protease from N. meningitidis B strain MC58
2 Coding nucleic acid sequence of IgA1 protease from N. meningitidis B strain MC58
3 Amino acid sequence of App from N. meningitidis B strain MC58
4 Coding nucleic acid sequence of App from N. meningitidis B strain MC58
5 Amino acid sequence of AusI from N. meningitidis B strain MC58
6 Coding nucleic acid sequence of AusI from N. meningitidis B strain MC58

### EXPERIMENTAL

### A—Material & Methods

### Constructs

The sequence information with respect to the iga, app and ausI genes of the N. meningitidis MC58 genome (respectively NMB0700, NMB1985 and NMB1998) was retrieved from the Entrez Gene database of the NCBI (National Center for the Biotechnology Information) at http://www.ncbi.nlm.nih.gov under the accession number NC_003112. This sequence information is particularly useful for designing the primers.

The genomic DNA of strain MC58 was purified using a purification kit (Roche). In order to generate constructs, ORFs (open reading frame) were amplified by PCR from the purified genomic DNA using appropriate primers. 5′ or 3′ primers were designed so that a His-tag may be introduced (and when appropriate a spacer between the His-tag and the N-ter amino acid of the protein) as well as restriction sites. Then the PCR products were first cloned in an intermediate cloning vector and then transferred into the expression plasmid pET-cer which is a pET-28 plasmid (Novagen) stabilized by insertion of a stabilizing element (cer fragment).

A large number of IgA1P constructs were produced: SP502, SP503, SP528, SP530, SP531, SP532, SP533, SP548, SP550. They are more particularly described as follows:

SP502 and SP503 both start with Alanine 28 and ends with Alanine 1584 (amino acid numbering is based on the complete IgA1P amino acid sequence NMB0700). A His-tag is added at the N-ter end, separated from Ala 28 by a spacer constituted with four glycines and one serine (N-ter to C-ter). SP503 further comprises the Ser 267 Val mutation.

SP528 and SP548 both start with Alanine 28 and end with Alanine 1005 (amino acid numbering is based on the complete IgA1P amino acid sequence NMB0700) and comprise the Ser 267 Val mutation. In SP528, a His-tag is added at the N-ter end, separated from Ala 28 by a spacer constituted with four glycines and one serine (N-ter to C-ter). In SP548, a His-tag is added at the C-ter end, without spacer.

SP530 and SP550 both consist from N-ter to C-ter in (i) the IgA1P sequence exhibiting the Ser 267 Val mutation, starting with Alanine 28 and ending with Glutamic acid 966 (amino acid numbering is based on the complete IgA1P amino acid sequence NMB0700) fused to (ii) the App sequence starting with Glutamine 1061 and ending with Alanine 1187 (amino acid numbering is based on the complete App amino acid sequence NMB 1985). In SP530, a His-tag is added at the N-ter end, separated from Ala 28 by a spacer constituted with four glycines and one serine (N-ter to C-ter). In SP550, a His-tag is added at the C-ter end, without spacer.

SP532 consist from N-ter to C-ter in (i) the IgA1P sequence exhibiting the Ser 267 Val mutation, starting with Alanine 28 and ending with Glutamic acid 966 (amino acid numbering is based on the complete IgA1P amino acid sequence NMB0700) fused to (ii) the App sequence starting with Glutamine 1061 and ending with Serine 1224 (amino acid numbering is based on the complete App amino acid sequence NMB1985). A His-tag is added at the N-ter end, separated from Ala 28 by a spacer constituted with four glycines and one serine (N-ter to C-ter).

SP531 and SP533 consist from N-ter to C-ter in (i) the IgA1P sequence exhibiting the Ser 267 Val mutation, starting with Alanine 28 and ending with Glutamic acid 966 (amino acid numbering is based on the complete IgA1P amino acid sequence NMB0700) fused to (ii) the AusI sequence starting with Alanine 974 and ending with Alanine 1161 (SP531) or Serine 1198 (amino acid numbering is based on the complete AusI amino acid sequence NMB1998). A His-tag is added at the N-ter end, separated from Ala 28 by a spacer constituted with four glycines and one serine (N-ter to C-ter).

App and AusI constructs were also produced: respectively (i) SP534, SP535, and (ii) SP536, SP537. They are described as follows:

SP534 and SP535 both start with Glycine 43 and end with Serine 1224 (amino acid numbering is based on the complete App amino acid sequence NMB1985). A His-tag is added at the N-ter end, separated from Ala 28 by a spacer constituted with four glycines and one serine (N-ter to C-ter). SP535 further comprises the Ser 267 Val mutation.

SP536 and SP537 both start with Serine 26 and end with Serine 1198 (amino acid numbering is based on the complete AusI amino acid sequence NMB1998). A His-tag is added at the N-ter end, separated from Serine 26 by a spacer constituted with four glycines and one serine (N-ter to C-ter). SP537 further comprises the Ser 241 Val mutation.

As a matter of additional guidance, the construction of the expression plasmids for SP502 and SP503 is further described as follows:

First the ORF is amplified by PCR from the purified MC58 genomic DNA, using appropriate primers and the Platinum® Pfx Polymerase (Invitrogen) according to the protocol of the supplier. Then the PCR product is cloned into the intermediate vector: PCR Blunt TOPO vector (PCR®-TOPO®-BluntII) according to the protocol of the supplier. The ligation product is transformed into competent cells TOP10 bacteria supplied with the kit (Invitrogen). The selection of recombinant clones is performed on LB+kanamycin. The plasmid is checked by enzymatic digestion and verification of the restriction profile. The sequencing of the insert validates the plasmid.

Then the ORF is extracted from the intermediate vector by appropriate enzymatic digestion (double digestion enzyme NcoI+BamHI restriction sites) using the originally inserted restriction sites for transfer into an expression plasmid. The extracted fragment of interest is isolated by agarose gel migration and cutting the corresponding bands and then purified by electro-elution. Plasmid pET-cer is prepared using the same protocol. The extracted fragment of interest is then assembled with the pET-cer using T4 DNA ligase (Invitrogen) according to the protocol of the supplier to give the expression plasmid pSP502. The ligation product is transformed into competent TOP10 bacteria. The selection of recombinant clones is performed on LB+kanamycin. The resulting plasmid pSP502 is checked by enzymatic digestion and verification of the restriction profile. The sequencing of the insert validates the plasmid in which the ORF is placed.

In order to produce the plasmid able to express the ORF encoding SP503 a further mutagenesis step to suppress the active catalytic site is achieved as follows:

Overlap extension PCR using the pSP502 is performed to amplify the ORF in two overlapping PCR fragments. The overlapping central primers were designed to introduce the mutation. A third reaction was then used to assemble the first two fragments into one. The reactions are performed with Platinum® Taq DNA Polymerase High Fidelity (Invitrogen) according to the protocol of the supplier. The two overlapping central primers also insert an original restriction site to facilitate the selection of clones carrying the mutation. The mutated ORF is selected using the restriction site created during the mutagenesis; and substituted for the corresponding non-mutated ORF into pSP502 to give SP503.

The nucleotide sequences encoding the IgA1P constructs SP528 and SP548; the App constructs SP534 and SP535; the AusI constructs SP536 and SP537; as well as the IgA1P fusion constructs SP530, SP531, SP532, SP533 and SP550; were conceived and designed by bioinformatics using the software pack Vector NTI (Invitrogen) and accordingly, chemically synthetized de novo (Geneart). The synthetized sequences were cloned in an intermediate plasmid of pUC type; then transferred into the pET-cer plasmid to be placed under the control of the T7 promoter (from pET-28).

Transformation into the Expression Strain

The expression plasmids were transformed into the expression E. coli strain BL21 (DE3) (Novagen) according to the protocol of the supplier. The selection of recombinant clones was performed on LB+kanamycin.

### Protein Expression and Purification

### Cell Culture

BL21 (DE3) E. coli strains transformed by one of the plasmids pSP502, pSP503, pSP528, pSP530, pSP531, pSP532, pSP533, pSP548, pSP550, pSP534, pSP535, pSP536 and pSP537 were seeded at a ratio 1:500 in Luria Bertani broth (LB) medium supplemented with kanamycin 30 μg/ml and at 37° C. under stirring (220 rpm) up to a O.D.600 nm of from 0.6 to 0.8. The IPTG is added at 1 mM final and the induction is pursued at 37° C. for 3 hrs. Bacterial cells are harvested by centrifugation and pellets stored at −20° C.

### Purification of Recombinant Proteins

Upon thawing, bacteria were washed in PBS and centrifuged. Pellet (P0) was resuspended in a buffer (PBS or Tris-HCl pH 8) containing lysozyme 100 μg/ml, MgCl2 1 mM and Triton X-100 0.1%; and incubated 15 min at 4° C. The suspension was viscous, free of visible aggregates and slightly translucent. Benzonase (1 U/ml final) was then added and the mixture was sonicated. The viscosity of the suspension must have disappeared. The insoluble proteins were pelleted after centrifugation. The pellet (P1) was resuspended in a buffer (PBS or Tris-HCl, pH 8) containing Triton X-100 0.1% and Urea 2 M. After centrifugation, the pellet (P2) was resuspended in a buffer (Tris-HCl 50 mM, pH 9) containing NaCl 300 mM, Urea 8 M and, optionally Triton X-100 0.1%. Upon centrifugation, the supernatant containing the His-tag protein was recovered and diluted to Urea 4 M while adding Tris-HCl 50 mM pH 9, NaCl 300 mM and zwittergent 3.14 (1% final).

The diluted supernatant was further purified by nickel chelation chromatography on an IMAC column using an imidazole elution gradient (0 to 250 mM). Elution fractions containing the protein were pooled and extensively dialysed against a buffer (Tris-HCl 20 mM, NaCl 150 mM or PBS pH 8) containing Urea 4 M to remove imidazole. Refolding was achieved by further dialysis against a buffer (Tris-HCl 20 mM, NaCl 150 mM or PBS pH 8) containing arginine 0.5 M.

In what follows, the purification of MC58 IgA1 protease SP503, SP548 and SP550 is more particularly described as an additional matter of example.

### Preparation of MC58 IgA1P, SP503, SP548 and SP550 Extracts for Purification on an IMAC Column.

The bacterial pellets corresponding to 500 ml of culture are gently washed in PBS and bacterial suspensions are centrifuged. Pellets (P0) are resuspended in PBS pH 8 (SP503, SP550) or Tris-HCl 50 mM pH 8 (SP548); each buffer being complemented with lysosyme 100 μl/ml, MgCl2 1 mM, Triton X100 0.1%. Incubation is achieved at 4° C. 15 min under mild stirring.

Benzonase is added at about 1 unit/ml. Suspension are further incubated at 4° C. 15-30 min. For SP548, the suspensions are then gently sonicated 1 min in ice and stirred 20 min at 4° C.

Suspensions are centrifuged 20 min at 30 000 g, 4° C. Pellets (P1) are resuspended in PBS pH 8 (SP503, SP550) or Tris-HCl 50 mM pH 8 (SP548); each buffer being complemented with Triton X100 0.1% and urea 2 M. The suspensions are incubated for 1 hr at 4° C. under mild stirring and centrifuged 20 min at 30 000 g 4° C. The SP503, SP548 and SP550 pellets (P2) are resuspended in Tris-HCl 50 mM, NaCl 300 mM, Urea 8 M, pH 9.0 (complemented with Triton X-100 0.1% for SP548 and SP550). The suspensions are incubated at 4° C. overnight under mild stirring and then centrifuged 20-30 min at 30 000 g 4° C. Supernatants are recovered.

### Purification of MC58 IgA1P SP503, SP548 and SP550

The supernatant is diluted to a final concentration of Tris-HCl 50 mM, NaCl 300 mM, Urea 4 M, pH 9.0. Zwittergent 3.14 is added to 1% final.

An IMAC column (Chelating Sepharose Fast Flow from GE HealthCare) is prepared with 50 ml of a chelating gel charged with nickel (NiSO4 10% in water). The column is equilibrated with buffer A (Tris-HCl 50 mM, NaCl 300 mM, Urea 4 M, pH 9.0) at a flow rate of 2 ml/min. This flow rate is applied to the following purification steps.

About 100-150 ml of the SP503, SP548 or SP550 diluted supernatant to be purified are applied onto the equilibrated column.

About 3 column volumes of Buffer A are added. Then 3 column volumes of a gradient is applied to: 100 to 80% buffer A+0% to 20% buffer B (Buffer A+250 mM Imidazole). This is followed by (i) 3 column volumes of 80% buffer A+20% buffer B; and then (ii) 4 column volumes of buffer B.

The SP503 fractions eluted at 50 mM imidazole are pooled and dialysed overnight against PBS urea 4 M and stored at −80° C. After dialysis against 4 M urea, about 19 mg of SP503 are recovered (about 0.3 mg/ml). Before use, SP503 is renatured by extensive dialysis against Tris-HCl 20 mM, NaCl 150 mM, Arginine 0.5 M, pH 8.0. The final SP503 concentration is about 0.40 mg/ml.

The SP548 and SP550 fractions each elute at 250 mM imidazole. Fractions are pooled and dialysed overnight against Tris HCl 20 mM, NaCl 150 mM, urea 4 M, pH 8.0.

After dialysis against 4 M urea, about 50 mg of SP548 are recovered (about 3.40 mg/ml). The concentration is decreased to about 0.4 mg/mL. SP548 is renatured by extensive dialysis against Tris-HCl 20 mM, NaCl 150 mM, Arginine 0.5 M, pH 8.0 and stored at −80° C. (0.50 mg/ml).

After dialysis against 4 M urea, about 50 mg of SP550 are recovered (about 3.15 mg/ml). The concentration is decreased to about 0.7 mg/ml. SP550 is renatured by extensive dialysis against Tris-HCl 20 mM, NaCl 150 mM, Arginine 0.5 M, pH 8 and stored at −80° C. (0.75 mg/ml).

### Immunogenicity, Bactericidal Activity & Flow Cytometry Analysis

### Bacterial Strains and Growth Conditions

A set of 26 wild-type serogroup B N. meningitidis isolates that were isolated from geographically distinct locations at different date of isolation and that represented diverse MLST clonal complexes were selected for this study. They are listed in Table 3. The majority of the strains were kindly provided by Drs D. A Caugant (NIPH, Norway), D. Martin (EZR, New-Zealand), M. K Taha (IP, Paris), M. A. Diggle (SHLMPRL, Scotland), L. Saarinen (NPHI, Finland).

MenB strains were grown overnight at 37° C. with 10% CO₂ on Brain Heart Infusion (BHI) agar (Difco) plates. Then, the bacteria were harvested from plates and inoculated into BHI broth (Difco) alone or supplemented with or without 30 μM desferal which is a chelator of divalent cations. Cultures were analyzed after 2.5 hours that correspond to an early exponential growth phase.

### Production of Mice Antisera

To obtain specific immune sera, outbred CD1 mice were immunized 3 times on days 0, 21 and 35, by subcutaneous route, with 10 μg/mouse of the antigen of interest co-injected with adjuvant AF04 [oil-in-water emulsion as described in WO 07/006939, containing the Eisai product ER 804057 (also known as E6020, described in U.S. Pat. No. 7,683,200) as TLR4-agonist. AF04 is described in Examples 1 and 2 of WO 07/080308].

Blood samples were collected on day 42. Blood samples were collected in vacutainer vials containing a coagulation activator and a serum separator gel (BD, Meylan France). Tubes were centrifuged for 20 min at 2600 g in order to separate serum from cells. Sera were transferred into Nunc tubes and heat-inactivated for 30 min at 56° C. They were stored at −20° C. until the assays were performed.

### Serum Bactericidal Activity Assay

N. meningitidis strains were grown overnight at 37° C. with 10% CO₂ on BHI agar (Difco) plates. The bacteria were then harvested from the plates and inoculated into BHI broth (Difco) alone or supplemented with 30 μM desferal which is a chelator of divalent cations. The cultures were analyzed after 2.5 hours, which corresponds to early exponential growth phase. The bactericidal activity of specific mouse sera was evaluated using as complement source pooled baby rabbit serum as described earlier with slight modifications (Rokbi et al., Clin. Diagnostic Lab. (1997) 4 (5): 522). Briefly, 50 μl of two-fold serial dilutions of serum were added to 96-well microtiter plates (Nunc) and incubated with 25 μl of a meningococci suspension adjusted to 4×10³ CFU/ml and 25 μl of baby rabbit complement. After 1 hr of incubation at 37° C., 50 μl of the mixture from each well was plated onto MHA plates. The plates were incubated overnight at 37° C. in 10% CO₂. The bactericidal titer of each serum was expressed as the inverse of the last dilution of serum at which ≥50% killing was observed compared to the complement control.

The SBA assay is commonly acknowledged as a surrogate of protection for vaccines against N. meningitidis. When the SBA titer is superior or equal to 16 in homologous SBA assay, or superior or equal to 8 in heterologous SBA, protection is considered to be met.

### Flow Cytometry Analysis

The ability of polyclonal antisera, elicited by the recombinant proteins, to bind to the surface of live MenB strains was determined using a flow cytometric detection of indirect fluorescence assay. A culture sample was centrifuged and washed once with 1×PBS (Eurobio). The final pellet was resuspended in PBS with 1% of bovine albumin (BSA, Eurobio) at a density of 10⁸ CFU/ml. To 20 μl of bacteria, 20 μl of dilutions of pooled serum were added in 96 deep-well plate (Ritter). For each serum, 3 dilutions were tested on a range going from 1/5 to 1/1000. The plate was incubated for 1 h at 37° C. with shaking. The bacteria were centrifuged, washed once with PBS 1% BSA and resuspended with 100 μl of goat anti-mouse IgG (H and L chains) conjugated to FITC (Southern Biotech) diluted 100-fold. The plate was incubated for 30 minutes at 37° C. with shaking in the dark. The bacteria were washed twice with PBS 1% BSA and fixed with 0.3% formaldehyde in PBS buffer overnight at +4° C. in the dark. The bacteria were centrifuged, the formaldehyde solution was discarded and the bacteria were finally washed once and dissolved in PBS 1% BSA. The fluorescent staining of bacteria was analyzed on a Cytomics FC500 flow cytometer (Beckman Coulter). The fluorescent signal obtained for bacteria incubated with the polyclonal antisera specific for proteins injected with adjuvant was compared to the signal obtained for bacteria incubated with the antisera of mice injected with buffer+adjuvant.

Surface Exposure (SE) is expressed in terms of detection level ranging from [−] to [++++] depending on the highest dilution of the pooled antisera at which surface exposure is detected: [−] at a dilution <1/20e; [+] at a 1/20e dilution: [++] at a 1/200e dilution; [+++] at a 1/2000e dilution; and [++++] at a dilution >1/2000e.

### B—Results

All the constructs were administered to mice in the presence of adjuvant AF04. Polyclonal antisera thereof were individually assayed for serum bactericidal activity (SBA) against homologous strain or as a pool against a panel of heterologous strains. In addition, pools of sera were assessed for their ability to recognize the targeted protein at the surface of viable bacterial cells using flow cytometry (FACS analysis).

The individual sera raised to the constructs were first assayed for bactericidal activity against the homologous strain MC58. Results are expressed in terms of (i) GMTs (geometric mean titers), (ii) number of responders exhibiting a bactericidal titer superior or equal to 16 and, (iii) seroconversion compared to the negative control (fold-increase). Results are shown in Table 4 below. As used in the tables and Figures herein, the terms ‘seroconversion’, ‘seroconversion compared to the control’, ‘seroconversion compared to the corresponding buffer’ and ‘fold-increase’ are to be considered equivalent.

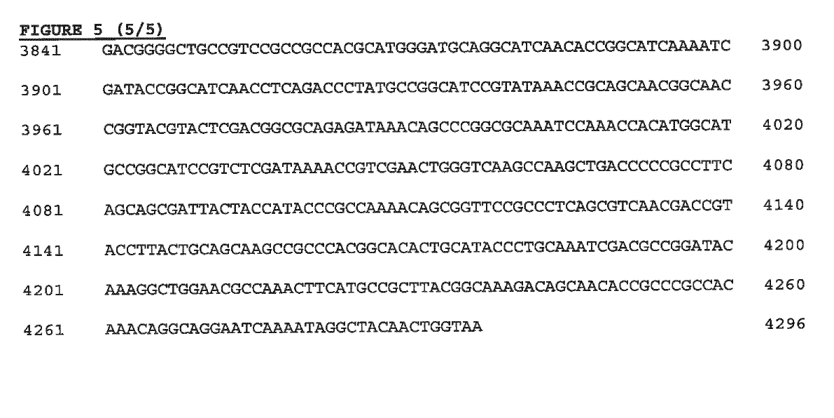

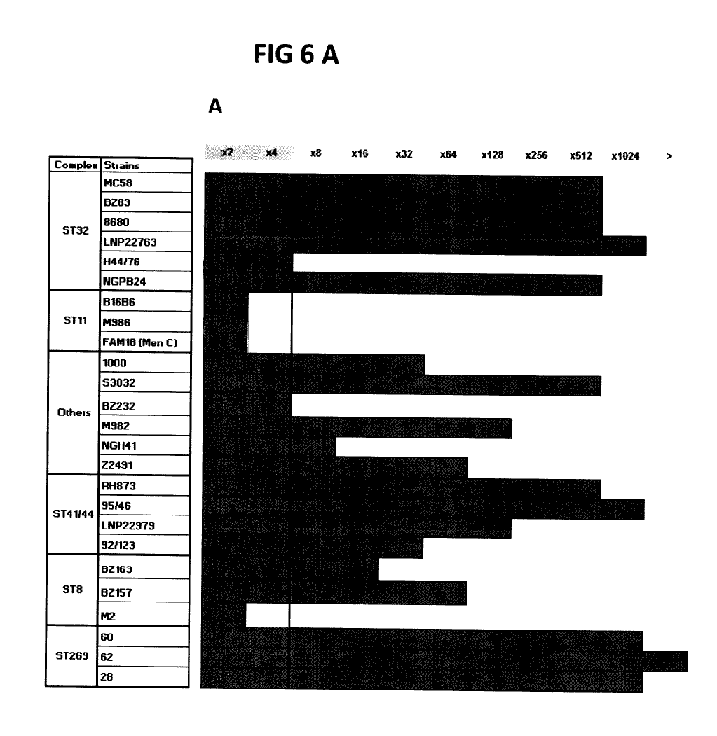

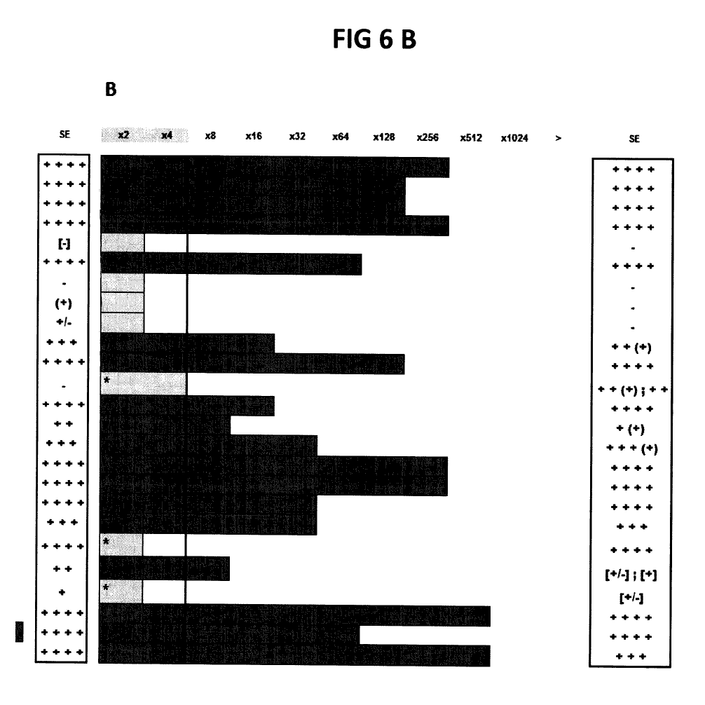

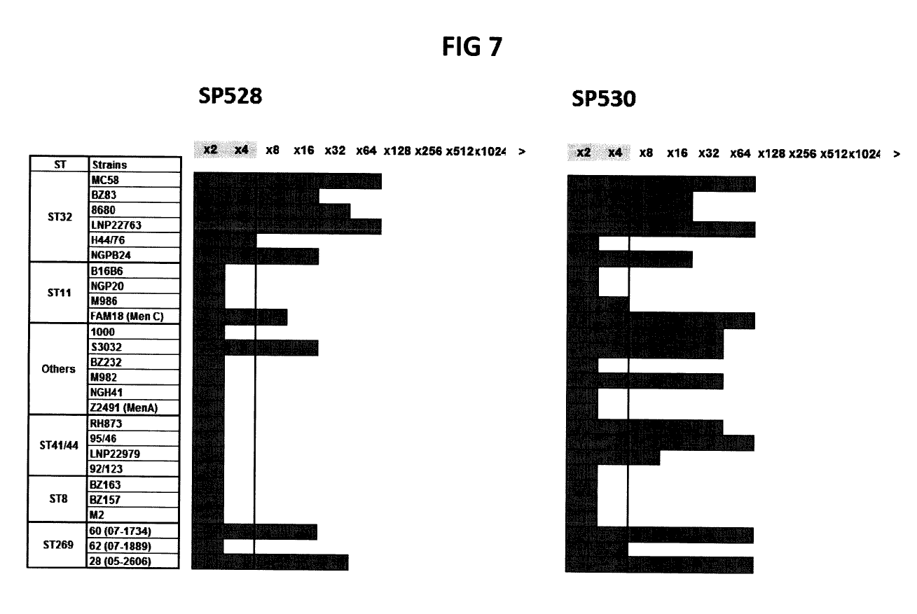

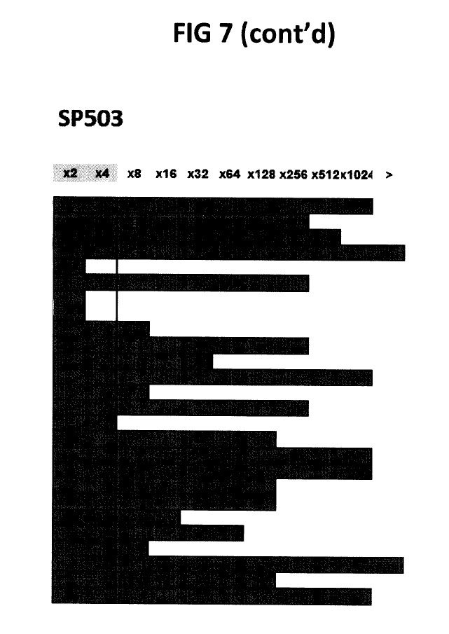

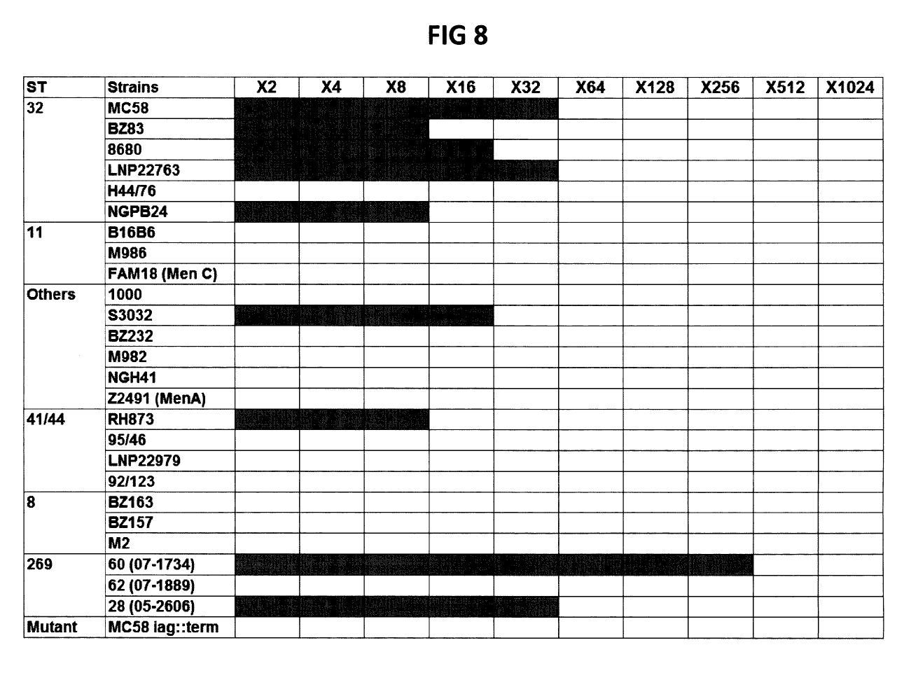

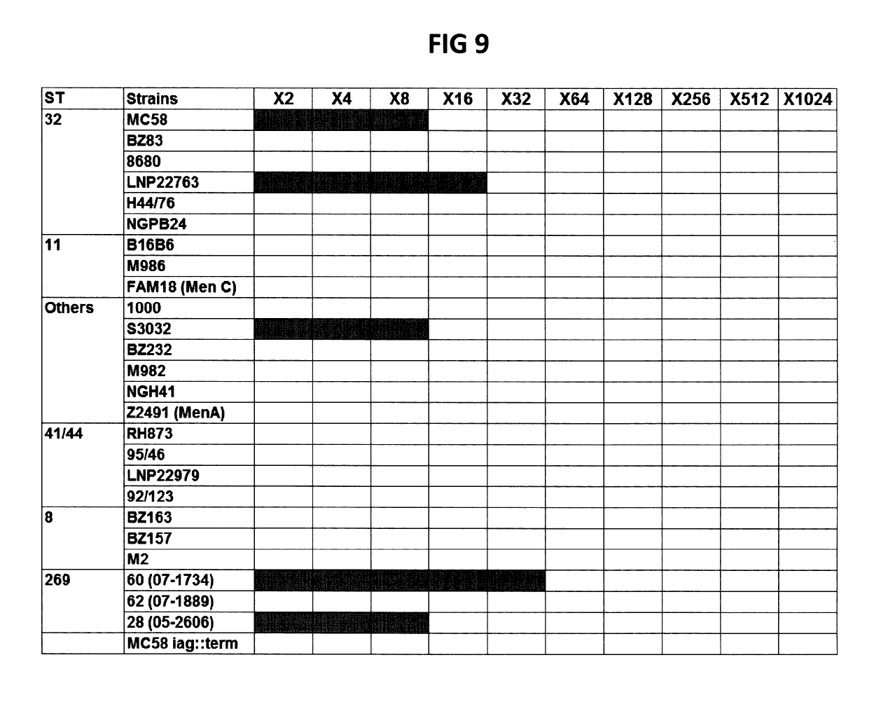

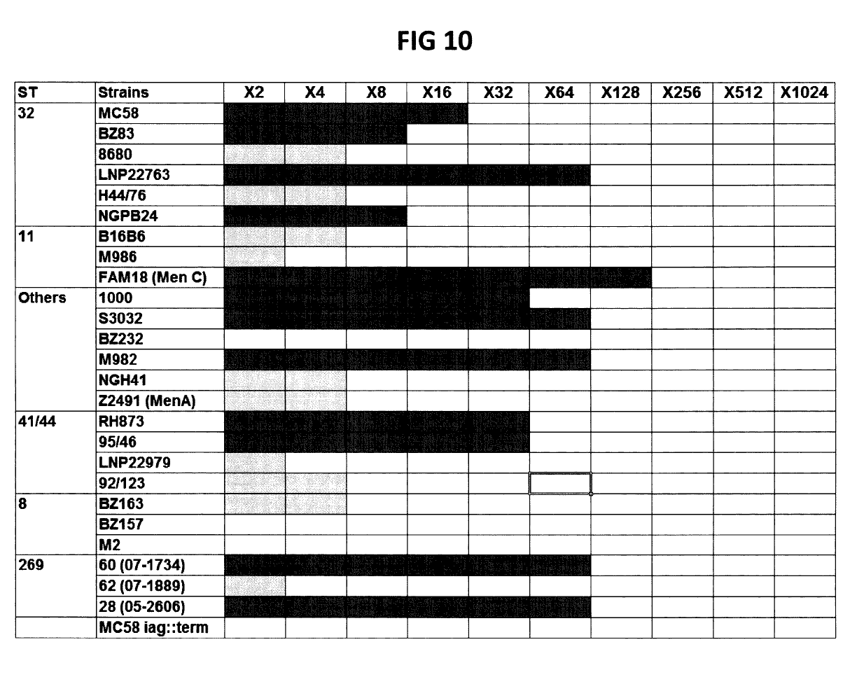

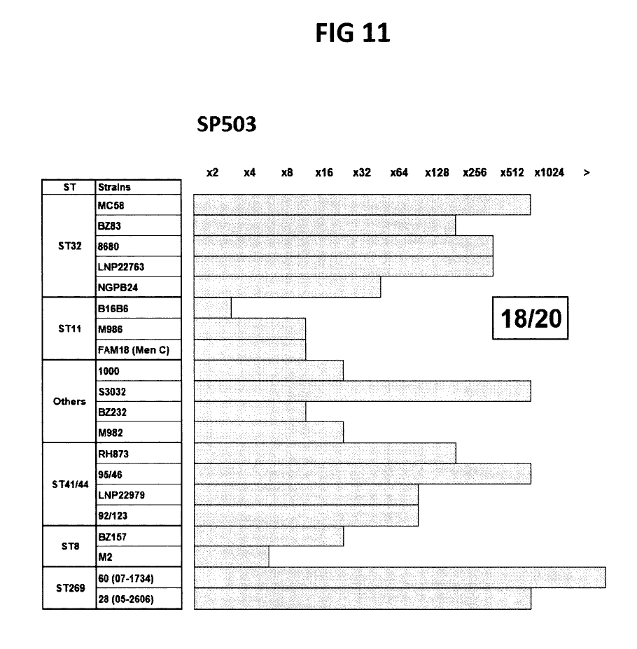

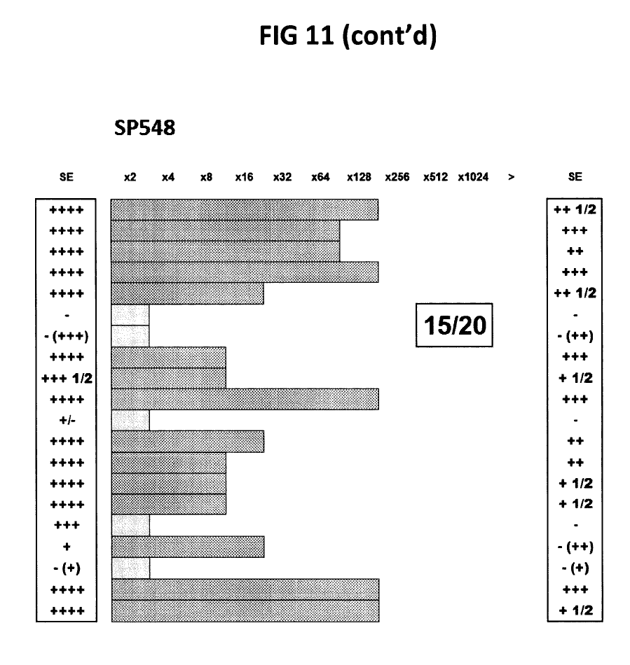

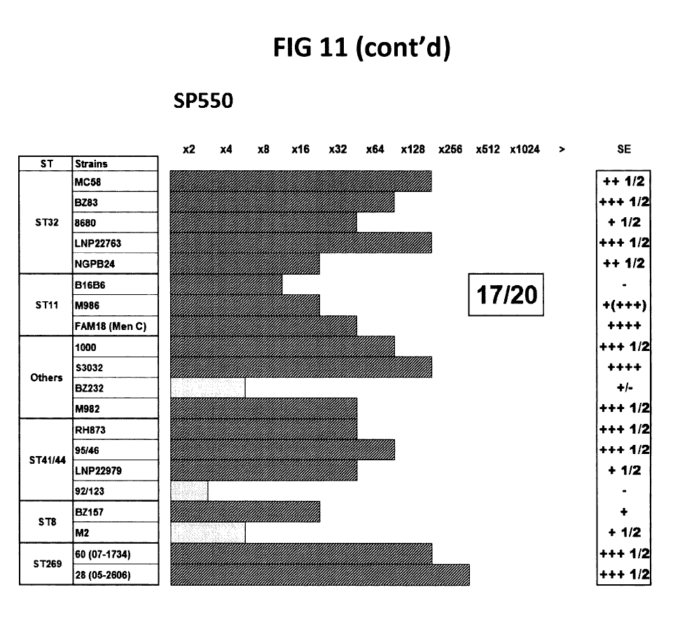

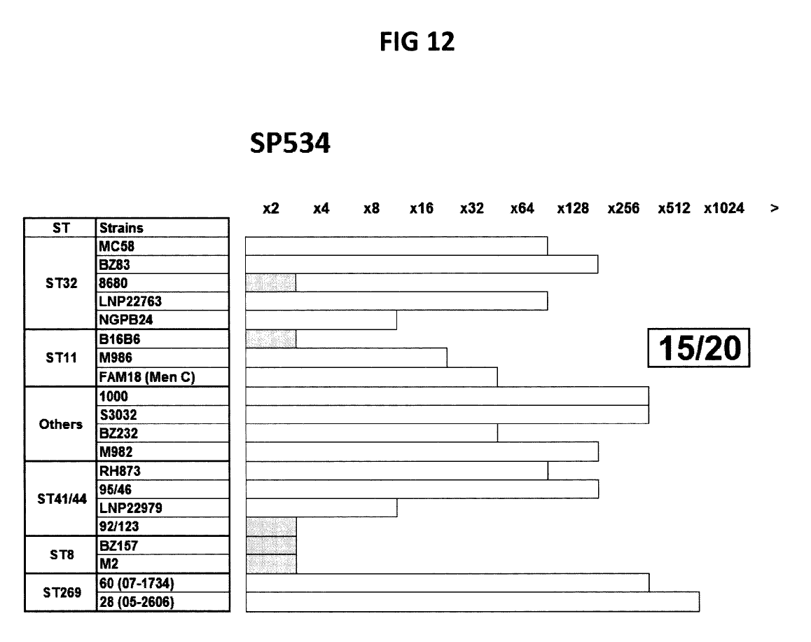

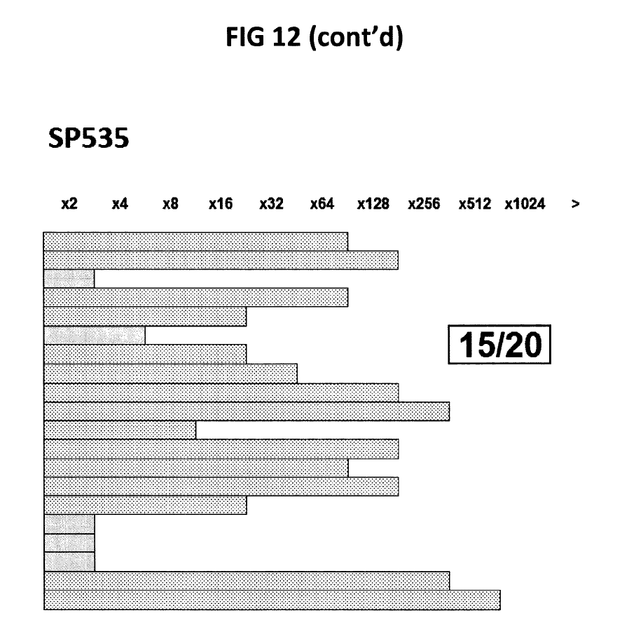

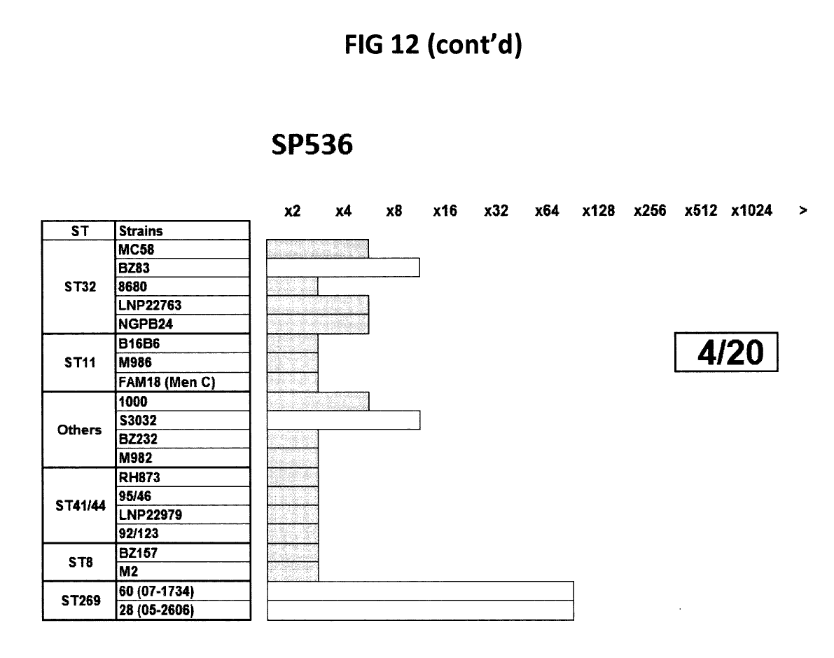

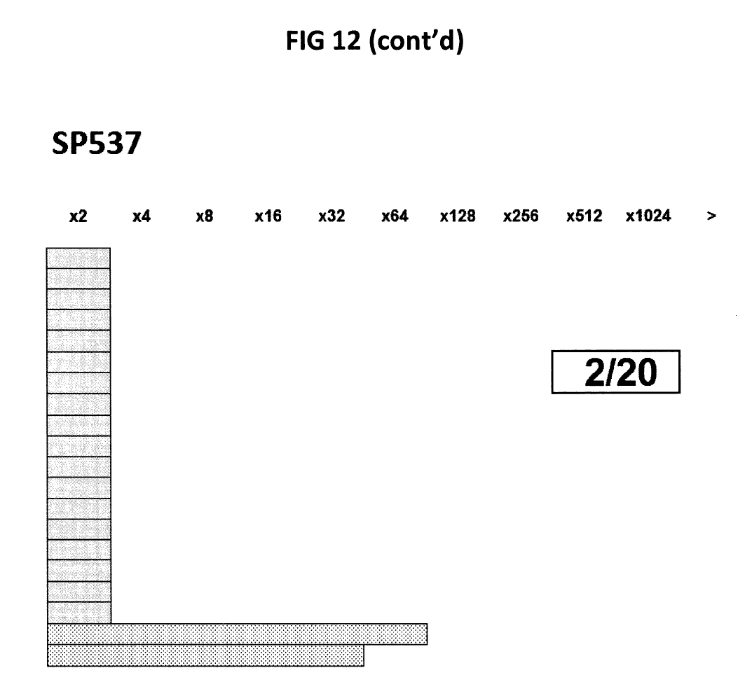

The constructs were also assayed for bactericidal activity against a panel of heterologous strains (Cross-bactericidal activity). Results are expressed in terms of seroconversion compared to a negative control (fold-increase). It is considered that cross-bactericidal activity is encountered when the fold-increase is superior or equal to 8. Results are summarized in Table 5 and detailed in FIG. 6 (Exp. A & B SP503), FIG. 7 (Exp C SP503-528-530); FIGS. 8, 9 and 10 (Exp C, respectively SP531, SP532 and SP533), FIG. 11 (Exp D SP503-548-550) and FIG. 12 (Exp E SP 534-535-536-537).

TABLE 4
SBA against the homologous strain MC58

| | | | | Homologous SBA data | | | | | | |
|---|---|---|---|---|---|---|---|---|---|---|
| | | | | | GMT (% of responders ≥ 16) | | | | | |
| | | | His- | Fold- | Construct | | Control | | Control | |
| Experiment | Constructs | | tag | increase | under test | | 1 | | 2 | |
| A | SP9 | IgA1P (28- | N-ter | X 1.2 | 2.8 | (0%) | 2.3 | (0%) | 2.6 | (0%) |
| | | 972) Ser 267 | | | | | | | | |
| | SP502 | IgA1P (28- | N-ter | X 180.8 | 415.9 | (100%) | | | | |
| | | 1584) Ser 267 | | | | | | | | |
| | SP503 | IgA1P (28- | N-ter | X 238.6 | 548.7 | (100%) | | | | |
| | | 1584) Val 267 | | | | | | | | |
| C | SP503 | IgA1P (28- | N-ter | X 79.0 | 955.4 | (100%) | 12.1 | (30%) | 4.9 | (10%) |
| | | 1584) Val 267 | | | | | | | | |
| | SP528 | IgA1P (28- | N-ter | X 9.1 | 109.7 | (100%) | | | | |
| | | 1005) Val 267 | | | | | | | | |
| | SP530 | IgA1P (28- | N-ter | X 4 | 48.5 | (100%) | | | | |
| | | 966) - App | | | | | | | | |
| | | (1061-1187) | | | | | | | | |
| | | Val 267 | | | | | | | | |
| | SP531 | IgA1P (28- | N-ter | X 4.9 | 59.7 | (90%) | | | | |
| | | 966) - Ausl | | | | | | | | |
| | | (974-1161) | | | | | | | | |
| | | Val 267 | | | | | | | | |
| | SP532 | IgA1P (28- | N-ter | X 1.72 | 21.1 | (60%) | | | | |
| | | 966) - App | | | | | | | | |
| | | (1061-1224) | | | | | | | | |
| | | Val 267 | | | | | | | | |
| | SP533 | IgA1P (28- | N-ter | X 3.5 | 42.2 | (80%) | | | | |
| | | 966) - Ausl | | | | | | | | |
| | | (974-1198) | | | | | | | | |
| | | Val 267 | | | | | | | | |
| D | SP503 | IgA1P (28- | N-ter | X 157 | 362 | (100%) | 2.3 | (0%) | 2.5 | (0%) |
| | | 1584) Val 267 | | | | | | | | |
| | SP548 | IgA1P (28- | C-ter | X 111 | 256 | (100%) | | | | |
| | | 1005) Val 267 | | | | | | | | |
| | SP550 | IgA1P (28- | C-ter | X 111 | 256 | (100%) | | | | |
| | | 966) - App | | | | | | | | |
| | | (1061-1187) | | | | | | | | |
| | | Val 267 | | | | | | | | |
| E | SP534 | App (43-1224) | N-ter | X 16.2 | 42.2 | (80%) | 2.6 | | 3.0 | |
| | | Ser 267 | | | | | | | | |
| | SP535 | App (43-1224) | N-ter | X 32.5 | 84.4 | (90%) | | | | |
| | | Val 267 | | | | | | | | |
| | SP536 | Ausl (26- | N-ter | X 3 | 8.0 | (30%) | | | | |
| | | 1198) Ser 241 | | | | | | | | |
| | SP537 | Ausl (26- | N-ter | X 1.6 | 4.3 | (20%) | | | | |
| | | 1198) Val241 | | | | | | | | |

Results are discussed in more details below:

Mutated, Truncated and/or Hybrid IgA1P

### SBA Against the Homologous Strain

A first set of truncated IgA1Ps, less hydrophobic than natural complete IgA1P, were produced and tested for SBA with the aim of determining the extent of truncation that would lead to positive SBA. Therefore, a short IgA1P was made the amino acid sequence of which corresponds to the protease domain with a deletion of about 30 amino acids at its C-terminal extremity (construct SP9). A much longer IgA1P was also made the amino acid sequence of which corresponds to the full-length IgA1P sequence deleted of all beta sheets except the 2 first ones (construct SP502). In addition to this, the catalytic site was inactivated by replacing Ser 267 with Alanine in SP502, leading to SP503.

Results are shown in Section A of Table 4. They showed that (i) the removal of all but the 2 first beta sheets was not detrimental to SBA and (ii) a truncated IgA1P should at least contain the entire protease domain or, if not, contain additional sequence corresponding to the alpha-peptide e.g., the alpha-peptide of another serine-protease.

These findings and hypotheses were further tested in second and third experiments for which additional truncated IgA1Ps were made, all of which bearing the mutation Ser 267 Val:

(i) Truncated IgA1P, the amino acid sequence of which corresponds exclusively to the entire protease domain (SP528, SP548).

(ii) Truncated IgA1P, the amino acid sequence of which corresponds to the protease domain with a deletion of about 35 amino acids at its C-terminal extremity, further fused to the MC58 App 1061-1187 sequence (SP530, SP550) or the MC58 AusI 974-1161 sequence (SP531) which both corresponds to the alpha-peptide domain.

(iii) Truncated IgA1P, the amino acid sequence of which corresponds to the protease domain with a deletion of about 35 amino acids at its C-terminal extremity, further fused to the MC58 App 1061-1224 sequence (SP532) or the MC58 AusI 974-1198 sequence (SP533) which both corresponds to the alpha-peptide domain followed by the first two beta-strands.

These constructs were assayed for SBA together with SP503 for which the best results were to be seen in the first experiment. Results are to be seen in Sections C and D of Table 4. They reveal that a truncated IgA1P corresponding to the entire protease domain is effective in raising SBA. In addition to this, the fusion of a partial IgA1P protease domain to an App or AusI structure equivalent to the IgA1P alpha-peptide domain results in hybrids exhibiting positive SBA. Interestingly, SP528 and SP530 exhibit a modest fold-increase although they induce a bactericidal titer >16 in 100% of mice. Moving the His-tag to the C-ter (SP548 & SP550 respectively) allows significant improvement.

Mutated, Truncated and/or Chimeric IgA1P

### SBA Against Heterologous Strains

In a first set of two independent experiments A & B, the cross-bactericidal activity of mouse pooled sera raised against SP503 was assayed against a panel of 25 strains including the homologous strain MC58, most of them being spread over 5 major epidemiological clusters (ST32, ST11, ST41/44, ST8, ST269). Strains were cultured in BHI+desferal, 2 hr 30 except strain NGH41, cultured for 4 hrs in BHI agar. Results are shown in FIG. 6 and reveal high cross-SBA coverage. The expression of the IgA1P antigen at the bacterial surface as quantified by FACS tends to correlate with the fold-increase factor measured in SBA.

In a second and similar experiment C, SP503 was assayed together with SP528 and SP530 for cross-SBA against a panel of 26 strains including the homologous strain MC58. Results are shown in FIG. 7 and Table 5 above. Percentages of strains killed over the 26 strains tested are 85, 35 and 54% respectively. SP531, SP532 and SP533 were also tested in the same experiment. Results are shown in FIGS. 8, 9 and 10 respectively, and in Table 5 above. While antisera raised against SP531, SP532 and SP533 only show modest bactericidal activity against the homologous strain, they were able to cross-react with strains of the ST269 complex and therefore are of potential interest.

In a third and similar experiment D, SP503 was assayed together with SP548 and SP550 for cross-SBA against a panel of 20 strains including the homologous strain MC58. Results are shown in Table 5 above and in FIG. 11. Percentages of strains killed over the 20 strains tested are 90, 75 and 85% respectively.

Truncated and/or Mutated App & AusI

### Homologous & Heterologous SBA

In Experiment E, truncated App constructs SP534 and SP535 as well as truncated AusI constructs SP536 and SP537 were tested for SBA against a panel of 20 strains. Results are shown in FIG. 12 and Table 5 above.

Antisera raised against truncated App either mutated or not (SP534 & SP535) exhibit significant bactericidal activity against the homologous strain and high cross-SBA coverage. While antisera raised against truncated AusI, mutated or not (SP536 & SP537), do not show any bactericidal activity against the homologous strain, they are able to cross-react with strains of the ST269 complex and therefore are of potential interest.

### REFERENCES

Lomholt et al., Mol. Microb. (1995) 15 (3): 495
Vitovski & Sayers, Infect. Immun. (2007) 75 (6): 2875
Ulsen & Tommassen, FEMS Microbiol. Rev. (2006) 30 (2): 292
WO 90/11367
Tettelin et al., Science (March 2000) 287: 1809
Gripstra et al., Res. in Microbiol. (2013) 164: 562
Pohlner et al., Nature (1987) 325: 458
van Ulsen et al., FEMS Immunol Med. Microb. (2001) 32: 53
Serruto et al., Mol. Microb. (2003) 48 (2): 323
van Ulsen et al., Microbes & Infection (2006) 8: 2088
Turner et al., Infect. Immun. (2006) 74 (5): 2957
Henderson et al., Microbiol. Mol. Biol. Rev. (2004) 68 (4): 692
Smith et al., J. Mol. Biol. (1981) 147: 195
Altschul et al., (1990) J. Mol. Biol., 215: 403
Roussel-Jazédé et al., Infect Immun. (2010) 78 (7): 3083
van Ulsen P. et al., Mol Microbiol. (November 2003) (3): 1017
Serruto et al., PNAS February 2010 107 (8): 3770
Vitovski et al., (1999) FASEB J. 13: 331
Sambrook et al., Molecular Cloning: A Laboratory Manual, New York: Cold Spring Harbor Press, 1989
WO 94/00153
U.S. Pat. No. 6,113,918
U.S. Pat. No. 6,303,347
WO 98/50399
US 2003/0153532
WO2007/005583
WO 07/006939
U.S. Pat. No. 7,683,200
WO 07/080308
Rokbi et al., Clin. Diagnostic Lab. (1997) 4 (5): 522

1. An isolated polypeptide comprising or consisting of:
(I):
(A) a fragment of a full-length mature trypsin-like serine protease auto-transporter of N. meningitidis, said fragment consisting of:
(i) a protease domain of a trypsin-like serine protease auto-transporter of N. meningitidis; or
(ii) a protease domain and all or part of an α-peptide domain of a trypsin-like serine protease auto-transporter of N. meningitidis; or
(iii) a protease domain, an α-peptide domain and a part of a β-domain of a trypsin-like serine protease auto-transporter of N. meningitidis;
or
(B) a mutant of said fragment (A) which lacks or has reduced trypsin-like serine protease activity and/or does not contain any cleavage site able/susceptible to be cleaved by a trypsin-like serine protease;
wherein said polypeptide under (A) or (B) does not comprise the said full-length mature trypsin-like serine protease auto-transporter of N. meningitidis;
or
(II)
a first fragment fused to a second fragment:
(1) said first fragment consisting of:
a protease domain or a protease sub-domain of a first trypsin-like serine protease auto-transporter of N. meningitidis, or
a mutant of a protease domain or a protease sub-domain of a first trypsin-like serine protease auto-transporter of N. meningitidis which lacks or has reduced trypsin-like serine protease activity and/or does not contain any cleavage site able/susceptible to be cleaved by a trypsin-like serine protease,
(2) said second fragment consisting of:
an α-peptide domain of a second trypsin-like serine protease auto-transporter of N. meningitidis;
wherein the first and second trypsin-like serine protease auto-transporters are different; and
wherein the C-terminus of the first fragment is fused to the N-terminus of the second fragment,
wherein said polypeptide does not comprise the said full-length mature trypsin-like serine protease auto-transporter of N. meningitidis.

2. An isolated polypeptide according to claim 1, wherein said fragment consists of the protease domain of the trypsin-like serine protease auto-transporter of N. meningitidis which is IgA1P, App or AusI.

3. An isolated polypeptide according to claim 1, wherein said fragment consists of a protease domain and all or part of an α-peptide domain of the trypsin-like serine protease auto-transporter of N. meningitidis which is IgA1P, App or AusI.

4. An isolated polypeptide according to claim 1, wherein said fragment consists of the protease domain, the α-peptide domain and a part of a β-domain, of the trypsin-like serine protease auto-transporter of N. meningitidis which is IgA1P, App or AusI.

5. An isolated polypeptide according to claim 1, comprising or consisting of a first fragment fused to a second fragment wherein said first fragment consists of a protease sub-domain of said first trypsin-like serine protease auto-transporter and said second fragment consist of an α-peptide domain of said second trypsin-like serine protease auto-transporter.

6. An isolated polypeptide according to claim 5, wherein said first trypsin-like serine protease auto-transporter is IgA1P and said second trypsin-like serine protease auto-transporter is App or AusI.

7. An isolated polypeptide according to claim 2, which has an amino acid sequence having at least 90% identity with the amino acid sequence of the IgA1P of N. meningitidis MC58 shown in SEQ ID NO: 1 starting from position 27, 28, 29, 30, 31 or 32 and ending at position 1002, 1003, 1004, 1005, 1006, 1007 or 1008.

8. An isolated polypeptide according to claim 4, which has an amino acid sequence having at least 90% identity with the amino acid sequence of the IgA1P of N. meningitidis MC58 shown in SEQ ID NO: 1 starting from position 27, 28, 29, 30, 31 or 32 and ending at position 1580, 1581, 1582, 1583, 1584, 1585, 1586, 1587 or 1588.

9. An isolated polypeptide according to claim 4, which has an amino acid sequence having at least 90% identity with the amino acid sequence of the App of N. meningitidis MC58 shown in SEQ ID NO: 3 starting from position 40, 41, 42, 43, 44, 45 or 46 and ending at position 1220, 1220, 1221, 1223, 1224, 1225, 1226 or 1227.

10. An isolated polypeptide according to claim 4, which has an amino acid sequence having at least 90% identity with the amino acid sequence of the AusI of N. meningitidis MC58 shown in SEQ ID NO: 3 starting from position 26, 27, 28, 29, 30 or 31 and ending at position 1194, 1195, 1196, 1197, 1198, 1199, 1200, 1201 or 1202.

11. An isolated polypeptide according to claim 1, comprising or consisting of a first fragment fused to a second fragment wherein said first fragment has at least 90% identity with an amino acid sequence of the IgA1P of N. meningitidis MC58 shown in SEQ ID NO: 1 starting from position 27, 28, 29, 30, 31 or 32 and ending at position 1002, 1003, 1004, 1005, 1006, 1007 or 1008;
and wherein said second fragment has at least 90% identity with an amino acid sequence of
(i) App of N. meningitidis MC58 shown in SEQ ID NO: 3 starting from position 1057, 1058, 1059, 1060, 1061 or 1062 and ending at a position between 1170 and 1204 inclusive; or
(ii) AusI of N. meningitidis MC58 shown in SEQ ID NO: 5 starting from position 969, 970, 971, 972, 973, 974, 975, 976, 977, 978, 979 or 980 and ending at a position between 1131-1177 inclusive.

12. An isolated polypeptide according to claim 7, wherein said first fragment has at least 90% identity with an amino acid sequence of the IgA1P of N. meningitidis MC58 shown in SEQ ID NO: 1 starting from position 27, 28, 29, 30, 31 or 32 and ending at position 960, 961, 962, 963, 964, 965, 966, 967, 968, 969, 9701, 971, 972, 973, 974, 975, 976, 977, 978, 979 or 980;
and wherein said second fragment has at least 90% identity with an amino acid sequence of
(i) App of N. meningitidis MC58 shown in SEQ ID NO 3: starting from position 1057, 1058, 1059, 1060, 1061 or 1062 and ending at a position between 1170 and 1204 inclusive; or
(ii) AusI of N. meningitidis MC58 shown in SEQ ID NO 5: starting from position 969, 970, 971, 972, 973, 974, 975, 976, 977, 978, 979 or 980 and ending at a position between 1131 and 1177 inclusive.

13. An isolated polypeptide according to claim 8, wherein said first fragment has at least 90% identity with an amino acid sequence of the IgA1P of N. meningitidis MC58 shown in SEQ ID NO: 1 starting from position 27, 28, 29, 30, 31 or 32 and ending at position 960, 961, 962, 963, 964, 965, 966, 967, 968, 969, 9701, 971, 972, 973, 974, 975, 976, 977, 978, 979 or 980;
and wherein said second fragment has at least 90% identity with an amino acid sequence of
(i) App of N. meningitidis MC58 shown in SEQ ID NO 3: starting from position 1057, 1058, 1059, 1060, 1061 or 1062 and ending at a position between 1170 and 1204 inclusive, and preferably at a position 1183, 1184, 1185, 1186, 1187, 1188, 1189, 1190 or 1191; or
(ii) App of N. meningitidis MC58 shown in SEQ ID NO 3: starting from position 1057, 1058, 1059, 1060, 1061 or 1062 and ending at a 1220, 1221, 1222, 1223, 1224, 1125, 1226, 1227, or 1228; or
(iii) AusI of N. meningitidis MC58 shown in SEQ ID NO 5: starting from position 969, 970, 971, 972, 973, 974, 975, 976, 977, 978, 979 or 980 and ending at a position between 1131 and 1177 inclusive, and preferably at a position 1157, 1158, 1159, 1160, 1161, 1162, 1163, 1164, or 1165; or
(iv) AusI of N. meningitidis MC58 shown in SEQ ID NO 5: starting from position 969, 970, 971, 972, 973, 974, 975, 976, 977, 978, 979 or 980 and ending at a position between 1194, 1195, 1196, 1197, 1198, 1199, 1200, 1201 or 1202.

14. An isolated polypeptide according to claim 1, wherein the serine protease activity is inactivated by amino acid substitution in the catalytic triad or in the serine protease motif.

15. An isolated polypeptide according to claim 14, wherein the amino acid substitution occurs at the serine residue of the catalytic triad.

16. A nucleic acid encoding a polypeptide according to claim 1.

17. A vector comprising a nucleic acid according to claim 16.

18. A host cell comprising a nucleic acid according to claim 16.

19. (canceled)

20. (canceled)

21. A vaccine composition comprising a polypeptide according to claim 1.

22. A method of production of a polypeptide according to claim 1, the method comprising expression of said polypeptide from a vector comprising a nucleic acid encoding the polypeptide of claim 1.

23. An isolated polypeptide according to claim 4, wherein said fragment consists of the protease domain, the α-peptide domain and the two first β-sheets of the β-domain, of the trypsin-like serine protease auto-transporter of N. meningitidis which is IgA1P, App or AusI.

24. A host cell comprising a vector according to claim 17.

25. A method of treating N. meningitidis B infection in a subject, the method comprising administering to the subject an effective amount of a polypeptide according to claim 19.


SEQUENCE LISTING (6 sequences)

SEQ ID NO: 1 - protein, 1815 aa, Neisseria meningitidis
MKTKRFKINAISLSIFLAYALTPYSEAALVRDDVDYQIFRDFAENKGKFFVGATDLSVKNKQGQNIGNALSNVPMIDFSVADVNRRTLTVIDPQYAVSVKHVKGDEISYYGHHNGHLDVSNDENEYRSVAQNDYEPNKNWHHGNQGRLEDYNMARLNKFVTEVAPIAPTSAGGGVETYKDKNRFSEFVRVGAGTQFEYNSRYNMTELSRAYRYAIAGTPYQDVNVTSNLNQEGLIGFGDNSKHHSPEKLKEVLSQNALTNYAVLGDSGSPLFAYDKQEKRWVFLGAYDYWAGYQKNSWQEWNIYKKEFADEIKQRDNAGTIKGNGEHHWKTTGTNSHIGSTAVRLANNERDANNGQNVTFENNGTLVLDQNINQGAGGLFFKGDYTVKGINNDITWLGAGIDVADGKKVVWQVKNPNGDRLAKIGKGTLEINGTGVNQGQLKVGDGTVILNQQADADKKVQAFSQVGIVSGRGTLVLNSSNQINPDNLYFGFRGGRLDANGNDLTFEHIRNVDEGARIVNHNTGHASTITLTGKSLITDPKTISIHYIQNNDDDDAGYYYYRPRKPIPQGKDLYFKNYRYYALKSGGSVNAPMPENGQTENNDWILMGSTQEEAKKNAMNHKNNQRISGFSGFFGEENGKGHNGALNLNFNGKSAQNRFLLTGGTNLNGKISVTQGNVLLSGRPTPHARDFVNKSSARKDAHFSKNNEVVFEDDWINRTFKATEIAVNQSASFSSGRNVSDITANITATDNAKVNLGYKNGDEVCVRSDYTGYVTCNTGNLSDKALNSFGATQINGNVNLNQNAALVLGKAALWGQIQGQGNSRVSLNQHSKWHLTGDSQVHNLSLADSHIHLNNASDAQSANKYHTLKINHLSGNGHFHYLTHLAKNLGDKVLVKESASGHYQLHVQDKTGEPNQEGLNLFDASSVQDRSRLSVSLANNHVDLGALRYTIKTENGITRLYNPYAENRRRVKPAPSPATNTASQAQKATQTDGAQIAKPQNIVVAPPSPQANQAEEAKRQQAKAEQVKRQQAEAERKSAELAKQKAEAEREARELATRQKAEQERSSAELARRHEKEREAAELSAKQKVEAEREAQALAVRRKAEAEEAKRQAAELARRHEKEREAAELSAKQRVGEEERRQTAQSQPQRRKRRAAPQDYMAASQDRPKRRGHRSVQQNNVEIAQAQAELARRQQEERKAAELLAKQRAEAEREAQALAARRKAEAEEAKRQAAELAHRQEAERKAAELSANQKAAAEAQALAARQQKALARQQEEARKAAELAVKQKAETERKTAELAKQRAAAEAAKRQQEARQTAELARRQEAERQAAELSAKQKAETDREAAESAKRKAEEEEHRQAAQSQPQRRKRRAAPQDYMAASQNRPKRRGRRSTLPAPPSPSFDSSAYAAPRALHNPDWYENDYEEIPLDALEDENVSESVDTSDKQPQDNTELHEKYENDYEEIPLDALEDEDVSESVDTSDKQPQDNTELHEKVETVSLQPRAAQPRAQAATQLQAQAAAQADAVSTNTNSALSDAMASTQSILLDTGASLTRHIAQKSRADAEKNSVWMSNTGYGRDYASAQYRRFSSKRTQTQIGIDRSLSENMQIGGVLTYSDSQHTFDQASGKNTFVQANLYGKYYLNDAWYVAGDIGAGSLRSRLQTQQKANFNRASIQTGLTLGNTLKINQFEIVPSAGIRYSRLSSADYKLGNDSVKVSSMSVKTLTAGLDFAYRFKVGNLTVKPLLSAAYFANYGKGGVNVGGNSFVYKADNQQQYSAGAALLYRNVTLNVNGSITKGKQLEKQKSGQIKIQIRF

SEQ ID NO: 2 - DNA, 5448 bp, Neisseria meningitidis
  source 1..5448: /mol_type=“unassigned DNA” /organism=“Neisseria meningitidis”
atgaaaaccaaacgttttaaaattaacgccatatccttatccatctttcttgcctatgcccttacgccatactcagaagcggcattggtcagagacgatgtcgattatcaaatattccgtgactttgcagaaaacaaaggcaaattttttgtcggcgcaaccgatttatcagtgaaaaacaaacaaggtcaaaacatcggcaacgcattatccaacgtaccgatgattgattttagcgttgcagatgtcaacagacgtacattaaccgtcatcgatccccagtatgccgtcagcgtcaaacatgtaaaaggagacgaaatctcttattacgggcatcacaatgggcacttagatgttagcaatgatgaaaacgaatatcgttcagttgcacaaaatgactacgaaccaaataaaaattggcatcacggtaatcaaggtcgtcttgaagactacaacatggcacgccttaataaattcgtaacagaagtcgcacctattgcaccaaccagtgcaggcggcggcgttgaaacctataaagataaaaaccgtttttctgagtttgtgcgagttggagcaggtacgcaatttgaatataacagccgctacaatatgacagagctttcacgagcttatcgttatgccattgcaggtacgccttatcaagacgtgaatgtcacatcgaatctaaatcaagaaggcttgattggcttcggtgataattcaaaacatcattccccagaaaaactcaaagaagttttatctcaaaacgcattgactaactatgctgtgttaggcgatagcggttcaccattatttgcttatgataaacaagaaaaacgctgggtctttttaggtgcttacgattattgggcaggctatcaaaaaaactcttggcaagaatggaatatctataaaaaagaatttgcagatgaaatcaaacaacgcgataacgccggcaccatcaaaggtaatggagaacatcattggaaaaccacgggcacaaacagccatatcggttcgacagcggtaaggcttgccaacaatgaaagggatgcaaacaacggacagaatgttacctttgaaaacaacggcactttggtattggatcaaaacatcaaccaaggcgcgggcggtctgtttttcaaaggcgattacacagtcaaaggtataaataatgacatcacttggctaggtgcggggattgatgttgccgacggcaaaaaagtcgtttggcaagtcaaaaatccgaatggcgacagattggcaaaaatcggcaaaggcacattagaaataaacggcacaggcgttaaccaagggcaattaaaagtcggcgacggtacggttattctgaatcaacaagccgatgccgacaaaaaagtccaggctttctcccaagtcggcattgtcagcggacgcggtacattggtattaaatagttcaaatcagattaatcccgataacctatatttcggtttccgtggcggtcgtttggatgccaatggcaatgacttgacttttgaacacatccgcaacgtggatgaaggcgcgcgcattgtcaaccacaacacaggccacgcctccacaatcacgctaacgggtaaatctttgattaccgatcccaaaaccatctctattcattatattcaaaataatgatgatgacgatgctggttattactattaccgccctaggaaaccgattccacaaggcaaagatctttatttcaaaaactaccgttattacgccctaaaatccggcggcagcgtgaacgcaccgatgcccgaaaacggacaaacggaaaataacgactggattttaatgggcagcacacaagaagaagccaagaaaaacgcgatgaaccacaaaaacaatcagcgtattagcggttttagcggtttctttggcgaagaaaacggaaaaggacataacggtgcattaaaccttaatttcaacggcaaaagcgcgcaaaaccgtttcttgttaacaggcggcactaatttaaacggaaaaataagcgtaactcaaggcaatgtcttgttatcaggtcgtccaacaccacacgcaagagattttgtgaacaaatcttcagcccgaaaagacgcacatttctccaaaaacaatgaagtcgtatttgaagacgactggataaaccgcacattcaaagccacagaaattgcggttaatcaatccgcatcattctcttccggcagaaatgtatccgacatcaccgccaacatcaccgcaacagacaacgccaaagtaaatttgggttacaaaaacggcgatgaggtttgcgtgcgctcggactataccggttacgttacctgcaacacaggcaacttatccgataaggctttaaacagctttggagcgacacagattaacggcaatgtgaatctgaaccagaatgcggcattggtcttgggcaaggctgcattatgggggcaaattcaagggcaaggaaacagccgtgtcagcctaaaccaacatagcaaatggcatttgaccggcgacagccaagtacacaatctgtcattggcggatagccatattcatttgaacaatgcctccgatgcgcaaagtgcaaataaataccacacgctcaaaatcaatcatttatccggtaacgggcattttcattatctgacgcacctggcgaaaaatcttggggataaagtgcttgtgaaggaatccgcatccggtcattatcagctccatgttcaagataaaacaggcgaacccaatcaggaagggctgaatctctttgatgcatcatccgtacaagaccgctcccgcctttctgtttccttggcgaataaccatgtagatttaggggcattgcgttatacaatcaaaacagaaaacggtattacccgattgtacaatccttatgccgagaaccgccgccgagtcaaaccggccccgtctcctgccacaaacacggcttctcaagcacaaaaggcaacacaaacggacggtgcacaaattgccaagcctcaaaatatcgtcgtcgcaccgcctagcccgcaggcaaatcaagccgaagaagcaaaacgccagcaagcaaaagcggagcaagtgaagcgtcagcaagcagaagcagagcgtaaatcggcagagttagcaaaacaaaaagcagaagccgaacgcgaagcaagagagctggcgacccgccaaaaagcagaacaagaacgcagcagcgccgaacttgcacgtcgacatgaaaaagaacgcgaagcggcagagttgtcggcaaaacaaaaagtcgaagccgaacgtgaggctcaagcattggcggtacgccgaaaagcagaagccgaagaggcaaaacgccaagctgcagagcttgcacgccggcatgaaaaagaacgcgaagcggcagagttgtcggcaaaacaaagggtcggggaagaggaacgccgtcaaacagcccaatcccagccgcaacgccgcaaacgccgtgccgcaccgcaggattatatggcagcttcccaagaccgtcctaaacgccgcggacaccgttcggtacagcaaaacaatgtggaaattgcccaagcccaagcagaacttgcgcgccgacagcaggaagaacgcaaagcggcagagttgttggcaaaacaaagggcagaagccgaacgtgaagctcaagcattggctgcgcgccgaaaagcagaagccgaagaggcaaaacgccaagctgccgaacttgcacatcgccaggaagcagaacgcaaagcggcagaattgtcggcaaaccaaaaagccgcagctgaagcccaagcattggcggcacgccaacaaaaagcccttgcgcgtcaacaggaagaagcacgaaaagctgcagagttagccgtcaaacaaaaagcggaaaccgaacgcaaaaccgccgaacttgccaagcaaagggcagcagccgaagcagcaaaacgccagcaagaagctcgccagaccgccgaacttgcacgtcgtcaagaagcggaacgccaagcagcagagttgtcggcaaaacaaaaagccgaaacagatcgagaagcggcagaatcggcgaaacgaaaagctgaggaagaagagcaccgtcaagcagcccaatcccagccgcaacgccgaaaacgccgtgccgcacctcaggattatatggcagcttcccaaaatcgtccgaaacgtcgcggacgcagatctactctgccggcaccgccctcgccatcatttgattcgtcagcttacgcagcacccagggccttgcataatccggactggtatgaaaatgactatgaagaaatccccttggatgcgttggaagatgaaaatgtatctgaatcagttgacacatcagacaaacagcctcaagacaatacggaacttcatgaaaaatatgaaaatgattatgaagaaatccccttggatgcgttggaagatgaagatgtatccgaatcggttgacacatcagacaaacagcctcaagacaatacggaacttcatgaaaaagttgagacagtgagtttgcaaccaagagccgcgcagccgcgagcccaagccgccactcagctgcaagcccaagccgccgcgcaagccgatgcagtcagcaccaataccaactcggctttatctgacgcaatggcaagcacgcaatctatcttgttggatacaggtgcttcattaacacggcacattgcacaaaaatcacgcgctgatgccgaaaaaaacagtgtttggatgtcgaacaccggttatggtcgtgattatgcttccgcacaatatcgccggtttagttcgaaacgcacgcaaacacaaatcggcattgaccgcagcttgtccgaaaatatgcagataggcggcgtattgacttactctgacagtcagcatacttttgatcaggcgagcggcaaaaatacttttgtgcaagccaacctttatggtaagtattatttaaatgatgcttggtatgtggccggcgatattggtgcaggcagcttgagaagccggttacaaacgcagcaaaaagcaaactttaaccgagcaagcatccaaaccggccttactttgggcaatacgctgaaaatcaatcaattcgagattgtccccagtgcggggatccgttacagccgcctgtcatctgcagattacaagttgggtaacgacagtgttaaagtaagttctatgtcagtgaaaacactaacggccggactggattttgcttatcggtttaaagtcggcaaccttaccgtaaaacccttgttatctgcagcttactttgccaattatggcaaaggcggcgtgaatgtgggcggtaattccttcgtctataaagcagataatcaacagcaatattcagcaggtgccgcgttactgtaccgtaatgttacattaaacgtaaatggcagtattacaaaaggaaaacaattggaaaaacaaaaatccggacaaattaaaatacagattcgtttctaa

SEQ ID NO: 3 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 4 - DNA, 4374 bp, Neisseria meningitidis
  source 1..4374: /mol_type=“unassigned DNA” /organism=“Neisseria meningitidis”
atgaaaacaaccgacaaacggacaaccgaaacacaccgcaaagccccgaaaaccggccgcatccgcttctcgcctgcttacttagccatatgcctgtcgttcggcattcttccccaagcctgggcgggacacacttatttcggcatcaactaccaatactatcgcgactttgccgaaaataaaggcaagtttgcagtcggggcgaaagatattgaggtttacaacaaaaaaggggagttggtcggcaaatcaatgacaaaagccccgatgattgatttttctgtggtgtcgcgtaacggcgtggcggcattggtgggcgatcaatatattgtgagcgtggcacataacggcggctataacaacgttgattttggtgcggaaggaagaaatcccgatcaacatcgttttacttataaaattgtgaaacggaataattataaagcagggactaaaggccatccttatggcggcgattatcatatgccgcgtttgcataaatttgtcacagatgcagaacctgttgaaatgaccagttatatggatgggcggaaatatatcgatcaaaataattaccctgaccgtgttcgtattggggcaggcaggcaatattggcgatctgatgaagatgagcccaataaccgcgaaagttcatatcatattgcaagtgcgtattcttggctcgttggtggcaatacctttgcacaaaatggatcaggtggtggcacagtcaacttaggtagtgaaaaaattaaacatagcccatatggttttttaccaacaggaggctcatttggcgacagtggctcaccaatgtttatctatgatgcccaaaagcaaaagtggttaattaatggggtattgcaaacgggcaacccctatataggaaaaagcaatggcttccagctggttcgtaaagattggttctatgatgaaatctttgctggagatacccattcagtattctacgaaccacgtcaaaatgggaaatactcttttaacgacgataataatggcacaggaaaaatcaatgccaaacatgaacacaattctctgcctaatagattaaaaacacgaaccgttcaattgtttaatgtttctttatccgagacagcaagagaacctgtttatcatgctgcaggtggtgtcaacagttatcgacccagactgaataatggagaaaatatttcctttattgacgaaggaaaaggcgaattgatacttaccagcaacatcaatcaaggtgctggaggattatatttccaaggagattttacggtctcgcctgaaaataacgaaacttggcaaggcgcgggcgttcatatcagtgaagacagtaccgttacttggaaagtaaacggcgtggcaaacgaccgcctgtccaaaatcggcaaaggcacgctgcacgttcaagccaaaggggaaaaccaaggctcgatcagcgtgggcgacggtacagtcattttggatcagcaggcagacgataaaggcaaaaaacaagcctttagtgaaatcggcttggtcagcggcaggggtacggtgcaactgaatgccgataatcagttcaaccccgacaaactctatttcggctttcgcggcggacgtttggatttaaacgggcattcgctttcgttccaccgtattcaaaataccgatgaaggggcgatgattgtcaaccacaatcaagacaaagaatccaccgttaccattacaggcaataaagatattgctacaaccggcaataacaacagcttggatagcaaaaaagaaattgcctacaacggttggtttggcgagaaagatacgaccaaaacgaacgggcggctcaaccttgtttaccagcccgccgcagaagaccgcaccctgctgctttccggcggaacaaatttaaacggcaacatcacgcaaacaaacggcaaactgtttttcagcggcagaccaacaccgcacgcctacaatcatttaaacgaccattggtcgcaaaaagagggcattcctcgcggggaaatcgtgtgggacaacgactggatcaaccgcacatttaaagcggaaaacttccaaattaaaggcggacaggcggtggtttcccgcaatgttgccaaagtgaaaggcgattggcatttgagcaatcacgcccaagcagtttttggtgtcgcaccgcatcaaagccacacaatctgtacacgttcggactggacgggtctgacaaattgtgtcgaaaaaaccattaccgacgataaagtgattgcttcattgactaagaccgacatcagcggcaatgtcgatcttgccgatcacgctcatttaaatctcacagggcttgccacactcaacggcaatcttagtgcaaatggcgatacacgttatacagtcagccacaacgccacccaaaacggcaaccttagcctcgtgggcaatgcccaagcaacatttaatcaagccacattaaacggcaacacatcggcttcgggcaatgcttcatttaatctaagcgaccacgccgtacaaaacggcagtctgacgctttccggcaacgctaaggcaaacgtaagccattccgcactcaacggtaatgtctccctagccgataaggcagtattccattttgaaagcagccgctttaccggacaaatcagcggcggcaaggatacggcattacacttaaaagacagcgaatggacgctgccgtcaggcacggaattaggcaatttaaaccttgacaacgccaccattacactcaattccgcctatcgccacgatgcggcaggggcgcaaaccggcagtgcgacagatgcgccgcgccgccgttcgcgccgttcgcgccgttccctattatccgttacaccgccaacttcggtagaatcccgtttcaacacgctgacggtaaacggcaaattgaacggtcagggaacattccgctttatgtcggaactcttcggctaccgcagcgacaaattgaagctggcggaaagttccgaaggcacttacaccttggcggtcaacaataccggcaacgaacctgcaagcctcgaacaattgacggtagtggaaggaaaagacaacaaaccgctgtccgaaaaccttaatttcaccctgcaaaacgaacacgtcgatgccggcgcgtggcgttaccaactcatccgcaaagacggcgagttccgcctgcataatccggtcaaagaacaagagctttccgacaaactcggcaaggcagaagccaaaaaacaggcggaaaaagacaacgcgcaaagccttgacgcgctgattgcggccgggcgcgatgccgtcgaaaagacagaaagcgttgccgaaccggcccggcaggcaggcggggaaaatgtcggcattatgcaggcggaggaagagaaaaaacgggtgcaggcggataaagacaccgccttggcgaaacagcgcgaagcggaaacccggccggctaccaccgccttcccccgcgcccgccgcgcccgccgggatttgccgcaactgcaaccccaaccgcagccccaaccgcagcgcgacctgatcagccgttatgccaatagcggtttgagtgaattttccgccacgctcaacagcgttttcgccgtacaggacgaattagaccgcgtatttgccgaagaccgccgcaacgccgtttggacaagcggcatccgggacaccaaacactaccgttcgcaagatttccgcgcctaccgccaacaaaccgacctgcgccaaatcggtatgcagaaaaacctcggcagcgggcgcgtcggcatcctgttttcgcacaaccggaccgaaaacaccttcgacgacggcatcggcaactcggcacggcttgcccacggcgccgttttcgggcaatacggcatcgacaggttctacatcggcatcagcgcgggcgcgggttttagcagcggcagcctttcagacggcatcggaggcaaaatccgccgccgcgtgctgcattacggcattcaggcacgataccgcgccggtttcggcggattcggcatcgaaccgcacatcggcgcaacgcgctatttcgtccaaaaagcggattaccgctacgaaaacgtcaatatcgccacccccggccttgcattcaaccgctaccgcgcgggcattaaggcagattattcattcaaaccggcgcaacacatttccatcacgccttatttgagcctgtcctataccgatgccgcttcgggcaaagtccgaacacgcgtcaataccgccgtattggctcaggatttcggcaaaacccgcagtgcggaatggggcgtaaacgccgaaatcaaaggtttcacgctgtccctccacgctgccgccgccaaaggcccgcaactggaagcgcaacacagcgcgggcatcaaattaggctaccgctggtaa

SEQ ID NO: 5 - protein, 1431 aa, Neisseria meningitidis
MRFTHTTPFCSVLSTLGLFAVSPAYSSIVRNDVDYQYFRDFAENKGAFTVGASNISIQDKQGKILGRVLNGIPMPDFRVSNRQTAIATLVHPQYVNSVKHNVGYGSIQFGNDTQNPEEQAYTYRLVSRNPHPDYDYHLPRLNKLVTEISPTALSSVPLLGNGQPKANAYLDTDRFPYFVRLGSGTQQVRKADGTRTRTAPAYQYLTGGTPLKVLGFQNHGLLVGGSLTDQPLNTYAIAGDSGSPLFAFDKHENRWVLAGVLSTYAGFDNFFNKYIVTQPEFIRSTIRQYETRLDVGLTTNELIWRDNGNGNSTLQGLNERITLPIANPSLAPQNDSRHMPSEDAGKTLILSSRFDNKTLMLADNINQGAGALQFDSNFTVVGKNHTWQGAGVIVADGKRVFWQVSNPKGDRLSKLGAGTLIANGQGINQGDISIGEGTVVLAQKAASDGSKQAFNQVGITSGRGTAVLADSQQIKPENLYFGFRGGRLDLNGNNLAFTHIRHADGGAQIVNHNPDQAATLTLTGNPVLSPEHVEWVQWGNRPQGNAAVYEYINPHRNRRTDYFILKPGGNPREFFPLNMKNSTSWQFIGNNRQQAAEQVAQAENARPDLITFGGYLGENAQTGKAAPSYSKTNEAAIEKTRHIANAAVYGRPEYRYNGALNLHYRPKRTDSTLLLNGGMNLNGEVLIEGGNMIVSGRPVPHAYDHQAKREPVLENEWTDGSFKAARFTLRNHARLTAGRNTAHLDGDITAYDLSGIDLGFTQGKTPECYRSYHSGSTHCTPNAVLKAENYRALPATQVRGDITLNDRSELRLGKAHLYGSIRAGKDTAVRMEADSNWTLSQSSHTGALTLDGAQITLNPDFANNTHNNRFNTLTVNGTLDGFGTFRFLTGIVRKQNAPPLKLEGDSRGAFQIHVKNTGQEPQTTESLALVSLNPKHSHQARFTLQNGYADLGAYRYILRKNNNGYSLYNPLKEAELQIEATRAEHERNQQAYNQLQATDISRQVQHDSDATRQALQAWQNSQTELARIDSQVQYLSAQLKQTDPLTGILTRAQNLCAAQGYSADICRQVAKAADTNDLTLFETELDTYIERVEMAESELDKARQGGDAQAVETARHAYLNALNRLSRQIHSLKTGVAGIRMPNLAELISRSANTAVSEQAAYNTGRQQAGRRIDRHLTDPQQQNIWLETGTQQTDYHSGTHRPYQQTTNYAHIGIQTGITDRLSVGTILTDERTNNRFDEGVSARNRSNGAHLFVKGENGALFAAADLGYSNSRTRFTDYDGAAVRRHAWDAGINTGIKIDTGINLRPYAGIRINRSNGNRYVLDGAEINSPAQIQTTWHAGIRLDKTVELGQAKLTPAFSSDYYHTRQNSGSALSVNDRTLLQQAAHGTLHTLQIDAGYKGWNAKLHAAYGKDSNTARHKQAGIKIGYNW

SEQ ID NO: 6 - DNA, 4296 bp, Neisseria meningitidis
  source 1..4296: /mol_type=“unassigned DNA” /organism=“Neisseria meningitidis”
atgcgcttcacacacaccaccccattttgttccgtattgtccaccctcggtctttttgccgtttcccctgcttactcatccattgtccgcaacgatgtcgattaccaatattttcgcgactttgccgaaaataaaggcgcgttcaccgtaggtgcaagcaatatttccatccaagacaagcaaggcaaaatattaggcagggttctcaacggcatccccatgcccgacttccgcgtcagcaaccgccaaaccgccatcgccaccctggttcacccccaatacgtcaacagtgtcaaacacaacgtcggctacggttccatacaattcggcaacgacacccaaaatccagaagaacaagcctatacctaccgcctcgtatcacgcaacccgcacccggactacgactaccaccttccccgcctcaacaaactggttaccgaaatctcacctaccgcactcagcagcgtacccttgcttggaaacggccagccaaaggccaatgcctacctcgataccgaccgcttcccctactttgtacgactcggctcaggcacgcaacaagtccgcaaagcagacggcacgcgtacacgaaccgccccggcataccaatacctgaccggcggcacgccgctgaaagtattggggttccaaaaccacggcttactcgtcggcggcagcctgaccgaccaaccccttaacacctacgcaatcgccggagacagcggttcccccctgtttgccttcgacaagcatgaaaaccgctgggtgcttgcgggcgtactcagcacctacgccggcttcgataatttcttcaacaaatacatcgtcacgcaacccgaattcatccgttccaccatccgccaatacgaaacccggctggatgtcgggctgaccaccaacgaactcatatggcgcgacaacggtaatggcaacagcaccctgcaagggctcaacgaacgcatcaccctgcccattgcaaacccttcgcttgccccacaaaacgacagcaggcacatgccgtctgaagatgccggcaaaacgctcatcctatccagcaggttcgacaacaaaacactgatgctggcagacaatatcaaccaaggcgcaggcgcattgcagttcgacagcaacttcaccgtcgtcggtaaaaaccacacatggcaaggtgcaggcgttatcgtagccgacggcaaacgcgtcttctggcaagtcagcaaccccaaaggcgaccggctctccaaactgggcgcaggcacgcttatcgccaacggacaaggcatcaaccagggcgacatcagcatcggggaaggcactgtcgtactcgcccaaaaagctgcttcagacggcagcaaacaagcattcaaccaagtcggcatcaccagcggcaggggcacggccgtcctcgccgacagccagcaaatcaaacccgaaaacctctatttcggcttcaggggcggacggctcgacctcaacggcaacaaccttgcctttacccatatccgccatgcggacggcggcgcgcaaatcgtcaatcacaaccctgaccaagccgcgacactgacgctgaccggcaaccccgtcctcagtcccgagcatgtcgagtgggtgcaatggggcaaccgtccgcaaggcaacgcggcggtttacgaatacatcaacccgcaccgcaaccgtcggaccgactacttcatactcaaacccggcggcaacccgcgcgaatttttcccgttaaatatgaaaaactcaacaagctggcaatttatcggcaacaacaggcaacaggccgccgaacaagtcgcccaagccgaaaatgcccgccccgacctgattaccttcggcggatacttgggtgaaaacgcgcaaacgggcaaagccgcgccgagttacagcaaaaccaatgaagcagccatagaaaaaacccgccatatcgcaaatgccgccgtatacggccggcccgaataccgttacaacggcgcactcaacctgcactatcgtcccaaacgcaccgacagcacgctgttgctcaacggcggcatgaaccttaacggggaagtcttgattgagggcggcaatatgattgtgtcaggcaggcccgtaccccatgcctacgaccaccaggccaaacgcgaacccgttcttgaaaacgaatggaccgacggcagcttcaaggctgcacggttcaccctgcgaaaccatgcccgactgacggcagggcgcaataccgcgcatctggacggcgacataaccgcatacgatctgtccggcatcgacctcggctttacccaaggcaaaacaccggaatgctaccgctcctaccatagcggcagcacccactgcacacccaacgccgttttaaaagccgaaaactatcgtgcactacctgcaacgcaagtacgcggcgacattacccttaacgaccgttcagagctccgcctgggcaaagcacacctgtacggcagcatccgtgccggcaaagacaccgcagtccgcatggaagcagacagcaactggacactttcccagtccagccacaccggcgcactgacgcttgacggcgcacaaattaccctgaaccccgatttcgccaataatacacacaacaaccgcttcaacacactgaccgtcaacggcacacttgacgggttcggcacattccgattcctgaccggcatcgtccgaaaacaaaatgccccccccctcaaactggaaggggacagccgcggcgcattccaaatccacgtcaaaaacaccggacaagaacctcaaacaaccgaatcgcttgcacttgtgagcctcaatccgaaacacagccaccaagcccgattcaccctccaaaacggctatgccgatttgggtgcctaccgctacatcctccgcaaaaacaacaacggatacagcctgtacaacccgctcaaagaggccgaacttcaaattgaagccacgcgtgcggaacatgagcgcaaccaacaggcatacaaccaattacaggcaaccgacatcagcagacaggttcaacatgactctgacgcgaccaggcaggcactacaggcctggcagaacagtcaaaccgaacttgcccgcatcgacagccaagtccaatatctgtccgcccaattgaaacagacagacccgctgaccggcattctgacgcgtgcccaaaacctgtgtgccgcacaaggatacagtgccgatatctgccgtcaggttgccaaagccgccgacacgaacgacctgacactcttcgaaaccgaactggatacgtatatagaacgtgtagaaatggccgaatccgaacttgacaaagcacggcaaggcggcgatgcgcaagccgtcgaaacagcccggcacgcctacctgaacgcactcaaccgtctgtcccgacaaatccacagtttgaaaaccggcgttgccggcatccgtatgccgaacctggccgaactgatcagccggtcggccaacaccgccgtttccgaacaggccgcctacaataccggccggcaacaggcgggacgccgcatcgaccgccaccttaccgatccgcagcagcaaaacatctggctggaaaccggtacgcaacaaaccgactaccatagcggcacacaccgtccctaccaacaaactaccaactatgcacatatcggcatccaaaccggcatcaccgaccgtctcagtgtcggtacgattttaaccgatgagcgcacaaacaaccgttttgatgaaggcgtatccgcccgaaaccgcagcaacggcgcacatctgttcgtcaaaggggaaaacggcgcactctttgccgcggcagatttaggctacagcaacagccgtacccgatttaccgattatgacggggctgccgtccgccgccacgcatgggatgcaggcatcaacaccggcatcaaaatcgataccggcatcaacctcagaccctatgccggcatccgtataaaccgcagcaacggcaaccggtacgtactcgacggcgcagagataaacagcccggcgcaaatccaaaccacatggcatgccggcatccgtctcgataaaaccgtcgaactgggtcaagccaagctgacccccgccttcagcagcgattactaccatacccgccaaaacagcggttccgccctcagcgtcaacgaccgtaccttactgcagcaagccgcccacggcacactgcataccctgcaaatcgacgccggatacaaaggctggaacgccaaacttcatgccgcttacggcaaagacagcaacaccgcccgccacaaacaggcaggaatcaaaataggctacaactggtaa